SEQUENCE LISTING (43 sequences)

SEQ ID NO: 1 - protein, 47 aa, Artificial Sequence
  Synthetic construct
PEETHKGRTVPAHVETVVLFFPDVWHCLPTRSEWETLSRGYKQQLVE

SEQ ID NO: 2 - protein, 41 aa, Artificial Sequence
  Synthetic construct
SEEKRKLAQLQVAYHQLFQEYDNHIKSSVVGSERKRGMQLE

SEQ ID NO: 3 - protein, 259 aa, Artificial Sequence
  Synthetic construct
NRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQL

SEQ ID NO: 4 - protein, 47 aa, Artificial Sequence; identical to SEQ ID NO: 1
  Synthetic construct

SEQ ID NO: 5 - protein, 51 aa, Artificial Sequence
  Synthetic construct
YHRPEETHKGRTVPAHVETVVLFFPDVWHCLPTRSEWETLSRGYKQQLVEK

SEQ ID NO: 6 - protein, 30 aa, Artificial Sequence
  Synthetic construct
HRPEETHKGRTVPAHVETVVLFFPDVWHCL

SEQ ID NO: 7 - protein, 40 aa, Artificial Sequence
  Synthetic construct
EEKRKLAQLQVAYHQLFQEYDNHIKSSVVGSERKRGMQLE

SEQ ID NO: 8 - protein, 35 aa, Artificial Sequence
  Synthetic construct
AEIRYHRPEETHKGRTVPAHVETVVLFFPDVWHCL

SEQ ID NO: 9 - protein, 80 aa, Artificial Sequence
  Synthetic construct
MHHHHHHKLYGRKKRRQRRRGSYPYDVPDYAGSPEETHKGRTVPAHVETVVLFFPDVWHCLPTRSEWETLSRGYKQQLVE

SEQ ID NO: 10 - protein, 103 aa, Artificial Sequence
  Synthetic construct
RPEETHKGRTVPAHVETVVLFFPDVWHCLPTRSEWETLSRGYKQQLVEKLQGERKEADGEQDEEEKDDGEAKEISTPTHWSKLDPKTMKVNDLRKELESRALS

SEQ ID NO: 11 - protein, 1150 aa, Artificial Sequence [sequence omitted]

SEQ ID NO: 12 - protein, 1146 aa, Artificial Sequence
  Synthetic construct
MAQFGGQKNPPWATQFTATAVSQPAALGVQQPSLLGASPTIYTQQTALAAAGLTTQTPANYQLTQTAALQQQAAAVLQQQYSQPQQALYSVQQQLQQPQQTILTQPAVALPTSLSLSTPQPAAQITVSYPTPRSSQQQTQPQKQRVFTGVVTKLHDTFGFVDEDVFFQLGAVKGKTPQVGDRVLVEATYNPNMPFKWNAQRIQTLPNQNQSQTQPLLKTPTAVIQPIVPQTTFGVQAQPQPQSLLQAQISAASITPLLQTQPQPLLQQPQQKAGLLQPPVRIVSQPQPARRLDPPSRFSGRNDRGDQVPNRKDDRSRERDRERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAVLDPPDADHLYSAKVMLMASPSMEDLYHKSCALAEDPQDLRDGFQHPARLVKFLVGMKGKDEAMAIGGHWSPSLDGPNPEKDPSVLIKTAIRCCKALTGIDLSVCTQWYRFAEIRYHRPEETHKGRTVPAHVETVVLFFPDVWHCLPTRSEWETLSRGYKQQLVEKLQGERKKADGEQDEEEKDDGEVKEIATPTHWSKLDPKAMKVNDLRKELESRALSSKGLKSQLIARLTKQLKIEEQKEEQKELEKSEKEEEDEDDKKSEDDKEEEERKRQEEVERQRQERRYILPDEPAIIVHPNWAAKSGKFDCSIMSLSVLLDYRLEDNKEHSFEVSLFAELFNEMLQRDFGVRIYKSLLSLPEKEDKKDKEKKSKKEERKDKKEEREDDIDEPKPKRRKSGDDKDKKEDRDERKKEEKRKDDSKDDDETEEDNNQDEYDPMEAEEAEDEDDDREEEEVKRDDKRDVSRYCKDRPAKDKEKEKPQMVTVNRDLLMAFVYFDQSHCGYLLEKDLEEILYTLGLHLSRAQVKKLLNKVVLRESCFYRKLTDTSKDDENHEESEALQEDMLGNRLLLPTPTIKQESKDGEENVGLIVYNGAMVDVGSLLQKLEKSEKVRAEVEQKLQLLEEKTDEDGKTILNLENSNKSLSGELREVKKDLGQLQENLEVSENMNLQFENQLNKTLRNLSTVMDDIHTVLKKDNVKSEDRDEKSKENGSGV

SEQ ID NO: 13 - protein, 1175 aa, Artificial Sequence
  Synthetic construct
MFFAAYQDVRRCYRRQTSEDFYPPFIMAQFGGQKNPPWATQFTATAVSQPAALGVQQPSLLGASPTIYTQQTALAAAGLTTQTPANYQLTQTAALQQQAAAAAAALQQLQQPQQTLLTQPAVALPTSLSLSTPQPAAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQAQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQALNANPFFYFRFSQDEEEKDDGEAKEISTPTHWSKLDPKTMKVNDLRKELESRALSSKGLKSQLIARLTKQLKVEEQKEEQKELEKSEKEEEEEDDRKSEDDKEEEERKRQEEMERQRRERRYILPDEPAIIVHPNWAAKSGKFDCSIMSLSVLLDYRLEDNKEHSFEVSLFAELFNEMLQRDFGVRIYKSLLSLPEKEDKKEKEKKSKKDERKDKKEDRDDETDEPKPKRRKSGDDKDKKEDRDERKKEDKRKEDSKDDDETEEDNNQDEYDPMEAEEAEDEEDDRDEEEINKRDDKRDINRYCKERPSKDKEKEKTQMITINRDLLMAFVYFDQSHCGYLLEKDLEEILYTLGLHLSRAQVKKLLNKVVLRESCFYRKLTDTSKDEENHEESEALQEDMLGNRLLLPTPTVKQESKDVEENVGLIVYNGAMVDVGSLLQKLEKSEKVRAEVEQKLQLLEEKTDEDEKTILNLENSNKSLSGELREVKKDFSQLQENLKISENMNLQFENQLNKTIRNLSTVMDEIHTVLKKDNVKNEDKDQKSKENGASV

SEQ ID NO: 14 - protein, 1508 aa, Artificial Sequence
  Synthetic construct
MWRRGAAWRKRGKLAHAPKADGFEMASMLAGTRLRPGAASPTPTARLFRCPQRPSASAWLRCSPPPHCSRAAAVLPSWPPGPGHRGCSRRRGSWGIGAFSVRGKRAQGSRDPSSVVGRWVPPSVAGGRHGAGTGGRWTAELWPLRVAAAEEGVRGRRIFAFSAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQALNANPFFYFRFSQAQEHSSSHGYLKLDNHKSERFEISGYVATSLDEEEKDDGEAKEISTPTHWSKLDPKTMKVNDLRKELESRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEKEDKRKDDSKDDDETEEDNNQDEYDPMEAEEAEDEEDDEDEKTILNLENSNKSLSGELREVKKDLSQLQENLKISENMNLQFENQLNKTIRNLSTVMDEIHTVLKKYLRPWGTDVEGYSSTSTNHQAPKLYVGSERPCNGPYCIASETSWSLVSISTGCSSWLLTWNGPKARSKASLPALGTPGAAVRTADGRSQALQEAAGSPRTWKSPRARPWGKGSSGPRGGWKSRASPGGRVGLGCGERSRTLGSGISSTALRRPKHGCPTPGPPGAVGPAPWSSVPPAASAADPRAVGPSSRRASGVVAAALAEALRCGLPAAGESMARPVQLAPGSLALVLCRLEAQKAAGAAEEPGGRAVFRAFRRANARCFWNARLARAASRLAFQGWLRRWVLLVRAPPACLQICSGRHSGFHVLQCGGLGSGPSSFGVVNFLGKTSDVFPVQMNPITQSQFVPLGEVLCCAISDMNTAQIVVTQESLLERLMKHYPGIAIPSEDILYTTLGTLIKERKIYHTGEGYFIVTPQTYFITNTTTQENKRMLPSDESRLMPASMTYLDTESGI

SEQ ID NO: 15 - protein, 1157 aa, Artificial Sequence
  Synthetic construct
MAQFGGQKNPPPWATQFTATAVSQPGPLAVQQSSLLGASPTIYTQQSALAAAGLASPSPANYQLSQTAALQQQAAAAAAAAAAALQQQYTQPQQTIYSVQQQLQPPPQAILTQPAVALPTSLALSTPQQAAQITVSYPTPRSNQQQTQPQKQRVFTGVVTKLHETFGFVDEDVFFQLTAVKGKSPQAGDRVLVEATYNPNMPFKWNAQRIQTLPNQNPASAQSLIKNPAAVMQPVAQPTAYAVQTQPPPQAQTLLQAQISAATLTPLLQTQTSPLLQQPQQKAGLLQTPVRIVSQPQPVRRIEPPSRFSVRNDRGDSILSRKDDRNRERERERRRSRDRSPQRKRSRERSPRRERERSPRRPRRVVPRYTVQISKFCLDCPGCDTMELRRRYQNLYIPSDFFDAQFTWVDAFPISRPFQLGNYSNFYIMHKEVDPLEKNTAIVDPPDADHTYSAKVMLLASPSLEELYHKSCALAEDPIEVREGFQHPARLIKFLVGMKGKDEAMAIGGHWSPSLDGPNPDKDPSVLIRTAVRCCKALTGIELSLCTQWYRFAEIRYHRPEETHKGRTVPAHVETVVLFFPDVWHCLPTRSEWENLCHGYKQQLVDKLQGDRKEADGEQEEEDKEDGDAKEISTPTHWSKLDPKIMKVNDLRKELESRTLSSKGLKSQLIARLTKQLRIEEQKEEQKELEKCEKEEEEEEERKSEDDKEEEERKRQEELERQRREKRYMLPDEPAIIVHPNWSAKNGKFDCSIMSLSVLLDYRIEDNKEHSFEVSLFAELFNEMLQRDFGVRIYRELLALPEKEEKKDKEKKCKKEDKRERKEDKDDDDEPKPKRRKSSDDKIKLEEKEERKRDDRRKEDYREEDDPDYENQDDYEPIAAEEDDGDYDDREDDDDDSSSKDKREDKRDGNRYSKERQSKDKEKDKKQMVTVNRDLLMAFVYFDQSHCGYLLEKDLEEILYTLGLHLSRAQVKKLFTKILLKESLLYRKLTDTATEDGSHEETDPLHNDILGNCSLLPSKAVRTGLSTVEDKGGLIVYKGAMVDVGSLLQKLEKSEKTRTELEHRLQTLESKTEEDEKTISQLEASNRNLSEELKQTKDDVGHLKDSLKAAEDTRSLYEDQLTNTIKNLSAAMGEIQVVLNKNPSTTEDQKSKENGSS

SEQ ID NO: 16 - protein, 1094 aa, Artificial Sequence
  Synthetic construct
MSNLSPFGGGKNPPWVRNAGQGIQNIQQQMLGQAMGSIGGQPMVQYQQQTQQVYQQSLGLQQPNITMASMATLGSNLPSGIAGQLYPQVATVSYPPPRALNTNAFQPSVAGVPQQVQQNVPSSSTKQRVFTGTVTQVYDNFGFVDEDVFFQTNACVKGSNPVVGDRVLVEASYNPSMPFKWSATRIQVLPMGNNNNNTNTQQNNQNTRQQQQQSQPQQNRTSGTYNAVPPPAENANNRFTTSATNANTASNRNKVGRVRERSPRERKNEEEEIERKRRREERIREREKKEERSPSRTRRSKSPRPRRRTRVVPRYMVQIPKIALDLPEADVLEIRRRYQNMYIPSDFFSTGFRWVDAFPPHMPFALNKPYVDPCSENTAVLEPSDADYLFSAKVMLISMPAMEEIYKRCCGVSEDRDPDRDYVHPTRLINFLVGLRGKNETMAIGGPWSPSLDGPNPEKDPSVLIRTAVRTCKALTGIDLSSCTQWYRFLELYYRRAETTHKSGRVVPSRVETVILFLPDVWSCVPIKLEWDGLQLSYKKQLERKLLRAASSPDDLDAANDTDEAAVADQKALPTSSHITFTFLLHYIIVQLFPITKLNFQYRLYLLIDPIADDPVPEKKDPTHYSELDPKSMNVTELRQELAARNLNCKGLKSQLLARLMKAITSEQAKEEGRQDDIEENDKDISPPPKEEEDKKFKDIKDHDEDRRKLCERERAALEKRYTLPESSHIIVHPSRMAKSGKFDCTVMSLSVLLDYRPEDTKDDDSIKDGRRDREKDGRKRKIKLYTHDPYLLLSFVYFDQTHCGYIFDKDIEELIYTLGLKLSRAQVRKLVQKVVTRDSLHYRKLTDRSKEDDLKDEKKDEKEIDKTDSIKIENEEEILRSLALGNKKLLPVFVGSGPPSKRVHREDAIIEQSDESIVSDGFVIYKGSLLDVEKLVSQLKRSEKARLDTEERLMELQHELCIVNEKSTKQTNNIKALSEDLKVYKDKLRNTDEKLKKVSSECHTYLTAVKNMYHIAAKMMQSDTKKVEVVEIQDEKVSEVNGSEIETKFKMDSRWGDNKVPIKKEFTETDKDKKCDNKVSIKKEIIETDKEKK

SEQ ID NO: 17 - protein, 51 aa, Artificial Sequence; identical to SEQ ID NO: 5
  Synthetic construct

SEQ ID NO: 18 - protein, 203 aa, Artificial Sequence
  Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEISTPTHWSKLDPKTMKVNDLRKELESRALS

SEQ ID NO: 19 - protein, 185 aa, Artificial Sequence
  Synthetic construct
SEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAP

SEQ ID NO: 20 - protein, 46 aa, Artificial Sequence
  Synthetic construct
IKTAIRCCKALTGIDLSVCTQWYRFAEIRYHRPEETHKGRTVPAHV

SEQ ID NO: 21 - protein, 50 aa, Artificial Sequence
  Synthetic construct
KLQGERKEADGEQDEEEKDDGEAKEISTPTHWSKLDPKTMKVNDLRKELE

SEQ ID NO: 22 - protein, 29 aa, Artificial Sequence
  Synthetic construct
RPEETHKGRTVPAHVETVVLFFPDVWHCL

SEQ ID NO: 23 - protein, 38 aa, Artificial Sequence
  Synthetic construct
SEEKRKLAQLQVAYHQLFQEYDNHIKSSVVGSERKRGM

SEQ ID NO: 24 - protein, 49 aa, Artificial Sequence
  Synthetic construct
HRPEETHKGRTVPAHVETVVLFFPDVWHCLPTRSEWETLSRGYKQQLVE

SEQ ID NO: 25 - protein, 4 aa, Artificial Sequence
  Synthetic construct
RKRG

SEQ ID NO: 26 - protein, 4 aa, Artificial Sequence
  Synthetic construct
RKRH

SEQ ID NO: 27 - protein, 198 aa, Artificial Sequence
  Synthetic construct
MAQFGGQKNPPWATQFTATAVSQPAALGVQQPSLLGASPTIYTQQTALAAAGLTTQTPANYQLTQTAALQQQAAAAAAALQQQYSQPQQALYSVQQQLQQPQQTLLTQPAVALPTSLSLSTPQPTAQITVSYPTPRSSQQQTQPQKQRVFTGVVTKLHDTFGFVDEDVFFQLSAVKGKTPQVGDRVLVEATYNPNMPF

SEQ ID NO: 28 - protein, 258 aa, Artificial Sequence
  Synthetic construct
PFKWNAQRIQTLPNQNQSQTQPLLKTPPAVLQPIAPQTTFGVQTQPQPQSLLQAQISAASITPLLQTQPQPLLQQPQQKAGLLQPPVRIVSQPQPARRLDPPSRFSGRNDRGDQVPNRKDDRSRERERERRRSRERSPQRKRSRERSPRRERERSPRRVRRVVPRYTVQFSKFSLDCPSCDMMELRRRYQNLYIPSDFFDAQFTWVDAFPLSRPFQLGNYCNFYVMHREVESLEKNMAILDPPDADHLYSAKVMLMAS

SEQ ID NO: 29 - protein, 296 aa, Artificial Sequence
  Synthetic construct
GERKEADGEQDEEEKDDGEAKEISTPTHWSKLDPKTMKVNDLRKELESRALSSKGLKSQLIARLTKQLKVEEQKEEQKELEKSEKEEDEDDDRKSEDDKEEEERKRQEEIERQRRERRYILPDEPAIIVHPNWAAKSGKFDCSIMSLSVLLDYRLEDNKEHSFEVSLFAELFNEMLQRDFGVRIYKSLLSLPEKEDKKEKDKKSKKDERKDKKEERDDETDEPKPKRRKSGDDKDKKEDRDERKKEDKRKGDSKDDDETEEDNNQDEYDPMEAEEAEDEEDDRDEEEMTKRDDKRD

SEQ ID NO: 30 - protein, 255 aa, Artificial Sequence
  Synthetic construct
KRDINRYCKERPSKDKEKEKTQMITINRDLLMAFVYFDQSHCGYLLEKDLEEILYTLGLHLSRAQVKKLLNKVVLRESCFYRKLTDTSKDEENHEESESLQEDMLGNRLLLPTPTVKQESKDVEENVGLIVYNGAMVDVGSLLQKLEKSEKVRAEVEQKLQLLEEKTDEDEKTILNLENSNKSLSGELREVKKDLSQLQENLKISENMSLQFENQMNKTIRNLSTVMDEIHTVLKKDNVKNEDKDQKSKENGASV

SEQ ID NO: 31 - protein, 174 aa, Artificial Sequence
  Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADGEQDEEEKDDGEAKEI

SEQ ID NO: 32 - protein, 159 aa, Artificial Sequence
  Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHRPEETHKGRTVPAHVETVVLFFPDVWHCLPTRSEWETLSRGYKQQLVEKLQGERKEADG

SEQ ID NO: 33 - protein, 101 aa, Artificial Sequence
  Synthetic construct
MASPSMEDLYHKSCALAEDPQELRDGFQHPARLVKFLVGMKGKDEAMAIGGHWSPSLDGPDPEKDPSVLIKTAIRCCKALTGIDLSVCTQWYRFAEIRYHR

SEQ ID NO: 34 - protein, 89 aa, Artificial Sequence
  Synthetic construct
RPEETHKGRTVPAHVETVVLFFPDVWHCLPTRSEWETLSRGYKQQLVEKLQGERKEADGEQDEEEKDDGEAKEISTPTHWSKLDPKTMK

SEQ ID NO: 35 - protein, 74 aa, Artificial Sequence
  Synthetic construct
RPEETHKGRTVPAHVETVVLFFPDVWHCLPTRSEWETLSRGYKQQLVEKLQGERKEADGEQDEEEKDDGEAKEI

SEQ ID NO: 36 - protein, 59 aa, Artificial Sequence
  Synthetic construct
RPEETHKGRTVPAHVETVVLFFPDVWHCLPTRSEWETLSRGYKQQLVEKLQGERKEADG

SEQ ID NO: 37 - protein, 30 aa, Artificial Sequence
  Synthetic construct
LFFPDVWHCLPTRSEWETLSRGYKQQLVEK

SEQ ID NO: 38 - protein, 30 aa, Artificial Sequence
  Synthetic construct
RGYKQQLVEKLQGERKEADGEQDEEEKDDG

SEQ ID NO: 39 - protein, 97 aa, Artificial Sequence
  Synthetic construct
SEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQ

SEQ ID NO: 40 - protein, 124 aa, Artificial Sequence
  Synthetic construct
ETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 41 - protein, 30 aa, Artificial Sequence
  Synthetic construct
EQDEEEKDDGEAKEISTPTHWSKLDPKTMK

SEQ ID NO: 42 - protein, 30 aa, Artificial Sequence
  Synthetic construct
WSKLDPKTMKVNDLRKELESRALSSKGLKS

SEQ ID NO: 43 - protein, 419 aa, Artificial Sequence
  Synthetic construct
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

(19) United States
(12) Patent Application Publication
(10) Pub. No.: US 2021/0106567 A1
(43) Pub. Date: Apr. 15, 2021

(54) COMPOSITIONS AND METHODS FOR INHIBITING CARP-1 BINDING TO NEMO

(71) Applicants: United States Government As Represented By The Department of Veterans Affairs, Washington, DC (US); Wayne State University, Detroit, MI (US)

(72) Inventors: ARUN K. RISHI, Troy, MI (US); Navnath Gavande, Detroit, MI (US)

(21) Appl. No.: 17/060,749

(22) Filed: Oct. 1, 2020

Related U.S. Application Data
(60) Provisional application No. 62/908,867, filed on Oct. 1, 2019.

(51) Int. Cl. A61K 31/427; A61K 47/54; A61K 31/40; A61K 31/4164; A61K 31/282

(52) U.S. Cl. CPC A61K 31/427; A61K 47/557; A61K 31/704; A61K 31/4164; A61K 31/282; A61K 31/40

(57) ABSTRACT

Described herein are compositions and methods for treating cancer in a subject. The compositions include selective NF-κB inhibitor inhibitors. The methods include inhibiting the binding of CARP-1 with NEMO.

### CROSS REFERENCE TO RELATED APPLICATIONS

This application claims the benefit of U.S. Provisional Application No. 62/908,867, filed Oct. 1, 2019. The content of this earlier filed application is hereby incorporated by reference herein in its entirety.

### STATEMENT REGARDING FEDERALLY FUNDED RESEARCH

This invention was made with government support under grant number 5I01BX001164-07 awarded by the Department of Veterans Affairs and under grant number 5P30CA022453-37 awarded by National Institutes of Health. The government has certain rights in the invention.

### INCORPORATION OF THE SEQUENCE LISTING

The present application contains a sequence listing that is submitted via EFS-Web concurrent with the filing of this application, containing the file name “37759_0257P1_SL.txt” which is 102,400 bytes in size, created on Sep. 15, 2020, and is herein incorporated by reference in its entirety.

### BACKGROUND

Diverse pathways of cell survival and apoptosis signaling by the transcription factor NF-κB are yet to be elucidated. CARP-1 (also referred to as CCAR1 or CCAR1/CARP1) is a perinuclear phospho-protein that regulates signaling by chemotherapy and growth factors. Doxorubicin, also known as Adriamycin, is a chemotherapeutic agent used to treat cancer. Doxorubicin works in part by interfering with the function of DNA. Resistance to doxorubicin among cancer cells is considered a barrier to effective treatment. Thus, effective cancer treatments are needed.

### SUMMARY

Disclosed herein are methods of treating of cancer, the methods comprising: administering to a subject with cancer a therapeutically effective amount of a CARP-1-NEMO inhibitor.

Disclosed herein are methods of inhibiting cell cycle progression, cell growth or DNA repair, the methods comprising: contacting a cancer cell or malignant tissue or administering to a subject with cancer a therapeutically effective amount of a CARP-1-NEMO inhibitor.

Disclosed herein are methods of enhancing a chemotherapeutic response in a subject, the methods comprising administering to a subject with cancer a therapeutically effective amount of a CARP-1-NEMO inhibitor.

Disclosed herein are methods of reducing chemotherapeutic toxicity in a subject, the methods comprising administering to a subject with cancer a therapeutically effective amount of a CARP-1-NEMO inhibitor and a therapeutically effective amount of chemotherapeutic agent.

Disclosed herein are methods of reducing or preventing chemotherapeutic resistance in a cancer cell, the methods comprising administering to a subject with cancer a therapeutically effective amount of a CARP-1-NEMO inhibitor and a therapeutically effective amount of chemotherapeutic agent.

Disclosed herein are methods of reducing systemic levels of one or more cytokines in a subject, the methods comprising administering to a subject with cancer a therapeutically effective amount of a CARP-1-NEMO inhibitor.

Disclosed herein are methods of inhibiting binding of NEMO to CARP-1, the methods comprising administering to a subject with cancer or contacting a cancer cell with a therapeutically effective amount of a CARP-1-NEMO inhibitor.

Disclosed herein are compositions for treating cancer, wherein the compositions comprise a CARP-1-NEMO inhibitor, a chemotherapeutic agent or a DNA damage-inducing agent, and optionally, a pharmaceutical carrier; wherein the CARP-1-NEMO inhibitor and the chemotherapeutic agent or a DNA damage-inducing agent are present in a therapeutically effective amount.

Disclosed herein are methods of enhancing the efficacy of radiotherapy and/or a chemotherapeutic agent, the methods comprising: administering to a subject with cancer: (a) an effective amount of radiotherapy and/or the chemotherapeutic agent; and (b) a therapeutically effective amount of a CARP-1-NEMO inhibitor, wherein the administration of the CARP-1-NEMO inhibitor enhances the efficacy of the chemotherapeutic agent and/or the radiotherapy in the subject with cancer.

Disclosed herein are methods for screening one or more compounds for pharmacological intervention in cancer, the methods comprising: (a) providing a CARP-1 amino acid fragment capable of binding to a NEMO amino acid fragment or a NEMO amino acid fragment capable of binding to a CARP-1 amino acid fragment; (b) providing a purified or non-purified compound; (c) screening the purified or non-purified compound in an environment that allows for binding of the compound or mixture of compounds in (b) to the CARP-1 amino acid fragment or to the NEMO amino acid fragment; and (d) isolating the compound in (c) that is bound to either the CARP-1 amino acid fragment or the NEMO amino acid fragment.

Disclosed herein are methods for screening one or more compounds for pharmacological intervention in cancer, the methods comprising: (a) providing a CARP-1 amino acid fragment capable of binding to a NEMO amino acid fragment or a NEMO amino acid fragment capable of binding to a CARP-1 amino acid fragment; (b) providing a purified or non-purified mixture of compounds; (c) screening the purified or non-purified mixture of compounds in an environment that allows for binding of a compound or mixture of compounds in (b) to the CARP-1 amino acid fragment or to the NEMO amino acid fragment; and (d) isolating the compound or mixture of compounds in (c) that is bound to either the CARP-1 amino acid fragment or the NEMO amino acid fragment.

Disclosed herein are compounds having a structure represented by a formula:

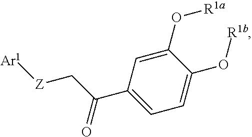

wherein Z is selected from —S(O)— and —SO₂—;

wherein each of R^{1a} and R^{1b} is independently selected from hydrogen and C1-C4 alkyl, or wherein each of R^{1a} and R^{1b} are covalently bonded, and, together with the intermediate atoms, comprise a 6-membered heterocycle;

or wherein each of R^{1a} and R^{1b} together comprise —CH₂—; and

wherein Ar¹ is a structure having a formula selected from:

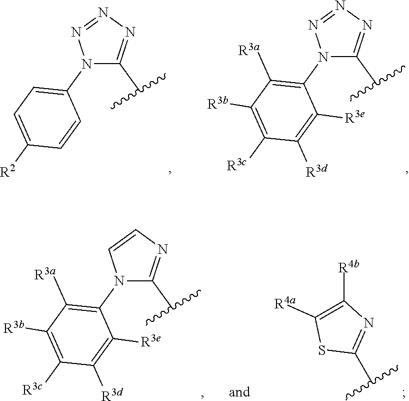

wherein R², when present, is C1-C4 alkyl;

wherein each of R^{3a}, R^{3b}, R^{3c}, R^{3d}, and R^{3e}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and

wherein each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar², provided that at least one of R^{4a} and R^{4b}, when present, is not hydrogen; and

wherein Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;

or wherein each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,

or a pharmaceutically acceptable salt thereof.

Disclosed herein are compounds having 2 a structure selected from:

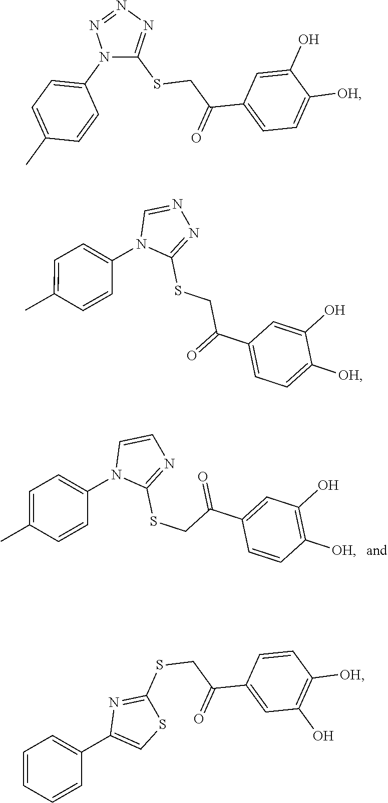

or a pharmaceutically acceptable salt thereof.

### BRIEF DESCRIPTION OF THE DRAWINGS

FIGS. 1A-D show CARP-1 binds with NEMO, and CARP-1 amino acids 553-599 (SEQ ID NO: 1) and NEMO amino acids 221-261 (SEQ ID NO: 2) harbor respective epitopes for interaction of CARP-1 and NEMO proteins. FIG. 1A shows protein complexes from the indicated cells were immunoprecipitated (I.P.) with noted antibodies followed by the analysis of the immunocomplexes by western blot (W.B.) using anti-NEMO (upper) antibodies. The membrane containing proteins from whole cell lysates was then probed with anti-CARP-1 (middle) or anti-NEMO (lower) antibodies for presence of respective proteins.

FIG. 1B shows W.B. analysis of I.P. protein complexes derived from using indicated antibodies from the noted cell lines. The membrane containing I.P proteins was probed with anti-CARP-1 antibodies (upper), while the membrane containing proteins from whole cell lysates was probed with anti-NEMO (middle) or anti-CARP-1 (lower) antibodies for presence of respective proteins. Arrowheads on the left or right side, respectively indicate presence of the proteins and molecular weight markers in panels A, B, of each blot. Schematic of CARP-1 WT and its various mutants (FIG. 1C) and NEMO WT and its mutants (FIG. 1D) that were utilized in co-I.P.-W.B. experiments to elucidate CARP-1 and NEMO interactions and map the respective minimal epitopes. The CARP-1 proteins have myc and 6×His epitopes at their carboxyl termini. The NEMO proteins, with the exception of 2-260 (SEQ ID NO: 3) and 221-261 mutants, harbored 6×myc epitope at their amino termini. NEMO 2-260 and 221-261 mutants had Gst epitope at their amino termini. Positive interactions are indicated by + and loss/absence of interaction is denoted by -.

FIGS. 2A-D show that the interference of CARP-1 binding with NEMO enhances chemotherapy efficacy in part by inhibiting activation of p65/RelA. FIGS. 2A, 2B and 2C show the indicated cell lines were treated with DMSO (Control), or with the noted dose and time of indicated agents. Determination of viable/live cells was carried out by MTT assays The bar chart columns represent means of two independent experiments; bars, SE. For panels A and C, *, @, p≤0.001 relative to respective vector sublines. FIG. 2D shows cells stably expressing myc-His-tagged wild-type CARP-1 or CARP-1 (Δ553-599; SEQ ID NO: 4) mutant were either treated with DMSO (control) or with various agents for indicated doses and time. Cell lysates were then analyzed by Western blot for levels of phosphorylated and total p65/RelA. Arrowheads on the left or right side indicate presence of proteins or molecular weight markers, respectively.

FIGS. 3A-B shows that interference of CARP-1 binding with NEMO inhibits activation of canonical NF-κB signaling. Indicated cells stably expressing wild-type or mutant CARP-1 protein were treated as in FIG. 2D for 1 h (FIG. 3A) or 6 h (FIG. 3B) durations. Cell lysates were then analyzed by W.B. for levels of CARP-1, phosphorylated and total p65/RelA, NEMO, IKKβ, and JNK1/2 proteins. The Western blot membranes in panels FIG. 3A and FIG. 3B were probed with anti-actin antibodies to assess protein loading. Arrowheads on the left or right side of each blot in panels FIGS. 3A and 3B indicate presence of proteins or molecular weight markers, respectively.

FIGS. 4A-E show the computational analyses of CARP-1 (551-600; SEQ ID NO: 5) binding with NEMO (221-261; SEQ ID NO: 2). FIG. 4A shows the SWISS Model image of CARP-1 (551-600; SEQ ID NO: 5). FIG. 4B shows the PDB ID: 3CL3 image of NEMO (221-261; SEQ ID NO: 2). FIG. 4C, FIG. 4D and FIG. 4E show the three top scoring docked complexes of CARP-1 (551-600; SEQ ID NO: 5) (Grey)/NEMO (221-261; SEQ ID NO: 2) (Green) in descending order, FIG. 4C, FIG. 4D, then FIG. 4E.

FIGS. 5A-F show the kinetics of CARP-1 binding with NEMO, and identification of pharmacologic inhibitors of CARP-1 interaction with NEMO. FIG. 5A depicts a SPR sensogram showing binding of CARP-1 (551-580; SEQ ID NO: 6) and NEMO (221-260; SEQ ID NO: 7) peptides. FIG. 5B shows the solution phase binding of Flag-CARP-1 (546-580; SEQ ID NO: 8) and Biotin-NEMO (221-261; SEQ ID NO: 2) peptides. The histogram shows fluorescence signal following binding of the two peptides over three noted times using AlphaLisa assay format. FIG. 5C and FIG. 5D show the structure and percent inhibition of binding of the Flag-CARP-1 (546-580; SEQ ID NO: 8) and Biotin-NEMO (221-261; SEQ ID NO: 2) peptides by respective compound, chemical name, formula, molecular weight, and abbreviated name of each compound that was identified following HTS. FIG. 5E shows that SNI-1 binds CARP-1. Left panel, His-TAT-HA-CARP-1 (551-580; SEQ ID NO: 6) was affinity-purified and immobilized on Ni-NTA beads, with or without SNI-1, washed 3× with RIPA buffer to remove free compound, and then allowed to bind with affinity-purified Gst-NEMO (221-261; SEQ ID NO: 2). Right panel, Gst-NEMO (221-261; SEQ ID NO: 2) peptide was affinity-purified and immobilized on glutathione sepharose, incubated with or without SNI-1, washed with RIPA buffer to remove free compound, and then allowed to bind with affinity-purified His-TAT-HA-CARP-1 (551-580; SEQ ID NO: 9). The complexes were analyzed by SDS-PAGE followed by WB with noted antibodies in respective top and middle blots. The lower blots in each panel indicate respective input peptides. FIG. 5F shows that SNI-1 does not affect NEMO interaction with RIPK1. HBC cells were untreated (control) or treated with indicated agents for noted dose and time. Protein complexes were immunoprecipitated (I.P.) with noted antibodies followed by the analysis of the immunocomplexes by western blot (W.B.) using anti-CARP-1 (upper blot), anti-NEMO (middle blot), and anti-RIPK1 (lower blot) antibodies. Arrowheads on the left or right side of each blot in the left panel indicate presence of proteins or molecular weight markers, respectively. The arrowheads on the left or right side of each blot in right panel indicate presence of molecular weight markers or proteins, respectively.

FIGS. 6A-F show that SNI-1 enhances anti-cancer efficacy of chemotherapy in vitro, and CARP-1 is important for cell growth suppression by SNI-1. FIG. 6A, FIG. 6B, FIG. 6C, FIG. 6D, FIG. 6E, and FIG. 6F show that cell viability was determined by MTT assay following treatments of cells with vehicle/DMSO (Control) or indicated time and doses of various agents. The columns in each histogram indicate percent of live/viable cells relative to their DMSO-treated controls and represent means of two-three independent experiments; bars, S.E. *, **, ***, p<0.001 relative to respective cells treated with chemotherapy alone (FIGS. 6A-D) or SNI-1 alone (FIG. 6E); * **, and ***, p<0.05, 0.01, and 0.001, respectively, relative to corresponding wild-type cells (FIG. 6F).

FIGS. 7A-G show that SNI-1 attenuates chemotherapy-induced phosphorylation/activation of RelA and NEMO, promotes RIPK1 cleavage, and enhances CARP-1 levels and apoptosis. FIG. 7A, FIG. 7B, FIG. 7C, FIG. 7D, FIG. 7E, FIG. 7F and FIG. 7G show that the indicated cells were either treated with DMSO (control), or treated with noted time and dose of respective agents. In FIG. 7F, cellular proteins were first separated into cyotosolic and nuclear fractions. The cell lysates were analyzed by Western blot for levels of phosphor-p65, p65, CARP-1, PARP, Cleaved Caspase-3, RIPK1, Actin, Lamin B, GAPDH, phospho-NEMO, and NEMO proteins.

FIGS. 8A-H show that SNI-1 attenuates chemotherapy-induced secretion of pro-inflammatory cytokines in vitro. FIG. 8A, FIG. 8B, FIG. 8C, FIG. 8D, FIG. 8E, FIG. 8F, FIG. 8G and FIG. 8H show that the indicated cells were either treated with DMSO (control), or treated with noted time and dose of respective agents. The media from the cells were analyzed by ELISA for levels of different pro-inflammatory cytokines.

FIGS. 9A-D show that SNI-1 enhances tumor suppression by Cisplatin in part by attenuating systemic levels of pro-inflammatory cytokines and promoting tumor apoptosis. Histogram columns showing median tumor volume (FIG. 9A) or percent T/C (percent treated (T)/control (C) tumor mass) (FIG. 9B) of the TNBC (4T1) xenograft-bearing mice treated with indicated agents. The xenograft establishment, treatment and analysis procedures were carried out as described herein. FIG. 9C shows the serum levels of noted pro-inflammatory cytokines. The columns in histograms indicate noted systemic cytokine levels in two representative animals from each of the control and treatment groups; bars, standard error (S.E.). FIG. 9D shows the immuno-histochemical staining of the noted proteins in the tumors derived from mouse with median tumor volume from each of the control and treatment groups in FIG. 9A.

FIG. 10 shows the schematic of the mechanism of action of SNI-1.

FIGS. 11A-D show the mapping of minimal epitopes of CARP-1 binding with NEMO. FIG. 11A shows that the noted cells were either untransfected (noted as −) or transfected separately with plasmids expressing indicated, myc-His-tagged CARP-1 mutant proteins in combination with plasmid expressing Gst-tagged NEMO (noted as +). Upper Blot, Western blot analysis of immunoprecipitation protein complexes using indicated antibodies. The membrane containing proteins from whole cell lysates was probed with anti-myc-Tag (middle) or anti-Gst (Lower) antibodies for presence of respective fusion proteins. FIG. 11B shows that the noted cells were either untransfected (noted as −) or transfected separately with plasmids expressing indicated, 6×myc-tagged NEMO mutant proteins in combination with plasmid expressing myc-His-tagged CARP-1 (552-654; SEQ ID NO: 10; noted as +). Upper Blot, Cell lysates were subjected to immunoprecipitation using anti-His tag antibodies to precipitate proteins complexed with His-tagged CARP-1 (552-654; SEQ ID NO: 10). The immunocomplexes were then analyzed by Western blot with anti-myc tag antibodies to detect 6×myc tagged NEMO proteins. The membrane containing proteins from whole cell lysates was probed with anti-His-Tag (Lower) antibodies for presence of CARP-1 (552-654; SEQ ID NO: 10) protein. FIG. 11C shows that the noted cells were first transfected with vector plasmid or plasmids encoding myc-His-tagged WT CARP-1 or its mutants as indicated. The neomycin-resistant, stable sublines were generated and characterized. Upper Blot, Western blot analysis of immunoprecipitation protein complexes using indicated antibodies. The membrane from upper blot was next probed with myc-Tag (middle) or the membrane containing proteins from whole cell lysates was probed with anti-NEMO (lower) antibodies.

FIG. 11D shows the Gst-tagged NEMO (2-260) protein, and various His-TAT-HA-tagged CARP-1 peptides were purified following expression in E. coli BL-21 cells. The Gst-NEMO (2-260; SEQ ID NO: 3) protein was immobilized on glutathione sepharose followed by incubation with IgG or indicated CARP-1 peptides. Following stringent washing, the bound proteins were analyzed by Western blot using anti-HA (upper) or anti-Gst (middle) antibodies. The lower blot shows respective HA-tagged CARP-1 peptides used as input. Arrowheads on the left or right side of each blot in panels FIGS. 11A-D indicate presence of proteins or molecular weight markers, respectively.

FIGS. 12A-I show that NEMO (221-261; SEQ ID NO: 2) interacts with CARP-1 (551-580; SEQ ID NO: 6). FIG. 12A, and FIGS. 12D-G show that the noted cells were either untransfected (noted wild-type) or transfected separately with indicated plasmids expressing eGFP, eGFP-tagged CARP-1 (551-580; SEQ ID NO: 6) mutant (FIG. 12A), Gst, Gst-NEMO (wild-type or mutant) proteins (FIGS. 12D-G), and neomycin-resistant, respective stable sublines were generated and characterized. Expression of respective, transfected proteins was analyzed by Western blot (upper blots), and each membrane was probed with anti-actin antibodies to assess protein loading (lower blots). FIGS. 12B-C show the Western blot analysis of immunoprecipitation protein complexes from indicated stable sublines using noted antibodies (upper, middle plots in FIG. 12B, and left blot in FIG. 12C). The membrane containing proteins from whole cell lysates was probed with anti-NEMO antibodies (lower blot in FIG. 12B) or anti-NEMO and anti-CARP-1 antibodies (right side blots in FIG. 12C). FIG. 12H shows that the indicated cells were either untransfected (noted as −) or transfected with Gst-NEMO (221-261; SEQ ID NO: 2) plasmid (noted as +), and Western blot analysis of immunoprecipitation protein complexes was conducted using noted antibodies (upper and lower right blots). The membrane containing proteins from whole cell lysates was probed with anti-CARP-1 (upper left) or anti-Gst (lower left) antibodies. Arrowheads on the left or right side of each blot in FIGS. 12A-G indicate presence of proteins or molecular weight markers, respectively. FIG. 12I shows the alignment of human CARP-1 (550-600; SEQ ID NO: 17) protein with corresponding regions of various vertebrate and invertebrate CARP-1 proteins deduced from GenBank sequences; human, SEQ ID NO: 11; mouse, SEQ ID NO: 12; dog, SEQ ID NO: 13; chimp, SEQ ID NO: 14; xenopus, SEQ ID NO: 15; and apis, SEQ ID NO: 16.

FIGS. 13A-C show that interference of CARP-1 binding with NEMO results in diminished RelA and NEMO phosphorylation. FIG. 13A shows HBC cells stably expressing Gst-NEMO or Gst-NEMO (221-261) were either untreated (Control) or treated with indicated dose and time of each agent, and Western blot analysis of the cell lysates was carried out using anti-phospho-RelA, anti-RelA, and anti-actin antibodies as in FIG. 3. FIG. 13B and FIG. 13C show that the indicated cells stably expressing wild-type or mutant CARP-1 protein were either untreated (control) or treated with noted dose and time of each agent. Cells were then processed for immunofluorescence staining for CARP-1 (red), DAPI (blue), and phosphorylated NEMO (green). Images were taken using Zeiss LSM 510 Meta NLO. Bar, 2 micrometer.

FIGS. 14A-C show the computational analyses of CARP-1 (551-600; SEQ ID NO: 5) binding with NEMO (221-261; SEQ ID NO: 2). Backbone RMSD and conformation histogram analyses for the three top scoring CARP1/NEMO complexes obtained from docking. FIG. 14A shows the top scoring pose. FIG. 14B shows the second pose. FIG. 14C shows the third pose. The calculations were done over the 24 ns production run.

FIGS. 15A-B show the buffer optimization (FIG. 15A) or DMSO tolerance (FIG. 15B) of the high-throughput screening assay. The Examples provide detail measurements of binding of CARP-1 (551-580; SEQ ID NO: 6) and Biotin-tagged NEMO (221-261; SEQ ID NO: 2), peptides in PBS buffer with or without Tween, BSG, or noted concentrations of DMSO.

FIGS. 16A-H show that 1-(3,4-dihydroxyphenyl)-2-{(1-(4-methylphenyl)-1H-tetrazol-5-yl)thio}ethanone (SNI-1) inhibits cell growth in time (FIG. 16A), dose-dependent matter (FIGS. 16B, C), and enhances efficacy of genotoxic chemotherapy in vitro in drug-resistant and BRCA-mutant TNBC, (FIGS. 16D-E), colon cancer cells (FIG. 16F), while NEMO is required for Adriamycin-dependent transcriptional activation of NF-κB (FIG. 16G) but not for γH2AX (FIG. 16H). FIG. 16A, and FIGS. D-F show that cell viability was determined by MTT assay following treatments of cells with vehicle/DMSO (Control) or indicated time and doses of various agents. The columns in each histogram indicate percent of live/viable cells relative to their DMSO-treated controls and represent means of two-three independent experiments. In FIGS. 16B, C, the columns in each histogram indicate number of live/viable cells; bars, S.E. ** and ***; p=0.005 and 0.001, respectively. FIG. 16G shows that for indicated cells were transfected with NF-κB-TATA-Luc plasmid followed by treatments with time and dose of noted agents. The columns in histogram indicate activities of the NF-κB reporter relative to the DMSO-treated controls and represent two separate experiments; bars, S.E. FIG. 16H shows that HeLa cells (wild-type, CARP-1 ko, and NEMO-ko) were either untreated (control) or treated with Adriamycin for indicated dose and time. Western blot analysis of the cell lysates was carried out using anti-γH2AX, anti-H2AX, and anti-actin antibodies. Arrowheads on the left or right side of each blot in FIG. 16H indicate presence of proteins or molecular weight markers, respectively.

FIGS. 17A-B show Adriamycin activates IKKα/β and caspase-3 independent of NEMO. HeLa cells (wild-type and NEMO-ko) were either untreated (control) or treated with indicated dose and time of noted agents. Western blot analysis of the cell lysates was carried out using anti-phospho RelA, anti-RelA, anti-cleaved caspase-3, anti-phospho-NEMO, anti-NEMO, anti-phospho-IKKα/β, anti-IKKα/β, and anti-actin antibodies. Arrowheads on the left or right side of each blot in FIGS. 17A-B indicate presence of proteins or molecular weight markers, respectively. Vertical bar in FIG. 17B denotes autoradiogram splicing.

FIGS. 18A-D show that 1-(3,4-dihydroxyphenyl)-2-{(1-(4-methylphenyl)-1H-tetrazol-5-yl)thio}ethanone (SNI-1) enhances tumor suppression by chemotherapy in part by attenuating systemic levels of pro-inflammatory cytokines and promoting tumor apoptosis. Histogram columns showing median tumor volume (FIG. 18A) or percent T/C (FIG. 18B) of the TNBC (4T1) xenograft-bearing mice treated with indicated agents. FIG. 18C and FIG. 18D show the serum levels of noted pro-inflammatory cytokines. The columns in histograms indicate noted systemic cytokine levels in two representative animals from each of the control and treatment groups; bars, S.E.

FIGS. 19A-B show that 1-(3,4-dihydroxyphenyl)-2-{(1-(4-methylphenyl)-1H-tetrazol-5-yl)thio}ethanone (SNI-1) administration does not induce apoptosis in various organs of 4T1 tumor-bearing mice. FIG. 19A is a histogram showing quantification of staining for indicated proteins in the tumor tissues in FIG. 9D. FIG. 19B shows immuno-histochemical staining for presence of cleaved caspase-3 was carried out in indicated murine tissues from a representative animal from control or SNI-1 group.

FIGS. 20A-B show that SNI-1 and its water soluble, di-sodium salt have similar activities, in vitro. FIG. 20A shows the chemical Structure of di-Sodium SNI-1. FIG. 20B shows cells that were treated with indicated dose and time of noted agent, and their viabilities determined by MTT assay. Columns represent means of three independent experiments; bars, SE. Please note that HCC1937 are representative of BRCA1-mutant TNBC.

FIG. 21 shows the chemical structures of SNI-1 analogs GL-208-213, GL215 and GL216. GL-214 is SNI-1.

FIGS. 22A-B show SNI-1 analogs enhance Adriamycin inhibition of TNBC cell growth. FIGS. 22A-B show cells treated with indicated dose of noted agent for 24 h, and their viabilities determined by MTT assay. Columns represent means of three independent experiments; bars, SE.

FIGS. 23A-B show that SNI-1 enhances Main 16C/Adr tumor suppression by Cisplatin. Histogram columns showing median tumor volume (FIG. 23A) and % T/C (FIG. 23B). The subcutaneous tumor bearing mice were treated with indicated agents. (Cisplatin, 3 mg/kg/dose, i.v., day 1, 5, 10, 14; SNI-1, 70 mg/kg/dose, i.p., daily days 1-13) The end-points for assessing anti-tumor activity involved qualitative determination via tumor growth inhibition (% T/C) where T is the median tumor volume of the treated mice and C is the median tumor volume of the Control mice on any given day of measurement. According to the NCI-accepted criteria, a treatment is considered effective if the T/C is <42%. It was found that % T/C remained consistently below 30 for the SNI-1+Cisplatin cohort on any day of the measurement indicated in (FIG. 23B).

FIG. 24 shows an efficacy study using human BRCA1 mutant SUM149 TNBC cell-derived xenografts in SCID mice.

FIGS. 25A-B show that SNI-1 enhances tumor suppression by Cisplatin Renal Cancer Syngeneic Tumor Model. Histogram columns showing median tumor volume (FIG. 25A) and % T/C (FIG. 25B). The subcutaneous tumor bearing mice were treated with indicated agents. (Cisplatin, 2 mg/kg/dose, i.v., day 3, 7, 11, 15; SNI-1, 70 mg/kg/dose, i.p., daily days 3-15) The dose of Cisplatin used was lower (subtherapeutic) than that (3 mg/kg/dose) used in other experiments. The end-points for assessing anti-tumor activity involved qualitative determination via tumor growth inhibition (% T/C) where T is the median tumor volume of the treated mice and C is the median tumor volume of the Control mice on any given day of measurement. According to the NCI-accepted criteria, a treatment is considered effective if the T/C is <42%. It was found that % T/C remained consistently below 40 for the SNI-1+Cisplatin cohort on any day of the measurement indicated in (FIG. 25B). Throughout this experiment, weight loss in animals ranged from 1.9-3.7% in Control group, 0.4-4.0% in SNI-1 group, 0.8-3.2% in Cisplatin group, and 1.6-6.7% in SNI-1+Cisplatin group. The animal weight loss remained below the NCI-accepted criteria od<10%.

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to an administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “CARP-1” is used interchangeably with “cell cycle and apoptosis regulatory protein 1”. The amino acid sequence of CARP-1 can be found in Table 1.

As used herein, the term “NEMO” is used interchangeably with “NF-kappa-B essential modulator”, “NF-κB essential modulator”, “NF-κB activating kinase IKK subunit γ” and “inhibitor of nuclear factor kappa-B kinase subunit gamma (IKK-γ)”. NEMO refers to a protein that in humans is encoded by the IKBKG gene. NEMO is a subunit of the IκB kinase complex that activates NF-κB. The human gene for IKBKG is located on chromosome Xq28. In vivo, NEMO activates NF-κB resulting in activation of genes involved in inflammation, immunity, cell survival, and other pathways. The amino acid sequence of NEMO can be found at Table 1. The Accession number for the nucleic acid sequence of NEMO is #NM_001099857.

As used herein, the term “CARP-1-NEMO inhibitor” is used interchangeably with “cell cycle and apoptosis regulatory protein (CARP)-1—NF-κB activating kinase IKK subunit γ (NEMO) inhibitor”, “cell cycle and apoptosis regulatory protein 1 (CARP-1)—NF-κB activating kinase IKK subunit γ (NEMO) inhibitor” and “cell cycle and apoptosis regulatory protein-1 (CARP-1)—NF-κB activating kinase IKK subunit γ (NEMO) inhibitor”.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be cancer.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

CARP-1 is a ubiquitous, ˜130 kDa peri-nuclear phospho-protein (Rishi, A. K., et al. (2003) J Biol Chem 278, 33422-33435) that has homologs in vertebrates, Apis millifera, and the worm Caenorhabditis elegans. Lst3, the C. elegans ortholog of human CARP-1, is an agonist of Notch signaling that also functions as an inhibitor of the EGFR-MAPK pathway (Yoo et al. (2004) Science 303, 663-666). This EGFR pathway antagonism by Lst3 corroborated prior findings of CARP-1 requirement for EGFR inhibitor-induced apoptosis (Rishi, A. K., et al. (2006) J Biol Chem 281, 13188-13198). Additionally, CARP-1 promoter methylation as well as signaling by protein kinase A (PKA) regulated CARP-1 expression and function, respectively (Rishi, A. K., et al. (2006) J Biol Chem 281, 13188-13198; Jiang, Y., et al. (2010) J Mol Signal 5, 7; and Zhang et al. (2007) Mol Cancer Ther 6, 1661-1672). CARP-1 is a phospho-protein, and although the EGF as well as the ATM kinase signaling target specific serine residues of CARP-1 (Beausoleil, S. A., et al. (2004) Proc Natl Acad Sci USA 101, 12130-12135; Blagoev, B., et al. (2003) Nat Biotechnol 21, 315-318; and Matsuoka et al. (2007) Science 316, 1160-1166), the precise role(s) and kinase(s) of CARP-1 serine phosphorylation remain unclear. CARP-1 binds with the LIM protein Zyxin and regulates apoptosis in response to UV-C irradiation (Hervy et al. (2010) Genes Cancer 1, 506-515), while it also interacts with Necdin to regulate myoblast survival (Francois, et al. (2012) PLoS One 7, e43335). Further, recent studies found CARP-1 as a co-activator of the cell cycle regulatory APC/C E3 ligase (Puliyappadamba et al. (2011) J Biol Chem 286, 38000-38017), the steroid-thyroid family of nuclear receptors (Kim et al., (2008) Mol Cell 31, 510-519), the GR signaling during adipogenesis, β-catenin in colon cancer metastasis, or neurogenin3-mediated pancreatic endocrine differentiation (Ou et al. (2009) J Biol Chem 284, 20629-20637; Ou et al. (2014) J Biol Chem 289, 17078-17086; and Lu et al. (2012) Biochem Biophys Res Commun 418, 307-312). Interestingly, CARP-1 also co-activated tumor suppressor p53 to transduce the DNA-damage-induced transcriptional increase of CDKI p21WAF1 in breast cancer cells (Kim et al., (2008)Mol Cell 31, 510-519).

Chemotherapeutics such as Adriamycin (ADR) induce double-strand breaks (DSBs) while phosphorylation of H2AX at serine139 (γ-H2AX) by ATM/ATR functions to repair DSBs (Pommier et al. (2010) Chem Biol 17, 421-433; Fornari et al. (1994) Mol Pharmacol 45, 649-656; and Podhorecka et al. (2010) J Nucleic Acids 2010). ADR also promotes apoptosis in part by inducing JNK-dependent γH2AX (Picco et al. (2013) Genes Cancer 4, 360-368; and Lu et al. (2006) Mol Cell 23, 121-132). It was shown that ADR induced CARP-1 and γH2AX, and depletion of CARP-1 abrogated γH2AX increase by ADR (Sekhar et al. (2019) Cancers (Basel) 11). CARP-1 binds with H2AX, and abrogation of CARP-1/H2AX binding blocked ADR-induced inhibition of triple negative breast cancer (TNBC) and HeLa cells (Sekhar et al. (2019) Cancers (Basel) 11).

NF-κB is a pro-inflammatory transcription factor that is a regulator of the immune system, and is responsive to a large number of stimuli that engage signaling pathways to activate this transcription factor and effect distinct cellular responses (Graef et al., (2001) Proc Natl Acad Sci USA 98, 5740-5745). With the exception of C. elegans, the NF-κB signaling components exist in most multicellular organisms (Zhang et al. (2017) Cell 168, 37-57). In mammalian cells, five members of the NF-κB family include RelA (p65), RelB, c-Rel, p50/p105 (NF-κB1), and p52/p100 (NF-κB2) that function by forming homo- and hetero-dimers. A family of inhibitory proteins called IκBs sequester the NF-κB complexes in the cytoplasm. IκBs are phosphorylated by IκB kinase (IKK), which leads to IκB degradation by ubiquitin-proteasome pathway, followed by release of NF-κB for its translocation to the nucleus where it functions as transcription factor (Zhang et al. (2017) Cell 168, 37-57). The IKK complex contains two kinase subunits, IKKα and IKKβ, and an associated regulatory subunit called NEMO (IKKγ). NF-κB regulates cellular homeostasis as well as tumor cell proliferation, survival, metastasis, inflammation, invasion, and angiogenesis, and often contributes to a resistant phenotype and poor prognosis (Liu et al. (2006) Mol Cell 21, 467-480). Although, a pro-apoptotic function for NF-κB has also been suggested (Shou et al. (2002) J Neurochem 81, 842-852; Martin et al. (2009) Aging (Albany N.Y.) 1, 335-349; and Ryan et al. (2000) Nature 404, 892-897), and possibly involves NF-κB-regulation of transducers of receptor-mediated apoptosis, a full characterization of the complex molecular details of the apoptotic functions of NF-κB remain to be accomplished. However, therapy-induced DNA damage that causes ATM/ATR activation to promote H2AX-dependent DSB repair, also stimulates phosphorylation of NEMO in the nucleus by ATM. The phosphorylated NEMO is mono-ubiquitinated, which triggers its nuclear export and IKK activation in the cytoplasm (Wu et al. (2006) Science 311, 1141-1146). This therapy-induced activation of canonical NF-κB promotes production of pro-inflammatory cytokines, cell growth and survival signaling, and contributes to therapy resistance.

Since, CARP-1 is a regulator of cell growth and survival signaling and a component of the NF-κB proteome, and CARP-1 depletion inhibited transcriptional activation of NF-κB by ADR, TNFα, or an experimental CARP-1 Functional Mimetic (CFM) compound, the molecular mechanism of CARP-1-dependent regulation of NF-κB signaling was investigated as described herein. It was determined that CARP-1 directly binds with NEMO, and blockage of this interaction interferes with ADR-induced activation of canonical NF-κB. Pharmacological inhibition of NEMO-CARP-1 binding enhances Cisplatin efficacy in part by impacting levels of circulating pro-inflammatory cytokines in immuno-competent mice bearing subcutaneous tumors of murine breast cancer cells.

Table 1 provides sequences of the various molecules described herein.

TABLE 1
Sequences.

| SEQ | | |
|---|---|---|
| ID NO: | NAME | SEQUENCE |
| 1 | CARP-1: | PEETHKGRTVPAHVETVVLFFPDVWHCLPTRSEWETL |
| | 553- | SRGYKQQLVE |
| | 599 | |
| 2 | NEMO: | SEEKRKLAQLQVAYHQLFQEYDNHIKSSVVGSERKRG |
| | 221-261 | MQLE |
| 3 | NEMO: | NRHLWKSQLCEMVQPSGGPAADQDVLGEESPLGKPA |
| | 2-260 | MLHLPSEQGAPETLQRCLEENQELRDAIRQSNQILRER |
| | | CEELLHFQASQREEKEFLMCKFQEARKLVERLGLEKL |
| | | DLKRQKEQALREVEHLKRCQQQMAEDKASVKAQVT |
| | | SLLGELQESQSRLEAATKECQALEGRARAASEQARQL |
| | | ESEREALQQQHSVQVDQLRMQGQSVEAALRMERQA |
| | | ASEEKRKLAQLQVAYHQLFQEYDNHIKSSVVGSERK |
| | | RGMQL |
| 4 | CARP-1: | PEETHKGRTVPAHVETVVLFFPDVWHCLPTRSEWETL |
| | 553- | SRGYKQQLVE |
| | 599 | |
| 5 | CARP-1: | YHRPEETHKGRTVPAHVETVVLFFPDVWHCLPTRSEW |
| | 550- | ETLSRGYKQQLVEK |
| | 600 | |
| 6 | CARP-1: | HRPEETHKGRTVPAHVETVVLFFPDVWHCL |
| | 551- | |
| | 580 | |
| 7 | NEMO: | EEKRKLAQLQVAYHQLFQEYDNHIKSSVVGSERKRGM |
| | 221-260 | QLE |
| 8 | CARP-1: | AEIRYHRPEETHKGRTVPAHVETVVLFFPDVWHCL |
| | 546- | |
| | 580 | |
| 9 | His- | MHHHHHHKLYGRKKRRQRRRGSYPYDVPDYAGSPEE |
| | Tat- | THKGRTVPAHVETVVLFFPDVWHCLPTRSEWETLSRG |
| | HA- | YKQQLVE |
| | CARP-1: | |
| | 553- | |
| | 599 | |
| 10 | CARP-1: | RPEETHKGRTVPAHVETVVLFFPDVWHCLPTRSEWETL |
| | 552- | SRGYKQQLVEKLQGERKEADGEQDEEEKDDGEAKEIS |
| | 654 | TPTHWSKLDPKTMKVNDLRKELESRALS |
| 11 | CARP-1: | MAQFGGQKNPPWATQFTATAVSQPAALGVQQPSLLGA |
| | human | SPTIYTQQTALAAAGLTTQTPANYQLTQTAALQQQAAA |
| | | AAAALQQQYSQPQQALYSVQQQLQQPQQTLLTQPAVAL |
| | | PTSLSLSTPQPTAQITVSYPTPRSSQQQTQPQKQRVFTGVV |
| | | TKLHDTFGFVDEDVFFQLSAVKGKTPQVGDRVLVEATY |
| | | NPNMPFKWNAQRIQTLPNQNQSQTQPLLKTPPAVLQPIA |
| | | PQTTFGVQTQPQPQSLLQAQISAASITPLLQTQPQPLLQQ |
| | | PQQKAGLLQPPVRIVSQPQPARRLDPPSRFSGRNDRGDQ |
| | | VPNRKDDRSRERERERRRSRERSPQRKRSRERSPRRERE |
| | | RSPRRVRRVVPRYTVQFSKFSLDCPSCDMMELRRRYQN |
| | | LYIPSDFFDAQFTWVDAFPLSRPFQLGNYCNFYVMHRE |
| | | VESLEKNMAILDPPDADHLYSAKVMLMASPSMEDLYH |
| | | KSCALAEDPQELRDGFQHPARLVKFLVGMKGKDEAMA |
| | | IGGHWSPSLDGPDPEKDPSVLIKTAIRCCKALTGIDLSVC |
| | | TQWYRFAEIRYHRPEETHKGRTVPAHVETVVLFFPDVW |
| | | HCLPTRSEWETLSRGYKQQLVEKLQGERKEADGEQDEE |
| | | EKDDGEAKEISTPTHWSKLDPKTMKVNDLRKELESRALS |
| | | SKGLKSQLIARLTKQLKVEEQKEEQKELEKSEKEEDEDDD |
| | | RKSEDDKEEEERKRQEEIERQRRERRYILPDEPAIIVHPNW |
| | | AAKSGKFDCSIMSLSVLLDYRLEDNKEHSFEVSLFAELFNE |
| | | MLQRDFGVRIYKSLLSLPEKEDKKEKDKKSKKDERKDKKE |
| | | ERDDETDEPKPKRRKSGDDKDKKEDRDERKKEDKRKGDSK |
| | | DDDETEEDNNQDEYDPMEAEEAEDEEDDRDEEEMTKRDDK |
| | | RDINRYCKERPSKDKEKEKTQMITINRDLLMAFVYFDQSHC |
| | | GYLLEKDLEEILYTLGLHLSRAQVKKLLNKVVLRESCFYRK |
| | | LTDTSKDEENHEESESLQEDMLGNRLLLPTPTVKQESKDVE |
| | | ENVGLIVYNGAMVDVGSLLQKLEKSEKVRAEVEQKLQLLE |
| | | EKTDEDEKTILNLENSNKSLSGELREVKKDLSQLQENLKISE |
| | | NMSLQFENQMNKTIRNLSTVMDEIHTVLKKDNVKNEDKDQK |
| | | SKENGASV |
| 12 | CARP-1: | MAQFGGQKNPPWATQFTATAVSQPAALGVQQPSLLGASPTIY |
| | mouse | TQQTALAAAGLTTQTPANYQLTQTAALQQQAAAVLQQQYSQ |
| | | PQQALYSVQQQLQQPQQTILTQPAVALPTSLSLSTPQPAAQITV |
| | | SYPTPRSSQQQTQPQKQRVFTGVVTKLHDTFGFVDEDVFFQLG |
| | | AVKGKTPQVGDRVLVEATYNPNMPFKWNAQRIQTLPNQNQSQ |
| | | TQPLLKTPTAVIQPIVPQTTFGVQAQPQPQSLLQAQISAASITPLL |
| | | QTQPQPLLQQPQQKAGLLQPPVRIVSQPQPARRLDPPSRFSGRN |
| | | DRGDQVPNRKDDRSRERDRERRRSRERSPQRKRSRERSPRRER |
| | | ERSPRRVRRVVPRYTVQFSKFSLDCPSCDMMELRRRYQNLYIP |
| | | SDFFDAQFTWVDAFPLSRPFQLGNYCNFYVMHREVESLEKNM |
| | | AVLDPPDADHLYSAKVMLMASPSMEDLYHKSCALAEDPQDLR |
| | | DGFQHPARLVKFLVGMKGKDEAMAIGGHWSPSLDGPNPEKDP |
| | | SVLIKTAIRCCKALTGIDLSVCTQWYRFAEIRYHRPEETHKGRT |
| | | VPAHVETVVLFFPDVWHCLPTRSEWETLSRGYKQQLVEKLQG |
| | | ERKKADGEQDEEEKDDGEVKEIATPTHWSKLDPKAMKVNDLR |
| | | KELESRALSSKGLKSQLIARLTKQLKIEEQKEEQKELEKSEKEEE |
| | | DEDDKKSEDDKEEEERKRQEEVERQRQERRYILPDEPAIIVHPN |
| | | WAAKSGKFDCSIMSLSVLLDYRLEDNKEHSFEVSLFAELFNEM |
| | | LQRDFGVRIYKSLLSLPEKEDKKDKEKKSKKEERKDKKEERED |
| | | DIDEPKPKRRKSGDDKDKKEDRDERKKEEKRKDDSKDDDETE |
| | | EDNNQDEYDPMEAEEAEDEDDDREEEEVKRDDKRDVSRYCK |
| | | DRPAKDKEKEKPQMVTVNRDLLMAFVYFDQSHCGYLLEKDL |
| | | EEILYTLGLHLSRAQVKKLLNKVVLRESCFYRKLTDTSKDDEN |
| | | HEESEALQEDMLGNRLLLPTPTIKQESKDGEENVGLIVYNGAM |
| | | VDVGSLLQKLEKSEKVRAEVEQKLQLLEEKTDEDGKTILNLEN |
| | | SNKSLSGELREVKKDLGQLQENLEVSENMNLQFENQLNKTLRN |
| | | LSTVMDDIHTVLKKDNVKSEDRDEKSKENGSGV |
| 13 | CARP-1: | MFFAAYQDVRRCYRRQTSEDFYPPFIMAQFGGQKNPPWATQFT |
| | dog | ATAVSQPAALGVQQPSLLGASPTIYTQQTALAAAGLTTQTPANY |
| | | QLTQTAALQQQAAAAAAALQQLQQPQQTLLTQPAVALPTSLSL |
| | | STPQPAAQITVSYPTPRSSQQQTQPQKQRVFTGVVTKLHDTFGF |
| | | VDEDVFFQLSAVKGKTPQVGDRVLVEATYNPNMPFKWNAQRI |
| | | QTLPNQNQSQTQPLLKTPPAVLQPIAPQTTFGVQAQPQPQSLLQ |
| | | AQISAASITPLLQTQPQPLLQQPQQKAGLLQPPVRIVSQPQPARR |
| | | LDPPSRFSGRNDRGDQVPNRKDDRSRERERERRRSRERSPQRKR |
| | | SRERSPRRERERSPRRVRRVVPRYTVQFSKFSLDCPSCDMMELR |
| | | RRYQNLYIPSDFFDAQFTWVDAFPLSRPFQLGNYCNFYVMHRE |
| | | VESLEKNMAILDPPDADHLYSAKVMLMASPSMEDLYHKSCAL |
| | | AEDPQELRDGFQHPARLVKFLVGMKGKDEAMAIGGHWSPSLD |
| | | GPDPEKDPSVLIKTAIRCCKALTGIDLSVCTQWYRFAEIRYHRPE |
| | | ETHKGRTVPAHVETVVLFFPDVWHCLPTRSEWETLSRGYKQQL |
| | | VEKLQGERKEADGEQALNANPFFYFRFSQDEEEKDDGEAKEIST |
| | | PTHWSKLDPKTMKVNDLRKELESRALSSKGLKSQLIARLTKQLK |
| | | VEEQKEEQKELEKSEKEEEEEDDRKSEDDKEEEERKRQEEMERQ |
| | | RRERRYILPDEPAIIVHPNWAAKSGKFDCSIMSLSVLLDYRLEDN |
| | | KEHSFEVSLFAELFNEMLQRDFGVRIYKSLLSLPEKEDKKEKEKK |
| | | SKKDERKDKKEDRDDETDEPKPKRRKSGDDKDKKEDRDERKKE |
| | | DKRKEDSKDDDETEEDNNQDEYDPMEAEEAEDEEDDRDEEEINK |
| | | RDDKRDINRYCKERPSKDKEKEKTQMITINRDLLMAFVYFDQSHC |
| | | GYLLEKDLEEILYTLGLHLSRAQVKKLLNKVVLRESCFYRKLTDT |
| | | SKDEENHEESEALQEDMLGNRLLLPTPTVKQESKDVEENVGLIVY |
| | | NGAMVDVGSLLQKLEKSEKVRAEVEQKLQLLEEKTDEDEKTILNL |
| | | ENSNKSLSGELREVKKDFSQLQENLKISENMNLQFENQLNKTIRNL |
| | | STVMDEIHTVLKKDNVKNEDKDQKSKENGASV |
| 14 | CARP-1: | MWRRGAAWRKRGKLAHAPKADGFEMASMLAGTRLRPGAASPTP |
| | chimp | TARLFRCPQRPSASAWLRCSPPPHCSRAAAVLPSWPPGPGHRGCSR |
| | | RRGSWGIGAFSVRGKRAQGSRDPSSVVGRWVPPSVAGGRHGAGTG |
| | | GRWTAELWPLRVAAAEEGVRGRRIFAFSAALGVQQPSLLGASPTIY |
| | | TQQTALAAAGLTTQTPANYQLTQTAALQQQAAAAAAALQQQYSQ |
| | | PQQALYSVQQQLQQPQQTLLTQPAVALPTSLSLSTPQPTAQITVSYP |
| | | TPRSSQQQTQPQKQRVFTGVVTKLHDTFGFVDEDVFFQLSAVKGK |
| | | TPQVGDRVLVEATYNPNMPFKWNAQRIQTLPNQNQSQTQPLLKTP |
| | | PAVLQPIAPQTTFGVQTQPQPQSLLQAQISAASITPLLQTQPQPLLQQ |
| | | PQQKAGLLQPPVRIVSQPQPARRLDPPSRFSGRNDRGDQVPNRKDD |
| | | RSRERERERRRSRERSPQRKRSRERSPRRERERSPRRVRRVVPRYTV |
| | | QFSKFSLDCPSCDMMELRRRYQNLYIPSDFFDAQFTWVDAFPLSRPF |
| | | QLGNYCNFYVMHREVESLEKNMAILDPPDADHLYSAKVMLMASPS |
| | | MEDLYHKSCALAEDPQELRDGFQHPARLVKFLVGMKGKDEAMAIG |
| | | GHWSPSLDGPDPEKDPSVLIKTAIRCCKALTGIDLSVCTQWYRFAEIR |
| | | YHRPEETHKGRTVPAHVETVVLFFPDVWHCLPTRSEWETLSRGYKQ |
| | | QLVEKLQGERKEADGEQALNANPFFYFRFSQAQEHSSSHGYLKLDN |
| | | HKSERFEISGYVATSLDEEEKDDGEAKEISTPTHWSKLDPKTMKVND |
| | | LRKELESRALSSKGLKSQLIARLTKQLKVEEQKEEQKELEKSEKEEDE |
| | | DDDRKSEDDKEEEERKRQEEIERQRRERRYILPDEPAIIVHPNWAAKS |
| | | GKFDCSIMSLSVLLDYRLEDNKEHSFEKEDKRKDDSKDDDETEEDNN |
| | | QDEYDPMEAEEAEDEEDDEDEKTILNLENSNKSLSGELREVKKDLSQL |
| | | QENLKISENMNLQFENQLNKTIRNLSTVMDEIHTVLKKYLRPWGTDVE |
| | | GYSSTSTNHQAPKLYVGSERPCNGPYCIASETSWSLVSISTGCSSWLLT |
| | | WNGPKARSKASLPALGTPGAAVRTADGRSQALQEAAGSPRTWKSPRA |
| | | RPWGKGSSGPRGGWKSRASPGGRVGLGCGERSRTLGSGISSTALRRPK |
| | | HGCPTPGPPGAVGPAPWSSVPPAASAADPRAVGPSSRRASGVVAAALA |
| | | EALRCGLPAAGESMARPVQLAPGSLALVLCRLEAQKAAGAAEEPGGRA |
| | | VFRAFRRANARCFWNARLARAASRLAFQGWLRRWVLLVRAPPACLQIC |
| | | SGRHSGFHVLQCGGLGSGPSSFGVVNFLGKTSDVFPVQMNPITQSQFVPL |
| | | GEVLCCAISDMNTAQIVVTQESLLERLMKHYPGIAIPSEDILYTTLGTLIK |
| | | ERKIYHTGEGYFIVTPQTYFITNTTTQENKRMLPSDESRLMPASMTYLDT |
| | | ESGI |
| 15 | CARP-1: | MAQFGGQKNPPPWATQFTATAVSQPGPLAVQQSSLLGASPTIYTQQS |
| | xenopus | ALAAAGLASPSPANYQLSQTAALQQQAAAAAAAAAAALQQQYTQP |
| | | QQTIYSVQQQLQPPPQAILTQPAVALPTSLALSTPQQAAQITVSYPTPR |
| | | SNQQQTQPQKQRVFTGVVTKLHETFGFVDEDVFFQLTAVKGKSPQA |
| | | GDRVLVEATYNPNMPFKWNAQRIQTLPNQNPASAQSLIKNPAAVMQ |
| | | PVAQPTAYAVQTQPPPQAQTLLQAQISAATLTPLLQTQTSPLLQQPQQ |
| | | KAGLLQTPVRIVSQPQPVRRIEPPSRFSVRNDRGDSILSRKDDRNRERE |
| | | RERRRSRDRSPQRKRSRERSPRRERERSPRRPRRVVPRYTVQISKFCLD |
| | | CPGCDTMELRRRYQNLYIPSDFFDAQFTWVDAFPISRPFQLGNYSNFY |
| | | IMHKEVDPLEKNTAIVDPPDADHTYSAKVMLLASPSLEELYHKSCAL |
| | | AEDPIEVREGFQHPARLIKFLVGMKGKDEAMAIGGHWSPSLDGPNPD |
| | | KDPSVLIRTAVRCCKALTGIELSLCTQWYRFAEIRYHRPEETHKGRTV |
| | | PAHVETVVLFFPDVWHCLPTRSEWENLCHGYKQQLVDKLQGDRKE |
| | | ADGEQEEEDKEDGDAKEISTPTHWSKLDPKIMKVNDLRKELESRTLS |
| | | SKGLKSQLIARLTKQLRIEEQKEEQKELEKCEKEEEEEEERKSEDDKE |
| | | EEERKRQEELERQRREKRYMLPDEPAIIVHPNWSAKNGKFDCSIMSL |
| | | SVLLDYRIEDNKEHSFEVSLFAELFNEMLQRDFGVRIYRELLALPEKE |
| | | EKKDKEKKCKKEDKRERKEDKDDDDEPKPKRRKSSDDKIKLEEKEE |
| | | RKRDDRRKEDYREEDDPDYENQDDYEPIAAEEDDGDYDDREDDDD |
| | | DSSSKDKREDKRDGNRYSKERQSKDKEKDKKQMVTVNRDLLMAFV |
| | | YFDQSHCGYLLEKDLEEILYTLGLHLSRAQVKKLFTKILLKESLLYRK |
| | | LTDTATEDGSHEETDPLHNDILGNCSLLPSKAVRTGLSTVEDKGGLIV |
| | | YKGAMVDVGSLLQKLEKSEKTRTELEHRLQTLESKTEEDEKTISQLE |
| | | ASNRNLSEELKQTKDDVGHLKDSLKAAEDTRSLYEDQLTNTIKNLSA |
| | | AMGEIQVVLNKNPSTTEDQKSKENGSS |
| 16 | CARP-1: | MSNLSPFGGGKNPPWVRNAGQGIQNIQQQMLGQAMGSIGGQPMVQ |
| | apis | YQQQTQQVYQQSLGLQQPNITMASMATLGSNLPSGIAGQLYPQVAT |
| | | VSYPPPRALNTNAFQPSVAGVPQQVQQNVPSSSTKQRVFTGTVTQV |
| | | YDNFGFVDEDVFFQTNACVKGSNPVVGDRVLVEASYNPSMPFKWS |
| | | ATRIQVLPMGTNTQQNNQNTRQQQQQSQPQQNRTSGTYNA |
| | | VPPPAENANNRFTTSATNANTASNRNKVGRVRERSPRERKNEEEEIE |
| | | RKRRREERIREREKKEERSPSRTRRSKSPRPRRRTRVVPRYMVQIPKI |
| | | ALDLPEADVLEIRRRYQNMYIPSDFFSTGFRWVDAFPPHMPFALNKP |
| | | YVDPCSENTAVLEPSDADYLFSAKVMLISMPAMEEIYKRCCGVSEDR |
| | | DPDRDYVHPTRLINFLVGLRGKNETMAIGGPWSPSLDGPNPEKDPSV |
| | | LIRTAVRTCKALTGIDLSSCTQWYRFLELYYRRAETTHKSGRVVPSR |
| | | VETVILFLPDVWSCVPIKLEWDGLQLSYKKQLERKLLRAASSPDDLD |
| | | AANDTDEAAVADQKALPTSSHITFTFLLHYIIVQLFPITKLNFQYRLY |
| | | LLIDPIADDPVPEKKDPTHYSELDPKSMNVTELRQELAARNLNCKGL |
| | | KSQLLARLMKAITSEQAKEEGRQDDIEENDKDISPPPKEEEDKKFKD |
| | | IKDHDEDRRKLCERERAALEKRYTLPESSHIIVHPSRMAKSGKFDCT |
| | | VMSLSVLLDYRPEDTKDDDSIKDGRRDREKDGRKRKIKLYTHDPYL |
| | | LLSFVYFDQTHCGYIFDKDIEELIYTLGLKLSRAQVRKLVQKVVTRD |
| | | SLHYRKLTDRSKEDDLKDEKKDEKEIDKTDSIKIENEEEILRSLALGN |
| | | KKLLPVFVGSGPPSKRVHREDAIIEQSDESIVSDGFVIYKGSLLDVEKL |
| | | VSQLKRSEKARLDTEERLMELQHELCIVNEKSTKQTNNIKALSEDLK |
| | | VYKDKLRNTDEKLKKVSSECHTYLTAVKNMYHIAAKMMQSDTKK |
| | | VEVVEIQDEKVSEVNGSEIETKFKMDSRWGDNKVPIKKEFTETDKDK |
| | | KCDNKVSIKKEIIETDKEKK |
| 17 | CARP-1: | YHRPEETHKGRTVPAHVETVVLFFPDVWHCLPTRSEWETLSRGYKQ |
| | 550- | QLVEK |
| | 600 | |
| 18 | CARP-1: | MASPSMEDLYHKSCALAEDPQELRDGFQHPARLVKFLVGMKGKDE |
| | 452- | AMAIGGHWSPSLDGPDPEKDPSVLIKTAIRCCKALTGIDLSVCTQWY |
| | 654 | RFAEIRYHRPEETHKGRTVPAHVETVVLFFPDVWHCLPTRSEWETLS |
| | | RGYKQQLVEKLQGERKEADGEQDEEEKDDGEAKEISTPTHWSKLDP |
| | | KTMKVNDLRKELESRALS |
| 19 | NEMO: | SEEKRKLAQLQVAYHQLFQEYDNHIKSSVVGSERKRGMQLEDLKQQ |
| | 221-405 | LQQAEEALVAKQEVIDKLKEEAEQHKIVMETVPVLKAQADIYKADF |
| | | QAERQAREKLAEKKELLQEQLEQLQREYSKLKASCQESARIEDMRK |
| | | RHVEVSQAPLPPAPAYLSSPLALPSQRRSPPEEPPDFCCPKCQYQAP |
| 20 | CARP-1: | IKTAIRCCKALTGIDLSVCTQWYRFAEIRYHRPEETHKGRTVPAHV |
| | 521- | |
| | 566 | |
| 21 | CARP-1: | KLQGERKEADGEQDEEEKDDGEAKEISTPTHWSKLDPKTMKVND |
| | 600- | LRKELE |
| | 650 | |
| 22 | CARP-1: | RPEETHKGRTVPAHVETVVLFFPDVWHCL |
| | 552- | |
| | 580 | |
| 23 | NEMO: | SEEKRKLAQLQVAYHQLFQEYDNHIKSSVVGSERKRGM |
| | 221-258 | |
| 24 | CARP-1: | HRPEETHKGRTVPAHVETVVLFFPDVWHCLPTRSEWETLSRGYK |
| | 551- | QQLVE |
| | 599 | |
| 25 | NEMO: | RKRG |
| | 254-257 | |
| 26 | NEMO: | RKRH |
| | 357-360 | |
| 27 | CARP-1: | MAQFGGQKNPPWATQFTATAVSQPAALGVQQPSLLGASPTIYTQQ |
| | 1- | TALAAAGLTTQTPANYQLTQTAALQQQAAAAAAALQQQYSQPQQ |
| | 198 | ALYSVQQQLQQPQQTLLTQPAVALPTSLSLSTPQPTAQITVSYPTPR |
| | | SSQQQTQPQKQRVFTGVVTKLHDTFGFVDEDVFFQLSAVKGKTPQ |
| | | VGDRVLVEATYNPNMPF |
| 28 | CARP-1: | PFKWNAQRIQTLPNQNQSQTQPLLKTPPAVLQPIAPQTTFGVQTQPQ |
| | 197- | PQSLLQAQISAASITPLLQTQPQPLLQQPQQKAGLLQPPVRIVSQPQP |
| | 454 | ARRLDPPSRFSGRNDRGDQVPNRKDDRSRERERERRRSRERSPQRK |
| | | RSRERSPRRERERSPRRVRRVVPRYTVQFSKFSLDCPSCDMMELRR |
| | | RYQNLYIPSDFFDAQFTWVDAFPLSRPFQLGNYCNFYVMHREVES |
| | | LEKNMAILDPPDADHLYSAKVMLMAS |
| 29 | CARP-1: | GERKEADGEQDEEEKDDGEAKEISTPTHWSKLDPKTMKVNDLRK |
| | 603- | ELESRALSSKGLKSQLIARLTKQLKVEEQKEEQKELEKSEKEEDED |
| | 898 | DDRKSEDDKEEEERKRQEEIERQRRERRYILPDEPAIIVHPNWAAK |
| | | SGKFDCSIMSLSVLLDYRLEDNKEHSFEVSLFAELFNEMLQRDFG |
| | | VRIYKSLLSLPEKEDKKEKDKKSKKDERKDKKEERDDETDEPKP |
| | | KRRKSGDDKDKKEDRDERKKEDKRKGDSKDDDETEEDNNQDE |
| | | YDPMEAEEAEDEEDDRDEEEMTKRDDKRD |
| 30 | CARP-1: | KRDINRYCKERPSKDKEKEKTQMITINRDLLMAFVYFDQSHCGYL |
| | 896- | LEKDLEEILYTLGLHLSRAQVKKLLNKVVLRESCFYRKLTDTSKD |
| | 1150 | EENHEESESLQEDMLGNRLLLPTPTVKQESKDVEENVGLIVYNGA |
| | | MVDVGSLLQKLEKSEKVRAEVEQKLQLLEEKTDEDEKTILNLENS |
| | | NKSLSGELREVKKDLSQLQENLKISENMSLQFENQMNKTIRNLST |
| | | VMDEIHTVLKKDNVKNEDKDQKSKENGASV |
| 31 | CARP-1: | MASPSMEDLYHKSCALAEDPQELRDGFQHPARLVKFLVGMKGK |
| | 452- | DEAMAIGGHWSPSLDGPDPEKDPSVLIKTAIRCCKALTGIDLSVC |
| | 625 | TQWYRFAEIRYHRPEETHKGRTVPAHVETVVLFFPDVWHCLPT |
| | | RSEWETLSRGYKQQLVEKLQGERKEADGEQDEEEKDDGEAKEI |
| 32 | CARP-1: | MASPSMEDLYHKSCALAEDPQELRDGFQHPARLVKFLVGMKGK |
| | 452- | DEAMAIGGHWSPSLDGPDPEKDPSVLIKTAIRCCKALTGIDLSVC |
| | 610 | TQWYRFAEIRYHRPEETHKGRTVPAHVETVVLFFPDVWHCLPTR |
| | | SEWETLSRGYKQQLVEKLQGERKEADG |
| 33 | CARP-1: | MASPSMEDLYHKSCALAEDPQELRDGFQHPARLVKFLVGMK |
| | 452- | GKDEAMAIGGHWSPSLDGPDPEKDPSVLIKTAIRCCKALTGID |
| | 552 | LSVCTQWYRFAEIRYHR |
| 34 | CARP-1: | RPEETHKGRTVPAHVETVVLFFPDVWHCLPTRSEWETLSRGY |
| | 552- | KQQLVEKLQGERKEADGEQDEEEKDDGEAKEISTPTHWSKL |
| | 640 | DPKTMK |
| 35 | CARP-1: | RPEETHKGRTVPAHVETVVLFFPDVWHCLPTRSEWETLSRGY |
| | 552- | KQQLVEKLQGERKEADGEQDEEEKDDGEAKEI |
| | 625 | |
| 36 | CARP-1: | RPEETHKGRTVPAHVETVVLFFPDVWHCLPTRSEWETLSRGYK |
| | 552- | QQLVEKLQGERKEADG |
| | 610 | |
| 37 | CARP-1: | LFFPDVWHCLPTRSEWETLSRGYKQQLVEK |
| | 571- | |
| | 600 | |
| 38 | CARP-1: | RGYKQQLVEKLQGERKEADGEQDEEEKDDG |
| | 591- | |
| | 620 | |
| 39 | NEMO: | SEEKRKLAQLQVAYHQLFQEYDNHIKSSVVGSERKRGMQLED |
| | 221-317 | LKQQLQQAEEALVAKQEVIDKLKEEAEQHKIVMETVPVLKAQ |
| | | ADIYKADFQAERQ |
| 40 | NEMO: | ETVPVLKAQADIYKADFQAERQAREKLAEKKELLQEQLEQLQ |
| | 296-419 | REYSKLKASCQESARIEDMRKRHVEVSQAPLPPAPAYLSSPLAL |
| | | PSQRRSPPEEPPDFCCPKCQYQAPDMDTLQIHVMECIE |
| 41 | CARP-1: | EQDEEEKDDGEAKEISTPTHWSKLDPKTMK |
| | 611- | |
| | 640 | |
| 42 | CARP-1: | WSKLDPKTMKVNDLRKELESRALSSKGLKS |
| | 631- | |
| | 660 | |
| 43 | NEMO: | MNRHLWKSQLCEMVQPSGGPAADQDVLGEESPLGKPAMLHLPSE |
| | human | QGAPETLQRCLEENQELRDAIRQSNQILRERCEELLHFQASQREEKE |
| | | FLMCKFQEARKLVERLGLEKLDLKRQKEQALREVEHLKRCQQQM |
| | | AEDKASVKAQVTSLLGELQESQSRLEAATKECQALEGRARAASEQ |
| | | ARQLESEREALQQQHSVQVDQLRMQGQSVEAALRMERQAASEEK |
| | | RKLAQLQVAYHQLFQEYDNHIKSSVVGSERKRGMQLEDLKQQLQ |
| | | QAEEALVAKQEVIDKLKEEAEQHKIVMETVPVLKAQADIYKADFQ |
| | | AERQAREKLAEKKELLQEQLEQLQREYSKLKASCQESARIEDMRK |
| | | RHVEVSQAPLPPAPAYLSSPLALPSQRRSPPEEPPDFCCPKCQYQAP |
| | | DMDTLQIHVMECIE |

Compounds

In some aspect, the compounds disclosed herein can be CARP-1-NEMO inhibitors. In some aspects, the invention relates to compounds useful in treating disorders associated with CARP-1 signaling including, but not limited to, cancer. In some aspects, the compounds described herein are useful in inhibiting cell cycle progression, cell growth, DNA repair, enhancing a chemotherapeutic response in a subject, reducing chemotherapeutic toxicity in a subject, reducing or preventing chemotherapeutic resistance in a cancer cell, inhibiting binding of NF-κB activating kinase IKK subunit γ (NEMO) to cell cycle and apoptosis regulatory protein (CARP)-1, reducing systemic levels of one or more cytokines in a subject, and enhancing the efficacy of radiotherapy and/or a chemotherapeutic agent.

Disclosed herein are compounds for administering to a subject. Disclosed herein are compounds for treating a subject with a cancer. Disclosed herein are also compounds that can be useful for inhibiting cell cycle progression, cell growth or DNA repair. The compounds disclosed herein can also be useful for enhancing a chemotherapeutic response in a subject. Further, the compounds disclosed herein can be useful for reducing chemotherapeutic toxicity in a subject. The compounds disclosed herein can also be useful reducing or preventing chemotherapeutic resistance in a cancer cell. The compounds disclosed herein can be useful for inhibiting binding of NF-κB activating kinase IKK subunit γ (NEMO) to cell cycle and apoptosis regulatory protein (CARP)-1. The compounds disclosed herein can be useful for reducing systemic levels of one or more cytokines in a subject. Further, the compounds disclosed herein can be useful for enhancing the efficacy of radiotherapy and/or a chemotherapeutic agent.

In some aspects, the disclosed compounds exhibit chemotherapeutic activity.

In some aspects, the compounds of the invention are useful in inhibiting CARP-1 NEMO in a mammal. In some aspects, the compounds of the invention are useful in inhibiting CARP-1 NEMO in at least one cell.

In some aspects, the compounds of the invention are useful in the treatment of cancer, as further described herein.

It is contemplated that each disclosed derivative can be optionally further substituted.

It is also contemplated that any one or more derivative can be optionally omitted from the invention. It is understood that a disclosed compound can be provided by the disclosed methods. It is also understood that the disclosed compounds can be employed in the disclosed methods of using.

Structure. In some aspects, disclosed are compounds having a structure represented by a formula:

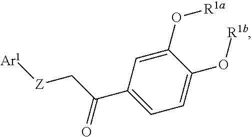

wherein Z is selected from —S(O)— and —SO₂—; wherein each of R^{1a} and R^{1b} is independently selected from hydrogen and C1-C4 alkyl, or wherein each of R^{1a} and R^{1b} are covalently bonded, and, together with the intermediate atoms, comprise a 6-membered heterocycle; or wherein each of R^{1a} and R^{1b} together comprise —CH₂—; and wherein Ar¹ is a structure having a formula selected from:

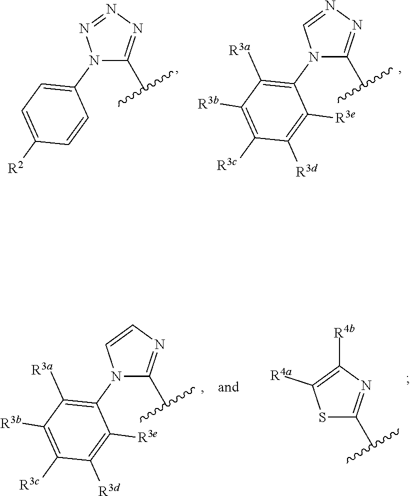

wherein R², when present, is C1-C4 alkyl; wherein each of R^{3a}, R^{3b}, R^{3c}, R^{3d}, and R^{3e}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar², provided that at least one of R^{4a} and R^{4b}, when present, is not hydrogen; and wherein Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; or wherein each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In some aspects, disclosed are compounds having a structure selected from:

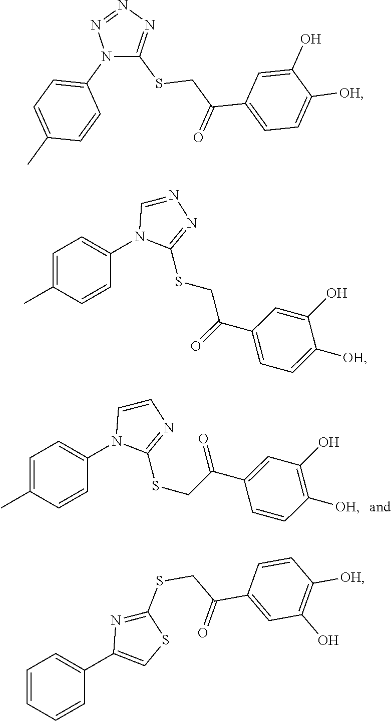

or a pharmaceutically acceptable salt thereof.

In some aspects, disclosed are compounds having a structure represented by a formula:

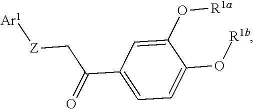

wherein Z is selected from —S—, —S(O)—, and —SO₂—; wherein each of R^{1a} and R^{1b} is independently selected from hydrogen and C1-C4 alkyl, or wherein each of R^{1a} and R^{1b} are covalently bonded, and, together with the intermediate atoms, comprise a 6-membered heterocycle; or wherein each of R^{1a} and R^{1b} together comprise —CH₂—; and wherein Ar¹ is a 5- to 10-membered heteroaryl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar²; and wherein Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar is thiazole, then Ar¹ is substituted by at least 1 group, provided that when Ar¹ is thiazole and Z is —S—, then each of R^{1a} and R^{1b} is hydrogen, provided that when Ar¹ is benzo[d]thiazole and Z is —S—, then each of R^{1a} and R^{1b} is hydrogen, and provided that when Ar¹ is tetrazole, then Ar¹ has a structure represented by a formula:

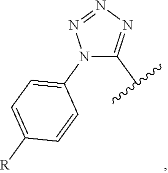

wherein R² is C1-C4 alkyl, and Z is —S(O)— or —SO₂—, or a pharmaceutically acceptable salt thereof.

In a further aspect, the compound has a structure represented by a formula selected from:

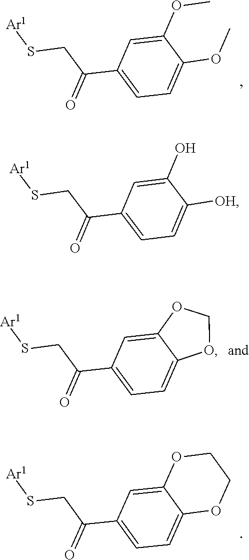

In a further aspect, the compound has a structure represented by a formula:

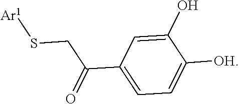

In a further aspect, the compound has a structure represented by a formula selected from:

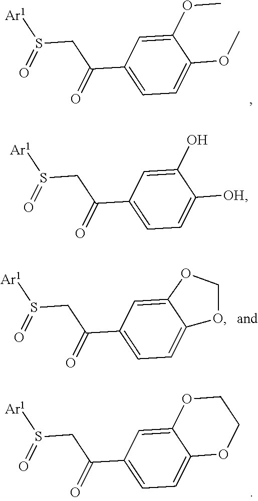

In a further aspect, the compound has a structure represented by a formula:

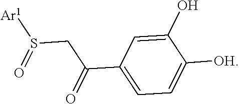

In a further aspect, the compound has a structure represented by a formula selected from:

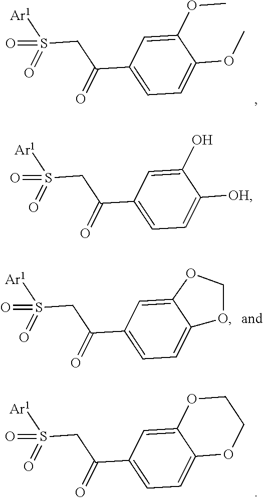

In a further aspect, the compound has a structure represented by a formula:

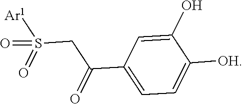

In a further aspect, the compound has a structure represented by a formula selected from:

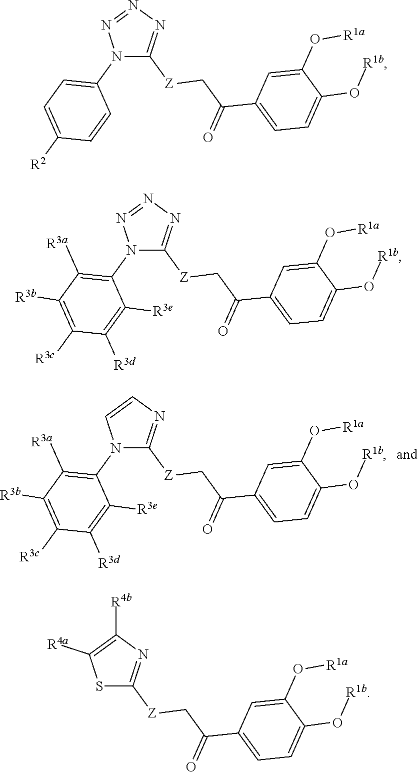

In a further aspect, the compound has a structure represented by a formula selected from:

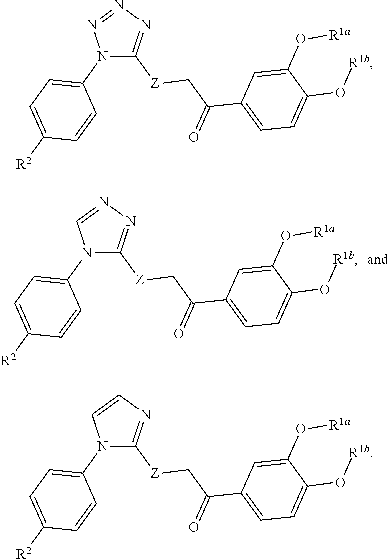

In a further aspect, the compound has a structure represented by a formula selected from:

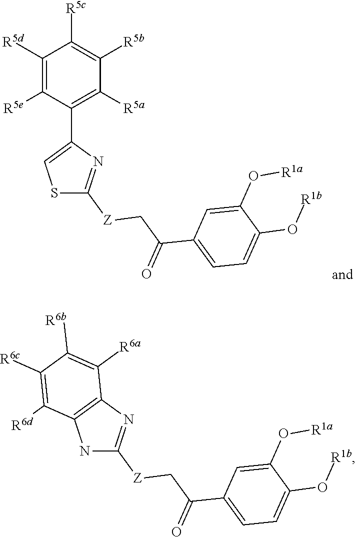

wherein each of R^{5a}, R^{5b}, R^{5c}, R^{5d}, and R^{5e}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least two of R^{5a}, R^{5b}, R^{5c}, R^{5d}, and R^{5e}, when present, is hydrogen; and wherein each of R^{6a}, R^{6b}, R^{6c}, and R^{6d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a}, R^{6b}, R^{6c}, and R^{6d}, when present, is hydrogen.

In a further aspect, the compound has a structure represented by a formula selected from:

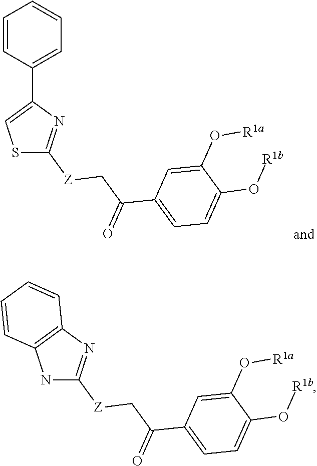

In a further aspect, the compound is selected from:

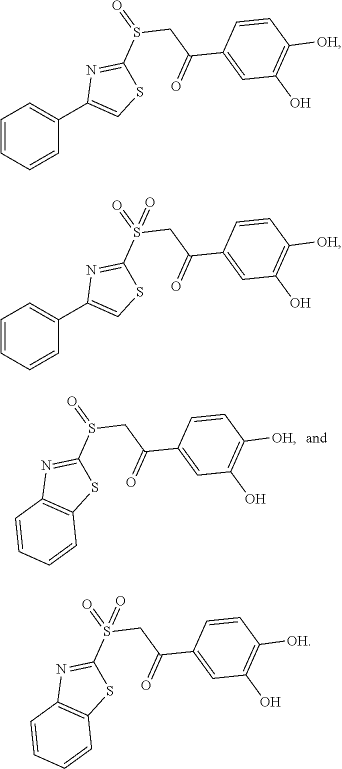 [SMILES: O=C(CS(=O)c1nc(-c2ccccc2)cs1)c1ccc(O)c(O)c1 ; O=C(CS(=O)(=O)c1nc(-c2ccccc2)cs1)c1ccc(O)c(O)c1 ; O=C(CS(=O)c1nc2ccccc2s1)c1ccc(O)c(O)c1 ; O=C(CS(=O)(=O)c1nc2ccccc2s1)c1ccc(O)c(O)c1]

In a further aspect, the compound is selected from:

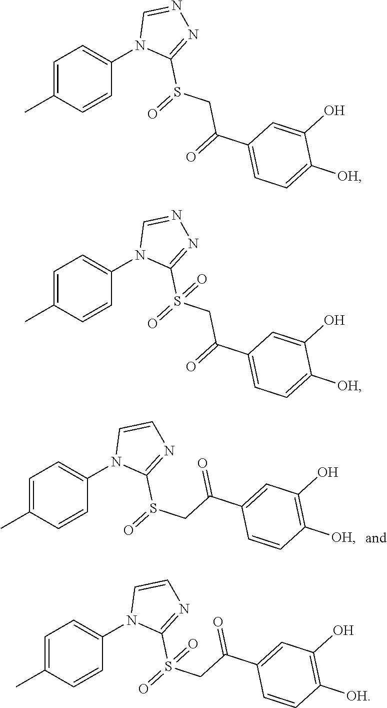 [SMILES: Cc1ccc(-n2cnnc2S(=O)CC(=O)c2ccc(O)c(O)c2)cc1 ; Cc1ccc(-n2cnnc2S(=O)(=O)CC(=O)c2ccc(O)c(O)c2)cc1 ; Cc1ccc(-n2ccnc2S(=O)CC(=O)c2ccc(O)c(O)c2)cc1 ; Cc1ccc(-n2ccnc2S(=O)(=O)CC(=O)c2ccc(O)c(O)c2)cc1]

Z Groups. In some aspects, Z is selected from —S(O)— and —SO₂—. In a further aspect, Z is —S(O)—. In a still further aspect, Z is —SO₂—.

In some aspects, Z is selected from —S—, —S(O)—, and —SO₂—. In a further aspect, Z is selected from —S— and —S(O)—. In a still further aspect, Z is selected from —S— and —SO₂—. In yet a further aspect, Z is —S—.

R^{1a} and R^{1b} Groups. In some aspects, each of R^{1a} and R^{1b} is independently selected from hydrogen and C1-C4 alkyl, or each of R^{1a} and R^{1b} are covalently bonded, and, together with the intermediate atoms, comprise a 6-membered heterocycle, or each of R^{1a} and R^{1b} together comprise —CH₂—.

In a further aspect, each of R^{1a} and R^{1b} is independently selected from hydrogen and C1-C4 alkyl. In a still further aspect, each of R^{1a} and R^{1b} is independently selected from hydrogen, methyl, ethyl, n-propyl, and isopropyl. In yet a further aspect, each of R^{1a} and R^{1b} is independently selected from hydrogen, methyl, and ethyl. In an even further aspect, each of R^{1a} and R^{1b} is independently selected from hydrogen and ethyl. In a still further aspect, each of R^{1a} and R^{1b} is independently selected from hydrogen and methyl. In yet a further aspect, each of R^{1a} and R^{1b} is hydrogen.

In a further aspect, each of R^{1a} and R^{1b} is independently C1-C4 alkyl. In a still further aspect, each of R^{1a} and R^{1b} is independently selected from methyl, ethyl, n-propyl, and isopropyl. In yet a further aspect, each of R^{1a} and R^{1b} is independently selected from methyl and ethyl. In an even further aspect, each of R^{1a} and R^{1b} is ethyl. In a still further aspect, each of R^{1a} and R^{1b} is methyl.

In a further aspect, each of R^{1a} and R^{1b} are covalently bonded, and, together with the intermediate atoms, comprise a 6-membered heterocycle. In a still further aspect, each of R^{1a} and R^{1b} are covalently bonded, and, together with the intermediate atoms, comprise a structure:

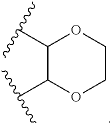 [SMILES: *C1OCCOC1*]

In a further aspect, each of R^{1a} and R^{1b} together comprise —CH₂—. Thus, in a still further aspect, each of R^{1a} and R^{1b} together comprise —CH₂—, and, together with the adjacent atoms, comprise a structure:

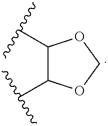 [SMILES: *C1OCOC1*]

R² Groups. In some aspects, R², when present, is C1-C4 alkyl. In a still further aspect, R², when present, is selected from methyl, ethyl, n-propyl, and isopropyl. In yet a further aspect, R², when present, is selected from methyl and ethyl. In an even further aspect, R², when present, is ethyl. In a still further aspect, R², when present, is methyl.

R^{3a}, R^{3b}, R^{3c}, R^{3d}, and R^{3e} Groups. In some aspects, each of R^{3a}, R^{3b}, R^{3c}, R^{3d}, and R^{3e}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{3a}, R^{3b}, R^{3c}, R^{3d}, and R^{3e}, when present, is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂H, —CH(CH₃)CH₂H, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{3a}, R^{3b}, R^{3c}, R^{3d}, and R^{3e}, when present, is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{3a}, R^{3b}, R^{3c}, R^{3d}, and R^{3e}, when present, is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{3a}, R^{3b}, R^{3c}, R^{3d}, and R^{3e}, when present, is independently selected from hydrogen, halogen, —CN, —NH₂, —OH, —NO₂, C1-C4 alkyl, and C2-C4 alkenyl. In a further aspect, each of R^{3a}, R^{3b}, R^{3c}, R^{3d}, and R^{3e}, when present, is independently selected from hydrogen, —F, —Cl, —CN, —NH₂, —OH, —NO₂, methyl, ethyl, n-propyl, isopropyl, ethenyl, and propenyl. In a still further aspect, each of R^{3a}, R^{3b}, R^{3c}, R^{3d}, and R^{3e}, when present, is independently selected from hydrogen, —F, —Cl, —CN, —NH₂, —OH, —NO₂, methyl, ethyl, and ethenyl. In yet a further aspect, each of R^{3a}, R^{3b}, R^{3c}, R^{3d}, and R^{3e}, when present, is independently selected from hydrogen, —F, —Cl, —CN, —NH₂, —OH, —NO₂, and methyl.

In various aspects, each of R^{3a}, R^{3b}, R^{3c}, R^{3d}, and R^{3e}, when present, is independently selected from hydrogen, C1-C4 alkyl, and C2-C4 alkenyl. In a further aspect, each of R^{3a}, R^{3b}, R^{3c}, R^{3d} and R^{3e}, when present, is independently selected from hydrogen, methyl, ethyl, n-propyl, isopropyl, ethenyl, and propenyl. In a still further aspect, each of R^{3a}, R^{3b}, R^{3c}, R^{3d}, and R^{3e}, when present, is independently selected from hydrogen, methyl, ethyl, and ethenyl. In yet a further aspect, each of R^{3a}, R^{3b}, R^{3c}, R^{3d}, and R^{3e}, when present, is independently selected from hydrogen and methyl.

In various aspects, each of R^{3a}, R^{3b}, R^{3c}, R^{3d}, and R^{3e}, when present, is independently selected from hydrogen, halogen, —CN, —NH₂, —OH, —NO₂, C1-C4 haloalkyl, and C1-C4 cyanoalkyl. In a further aspect, each of R^{3a}, R^{3b}, R^{3c}, R^{3d}, and R^{3e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{3a}, R^{3b}, R^{3c}, R^{3d}, and R^{3e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{3a}, R^{3b}, R^{3c}, R^{3d}, and R^{3e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{3a}, R^{3b}, R^{3c}, R^{3d}, and R^{3e}, when present, is independently selected from hydrogen, C1-C4 haloalkyl, and C1-C4 cyanoalkyl. In a further aspect, each of R^{3a}, R^{3b}, R^{3c}, R^{3d}, and R^{3e}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{3a}, R^{3b}, R^{3c}, R^{3d}, and R^{3e}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{3a}, R^{3b}, R^{3c}, R^{3d}, and R^{3e}, when present, is independently selected from hydrogen, —CCl₃, —CF₃, —CHCl₂, —CHF₂, —CH₂Cl, —CH₂F, and —CH₂CN.

In various aspects, each of R^{3a}, R^{3b}, R^{3c}, R^{3d}, and R^{3e}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{3a}, R^{3b}, R^{3c}, R^{3d}, and R^{3e}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{3a}, R^{3b}, R^{3c}, R^{3d}, and R^{3e}, when present, is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{3a}, R^{3b}, R^{3c}, R^{3d} and R^{3e}, when present, is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{3a}, R^{3b}, R^{3c}, R^{3d}, and R^{3e}, when present, is independently selected from hydrogen, C1-C4 hydroxyalkyl, C1-C4 haloalkoxy, and C1-C4 alkoxy. In a further aspect, each of R^{3a}, R^{3b}, R^{3c}, R^{3d}, and R^{3e}, when present, is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{3a}, R^{3b}, R^{3c}, R^{3d}, and R^{3e}, when present, is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{3a}, R^{3b}, R^{3c}, R^{3d}, and R^{3e}, when present, is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{3a}, R^{3b}, R^{3c}, R^{3d}, and R^{3e}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{3a}, R^{3b}, R^{3c}, R^{3d}, and R^{3e}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{3a}, R^{3b}, R^{3c}, R^{3d}, and R^{3e}, when present, is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, and —CH₂CH₂NH₂. In yet a further aspect, each of R^{3a}, R^{3b}, R^{3c}, R^{3d}, and R^{3e}, when present, is independently selected from hydrogen, —F, —Cl, —CN, —NH₂, —OH, —NO₂, —NHCH₃, —N(CH₃)₂, and —CH₂NH₂.

In various aspects, each of R^{3a}, R^{3b}, R^{3c}, R^{3d}, and R^{3e}, when present, is independently selected from hydrogen, C1-C4 alkylamino, (C1-C4)(C1-C4) dialkylamino, and C1-C4 aminoalkyl. In a further aspect, each of R^{3a}, R^{3b}, R^{3c}, R^{3d}, and R^{3e}, when present, is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{3a}, R^{3b}, R^{3c}, R^{3d}, and R^{3e}, when present, is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{3a}, R^{3b}, R^{3c}, R^{3d}, and R^{3e}, when present, is independently selected from hydrogen, —NHCH₃, —N(CH₃)₂, and —CH₂NH₂.

In various aspects, each of R^{3a}, R^{3b}, R^{3c}, R^{3d}, and R^{3e}, when present, is independently selected from hydrogen and halogen. In a further aspect, each of R^{3a}, R^{3b}, R^{3c}, R^{3d}, and R^{3e}, when present, is independently selected from hydrogen, —F, —Cl, and —Br. In a still further aspect, each of R^{3a}, R^{3b}, R^{3c}, R^{3d}, and R^{3e}, when present, is independently selected from hydrogen, —F, and —Cl. In yet a further aspect, each of R^{3a}, R^{3c}, R^{3d}, and R^{3e}, when present, is independently selected from hydrogen and —Cl. In an even further aspect, each of R^{3a}, R^{3b}, R^{3c}, R^{3d}, and R^{3e}, when present, is independently selected from hydrogen and —F.

In various aspects, at least one of R^{3a}, R^{3b}, R^{3c}, R^{3d}, and R^{3e}, when present, is hydrogen. In a further aspect, at least two of R^{3a}, R^{3b}, R^{3c}, R^{3d}, and R^{3e}, when present, is hydrogen. In a still further aspect, at least three of R^{3a}, R^{3b}, R^{3c}, R^{3d}, and R^{3e}, when present, is hydrogen. In yet a further aspect, at least four of R^{3a}, R^{3b}, R^{3c}, R^{3d}, and R^{3e}, when present, is hydrogen. In an even further aspect, each of R^{3a}, R^{3b}, R^{3c}, R^{3d}, and R^{3e}, when present, is hydrogen.

In various aspects, each of R^{3a}, R^{3b}, R^{3d}, and R^{3e}, when present, is hydrogen, and R³, when present, is selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{3a}, R^{3b}, R^{3d}, and R^{3e}, when present, is hydrogen, and R^{3e}, when present, is selected from —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{3a}, R^{3b}, R^{3d}, and R^{3e}, when present, is hydrogen, and R^{3e}, when present, is selected from —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{3a}, R^{3b}, R^{3d}, and R^{3e}, when present, is hydrogen, and R^{3e}, when present, is selected from —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{3a}, R^{3b}, R^{3d}, and R^{3e}, when present, is hydrogen, and R³, when present, is selected from halogen, —CN, —NH₂, —OH, —NO₂, C1-C4 alkyl, and C2-C4 alkenyl. In a further aspect, each of R^{3a}, R^{3b}, R^{3d}, and R^{3e}, when present, is hydrogen, and R^{3e}, when present, is selected from —F, —Cl, —CN, —NH₂, —OH, —NO₂, methyl, ethyl, n-propyl, isopropyl, ethenyl, and propenyl. In a still further aspect, each of R^{3a}, R^{3b}, R^{3d}, and R^{3e}, when present, is hydrogen, and R^{3e}, when present, is selected from —F, —Cl, —CN, —NH₂, —OH, —NO₂, methyl, ethyl, and ethenyl. In yet a further aspect, each of R^{3a}, R^{3d}, and R^{3e}, when present, is hydrogen, and R^{3e}, when present, is selected from —F, —Cl, —CN, —NH₂, —OH, —NO₂, and methyl.

In various aspects, each of R^{3a}, R^{3b}, R^{3d}, and R^{3e}, when present, is hydrogen, and R³, when present, is selected from C1-C4 alkyl and C2-C4 alkenyl. In a further aspect, each of R^{3a}, R^{3b}, R^{3d} and R^{3e}, when present, is hydrogen, and R^{3e}, when present, is selected from methyl, ethyl, n-propyl, isopropyl, ethenyl, and propenyl. In a still further aspect, each of R^{3a}, R^{3b}, R^{3d} and R^{3e}, when present, is hydrogen, and R^{3e}, when present, is selected from methyl, ethyl, and ethenyl.

In various aspects, each of R^{3a}, R^{3b}, R^{3d}, and R^{3e}, when present, is hydrogen, and R³, when present, is C1-C4 alkyl. Ina further aspect, each of R^{3a}, R^{3b}, R^{3d}, and R^{3e}, when present, is hydrogen, and R³, when present, is selected from methyl, ethyl, n-propyl, and isopropyl. In a still further aspect, each of R^{3a}, R^{3b}, R^{3d}, and R^{3e}, when present, is hydrogen, and R³, when present, is selected from methyl and ethyl. In yet a further aspect, each of R^{3a}, R^{3b}, R^{3d}, and R^{3e}, when present, is hydrogen, and R^{3c}, when present, is methyl.

R^{4a} and R^{4b} Groups. In some aspects, each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar², provided that at least one of R^{4a} and R^{4b}, when present, is not hydrogen, or each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In a further aspect, each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, each of R^{4a} and R^{4b}, when present, is independently selected from hydrogen, Ar², —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{4a} and R^{4b}, when present, is independently selected from hydrogen, Ar², —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{4a} and R^{4b}, when present, is independently selected from hydrogen, Ar², —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{4a} and R^{4b}, when present, is independently selected from hydrogen, Ar², halogen, —CN, —NH₂, —OH, —NO₂, C1-C4 alkyl, and C2-C4 alkenyl. In a further aspect, each of R^{4a} and R^{4b}, when present, is independently selected from hydrogen, Ar², —F, —Cl, —CN, —NH₂, —OH, —NO₂, methyl, ethyl, n-propyl, isopropyl, ethenyl, and propenyl. In a still further aspect, each of R^{4a} and R^{4b}, when present, is independently selected from hydrogen, Ar², —F, —Cl, —CN, —NH₂, —OH, —NO₂, methyl, ethyl, and ethenyl.

In yet a further aspect, each of R^{4a} and R^{4b}, when present, is independently selected from hydrogen, Ar², —F, —Cl, —CN, —NH₂, —OH, —NO₂, and methyl.

In various aspects, each of R^{4a} and R^{4b}, when present, is independently selected from hydrogen, Ar², C1-C4 alkyl, and C2-C4 alkenyl. In a further aspect, each of R^{4a} and R^{4b}, when present, is independently selected from hydrogen, methyl, ethyl, n-propyl, isopropyl, ethenyl, and propenyl. In a still further aspect, each of R^{4a} and R^{4b}, when present, is independently selected from hydrogen, Ar², methyl, ethyl, and ethenyl. In yet a further aspect, each of R^{4a} and R^{4b}, when present, is independently selected from hydrogen, Ar², and methyl.

In various aspects, each of R^{4a} and R^{4b}, when present, is independently selected from hydrogen, Ar², halogen, —CN, —NH₂, —OH, —NO₂, C1-C4 haloalkyl, and C1-C4 cyanoalkyl.

In a further aspect, each of R^{4a} and R^{4b}, when present, is independently selected from hydrogen, Ar², —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{4a} and R^{4b}, when present, is independently selected from hydrogen, Ar², —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{4a} and R^{4b}, when present, is independently selected from hydrogen, Ar², —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{4a} and R^{4b}, when present, is independently selected from hydrogen, Ar², C1-C4 haloalkyl, and C1-C4 cyanoalkyl. Ina further aspect, each of R^{4a} and R^{4b}, when present, is independently selected from hydrogen, Ar², —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{4a} and R^{4b}, when present, is independently selected from hydrogen, Ar², —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{4a} and R^{4b}, when present, is independently selected from hydrogen, Ar², —CCl₃, —CF₃, —CHCl₂, —CHF₂, —CH₂Cl, —CH₂F, and —CH₂CN.

In various aspects, each of R^{4a} and R^{4b}, when present, is independently selected from hydrogen, Ar², halogen, —CN, —NH₂, —OH, —NO₂, C1-C4 hydroxyalkyl, C1-C4 haloalkoxy, and C1-C4 alkoxy. In a further aspect, each of R^{4a} and R^{4b}, when present, is independently selected from hydrogen, Ar², —F, —Cl, —CN, —NH₂, —OH, —NO₂, —CH₂OH, —CH₂CH₂OH, —CH₂CH₂CH₂H, —CH(CH₃)CH₂H, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{4a} and R^{4b}, when present, is independently selected from hydrogen, Ar², —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{4a} and R^{4b}, when present, is independently selected from hydrogen, Ar², —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{4a} and R^{4b}, when present, is independently selected from hydrogen, Ar², C1-C4 hydroxyalkyl, C1-C4 haloalkoxy, and C1-C4 alkoxy. In a further aspect, each of R^{4a} and R^{4b}, when present, is independently selected from hydrogen, Ar², —CH₂OH, —CH₂CH₂OH, —CH₂CH₂CH₂OH, —CH(CH₃)CH₂H, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{4a} and R^{4b}, when present, is independently selected from hydrogen, Ar², —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{4a} and R^{4b}, when present, is independently selected from hydrogen, Ar², —CH₂OH, —OCCl₃, —OCF₃, —OCHCl₂, —OCHF₂, —OCH₂Cl, —OCH₂F, and —OCH₃.

In various aspects, each of R^{4a} and R^{4b}, when present, is independently selected from hydrogen, Ar², halogen, —CN, —NH₂, —OH, —NO₂, C1-C4 alkylamino, (C1-C4)(C1-C4) dialkylamino, and C1-C4 aminoalkyl. In a further aspect, each of R^{4a} and R^{4b}, when present, is independently selected from hydrogen, Ar², —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{4a} and R^{4b}, when present, is independently selected from hydrogen, Ar², —F, —Cl, —CN, —NH₂, —OH, —NO₂, —NHCH₃, —NHCH₂CH₃, —N(CH₃)₂, —N(CH₃)CH₂CH₃, —CH₂NH₂, and —CH₂CH₂NH₂. In yet a further aspect, each of R^{4a} and R^{4b}, when present, is independently selected from hydrogen, Ar², —F, —Cl, —CN, —NH₂, —OH, —NO₂, —NHCH₃, —N(CH₃)₂, and —CH₂NH₂.

In various aspects, each of R^{4a} and R^{4b}, when present, is independently selected from hydrogen, Ar², CT-C4 alkylamino, (C1-C4)(C1-C4) dialkylamino, and C1-C4 aminoalkyl. In a further aspect, each of R^{4a} and R^{4b}, when present, is independently selected from hydrogen, Ar², —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{4a} and R^{4b}, when present, is independently selected from hydrogen, Ar², —NHCH₃, —NHCH₂CH₃, —N(CH₃)₂, —N(CH₃)CH₂CH₃, —CH₂NH₂, and —CH₂CH₂NH₂. In yet a further aspect, each of R^{4a} and R^{4b}, when present, is independently selected from hydrogen, Ar², —NHCH₃, —N(CH₃)₂, and —CH₂NH₂.

In various aspects, each of R^{4a} and R^{4b}, when present, is independently selected from hydrogen, Ar², and halogen. In a further aspect, each of R^{4a} and R^{4b}, when present, is independently selected from hydrogen, Ar², —F, —Cl, and —Br. In a still further aspect, each of R^{4a} and R^{4b}, when present, is independently selected from hydrogen, Ar², —F, and —Cl. In yet a further aspect, each of R^{4a} and R^{4b}, when present, is independently selected from hydrogen Ar², and —Cl. In an even further aspect, each of R^{4a} and R^{4b}, when present, is independently selected from hydrogen, Ar², and —F.

In various aspects, each of R^{4a} and R^{4b}, when present, is independently selected from hydrogen and Ar². In a further aspect, R^{4a}, when present, is hydrogen, and R^{4b}, when present, is Ar². In a still further aspect, R^{4b}, when present, is hydrogen, and R^{4a}, when present, is Ar².

In various aspects, one of R^{4a} and R^{4b}, when present, is Ar², and one of R^{4a} and R^{4b}, when present, is selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, one of R^{4a} and R^{4b}, when present, is Ar², and one of R^{4a} and R^{4b}, when present, is selected from hydrogen, Ar², —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, one of R^{4a} and R^{4b}, when present, is Ar², and one of R^{4a} and R^{4b}, when present, is selected from hydrogen, Ar², —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, one of R^{4a} and R^{4b}, when present, is Ar², and one of R^{4a} and R^{4b}, when present, is selected from hydrogen, Ar², —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In a further aspect, each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{4a} and R^{4b}, when present, are covalently bonded and, together with the intermediate atoms, comprise a 6-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, each of R^{4a} and R^{4b}, when present, are covalently bonded and, together with the intermediate atoms, comprise a 6-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, each of R^{4a} and R^{4b}, when present, are covalently bonded and, together with the intermediate atoms, comprise a 6-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, each of R^{4a} and R^{4b}, when present, are covalently bonded and, together with the intermediate atoms, comprise an unsubstituted 6-membered aryl.

R^{5a}, R^{5b}, R^{5c}, R^{5d}, and R^{5e} Groups. In some aspects, each of R^{5a}, R^{5b}, R^{5c}, R^{5d}, and R^{5c}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least two of R^{5a}, R^{5b}, R^{5c}, R^{5d}, and R^{5e}, when present, is hydrogen. In a further aspect, each of R^{5a}, R^{5b}, R^{5c}, R^{5d}, and R^{5e}, when present, is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, R^{5c}, R^{5d}, and R^{5e}, when present, is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, R^{5c}, R^{5d}, and R^{5e}, when present, is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{5a}, R^{5b}, R^{5c}, R^{5d}, and R^{5e}, when present, is independently selected from hydrogen, halogen, —CN, —NH₂, —OH, —NO₂, C1-C4 alkyl, and C2-C4 alkenyl. In a further aspect, each of R^{5a}, R^{5b}, R^{5c}, R^{5d}, and R^{5e}, when present, is independently selected from hydrogen, —F, —Cl, —CN, —NH₂, —OH, —NO₂, methyl, ethyl, n-propyl, isopropyl, ethenyl, and propenyl. In a still further aspect, each of R^{5a}, R^{5b}, R^{5c}, R^{5d}, and R^{5e}, when present, is independently selected from hydrogen, —F, —Cl, —CN, —NH₂, —OH, —NO₂, methyl, ethyl, and ethenyl. In yet a further aspect, each of R^{5a}, R^{5b}, R^{5c}, R^{5d}, and R^{5e}, when present, is independently selected from hydrogen, —F, —Cl, —CN, —NH₂, —OH, —NO₂, and methyl.

In various aspects, each of R^{5a}, R^{5b}, R^{5c}, R^{5d}, and R^{5e}, when present, is independently selected from hydrogen, C1-C4 alkyl, and C2-C4 alkenyl. In a further aspect, each of R^{5a}, R^{5b}, R^{5c}, R^{5d}, and R^{5e}, when present, is independently selected from hydrogen, methyl, ethyl, n-propyl, isopropyl, ethenyl, and propenyl. In a still further aspect, each of R^{5a}, R^{5b}, R^{5c}, R^{5d}, and R^{5e}, when present, is independently selected from hydrogen, methyl, ethyl, and ethenyl. In yet a further aspect, each of R^{5a}, R^{5b}, R^{5c}, R^{5d}, and R^{5e}, when present, is independently selected from hydrogen and methyl.

In various aspects, each of R^{5a}, R^{5b}, R^{5c}, R^{5d}, and R^{5e}, when present, is independently selected from hydrogen, halogen, —CN, —NH₂, —OH, —NO₂, C1-C4 haloalkyl, and C1-C4 cyanoalkyl. In a further aspect, each of R^{5a}, R^{5b}, R^{5c}, R^{5d}, and R^{5e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, R^{5c}, R^{5d}, and R^{5e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, R^{5c}, R^{5d}, and R^{5e}, when present, is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{5a}, R^{5b}, R^{5c}, R^{5d}, and R^{5e}, when present, is independently selected from hydrogen, C1-C4 haloalkyl, and C1-C4 cyanoalkyl. In a further aspect, each of R^{5a}, R^{5b}, R^{5c}, R^{5d}, and R^{5e}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{5a}, R^{5b}, R^{5c}, R^{5d}, and R^{5e}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{5a}, R^{5b}, R^{5c}, R^{5d}, and R^{5e}, when present, is independently selected from hydrogen, —CCl₃, —CF₃, —CHCl₂, —CHF₂, —CH₂Cl, —CH₂F, and —CH₂CN.

In various aspects, each of R^{5a}, R^{5b}, R^{5c}, R^{5d}, and R^{5e}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{5a}, R^{5b}, R^{5c}, R^{5d}, and R^{5e}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂H, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, R^{5c}, R^{5d}, and R^{5e}, when present, is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5c}, R^{5b}, R^{5c}, R^{5d}, and R^{5e}, when present, is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, R^{5c}, R^{5d}, and R^{5e}, when present, is independently selected from hydrogen, C1-C4 hydroxyalkyl, C1-C4 haloalkoxy, and C1-C4 alkoxy. In a further aspect, each of R^{5a}, R^{5b}, R^{5c}, R^{5d}, and R^{5e}, when present, is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{5a}, R^{5b}, R^{5c}, R^{5d}, and R^{5e}, when present, is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{5a}, R^{5b}, R^{5c}, R^{5d}, and R^{5e}, when present, is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{5a}, R^{5b}, R^{5c}, R^{5d}, and R^{5e}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{5a}, R^{5b}, R^{5c}, R^{5d}, and R^{5e}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, R^{5c}, R^{5d}, and R^{5e}, when present, is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, R^{5c}, R^{5d}, and R^{5e}, when present, is independently selected from hydrogen, —F, —Cl, —CN, —NH₂, —OH, —NO₂, —NHCH₃, —N(CH₃)₂, and —CH₂NH₂.

In various aspects, each of R^{5a}, R^{5b}, R^{5c}, R^{5d}, and R^{5e}, when present, is independently selected from hydrogen, C1-C4 alkylamino, (C1-C4)(C1-C4) dialkylamino, and C1-C4 aminoalkyl. In a further aspect, each of R^{5a}, R^{5b}, R^{5c}, R^{5d}, and R^{5e}, when present, is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{5a}, R^{5b}, R^{5c}, R^{5d}, and R^{5e}, when present, is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{5a}, R^{5b}, R^{5c}, R^{5d}, and R^{5e}, when present, is independently selected from hydrogen, —NHCH₃, —N(CH₃)₂, and —CH₂NH₂.

In various aspects, each of R^{5a}, R^{5b}, R^{5c}, R^{5d}, and R^{5e}, when present, is independently selected from hydrogen and halogen. In a further aspect, each of R^{5a}, R^{5b}, R^{5c}, R^{5d}, and R^{5e}, when present, is independently selected from hydrogen, —F, —Cl, and —Br. In a still further aspect, each of R^{5a}, R^{5b}, R^{5c}, R^{5d}, and R^{5e}, when present, is independently selected from hydrogen, —F, and —Cl. In yet a further aspect, each of R^{5a}, R^{5b}, R^{5c}, R^{5d}, and R^{5e}, when present, is independently selected from hydrogen and —Cl. In an even further aspect, each of R^{5a}, R^{5b}, R^{5c}, R^{5d}, and R^{5e}, when present, is independently selected from hydrogen and —F.

In various aspects, each of R^{5a}, R^{5b}, R^{5c}, R^{5d}, and R^{5e}, when present, is hydrogen.

R^{6a}, R^{6b}, R^{6c}, and R^{6d} Groups. In some aspects, each of R^{6a}, R^{6b}, R^{6c}, and R^{6d}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that at least one of R^{6a}, R^{6b}, R^{6c}, and R^{6d}, when present, is hydrogen. In a further aspect, each of R^{6a}, R^{6b}, R^{6c}, and R^{6d}, when present, is independently selected from hydrogen, —F, —Cl, —CN, —NH₂, —OH, —NO₂, methyl, ethyl, n-propyl, isopropyl, ethenyl, propenyl, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{6a}, R^{6b}, R^{6c}, and R^{6d}, when present, is independently selected from hydrogen, —F, —Cl, —CN, —NH₂, —OH, —NO₂, methyl, ethyl, ethenyl, —CCl₃, —CF₃, —CHCl₂, —CHF₂, —CH₂Cl, —CH₂F, —CH₂CH₂Cl, —CH₂CH₂F, —CH₂CN, —CH₂CH₂CN, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{6a}, R^{6b}, R^{6c}, and R^{6d}, when present, is independently selected from hydrogen, —F, —Cl, —CN, —NH₂, —OH, —NO₂, methyl, —CCl₃, —CF₃, —CHCl₂, —CHF₂, —CH₂Cl, —CH₂F, —CH₂CN, —CH₂OH, —OCCl₃, —OCF₃, —OCHCl₂, —OCHF₂, —OCH₂Cl, —OCH₂F, —OCH₃, —NHCH₃, —N(CH₃)₂, and —CH₂NH₂.

In various aspects, each of R^{6a}, R^{6b}, R^{6c}, and R^{6d}, when present, is independently selected from hydrogen, halogen, —CN, —NH₂, —OH, —NO₂, C1-C4 alkyl, and C2-C4 alkenyl. In a further aspect, each of R^{6a}, R^{6b}, R^{6c}, and R^{6d}, when present, is independently selected from hydrogen, —F, —Cl, —CN, —NH₂, —OH, —NO₂, methyl, ethyl, n-propyl, isopropyl, ethenyl, and propenyl. In a still further aspect, each of R^{6a}, R^{6b}, R^{6c}, and R^{6d}, when present, is independently selected from hydrogen, —F, —Cl, —CN, —NH₂, —OH, —NO₂, methyl, ethyl, and ethenyl. In yet a further aspect, each of R^{6a}, R^{6b}, R^{6c}, and R^{6d}, when present, is independently selected from hydrogen, —F, —Cl, —CN, —NH₂, —OH, —NO₂, and methyl.

In various aspects, each of R^{6a}, R^{6b}, R^{6c}, and R^{6d}, when present, is independently selected from hydrogen, C1-C4 alkyl, and C2-C4 alkenyl. In a further aspect, each of R^{6a}, R^{6b}, R^{6c}, and R^{6d}, when present, is independently selected from hydrogen, methyl, ethyl, n-propyl, isopropyl, ethenyl, and propenyl. In a still further aspect, each of R^{6a}, R^{6b}, R^{6c}, and R^{6d}, when present, is independently selected from hydrogen, methyl, ethyl, and ethenyl. In yet a further aspect, each of R^{6a}, R^{6b}, R^{6c}, and R^{6d}, when present, is independently selected from hydrogen and methyl.

In various aspects, each of R^{6a}, R^{6b}, R^{6c}, and R^{6d}, when present, is independently selected from hydrogen, halogen, —CN, —NH₂, —OH, —NO₂, C1-C4 haloalkyl, and C1-C4 cyanoalkyl. In a further aspect, each of R^{6a}, R^{6b}, R^{6c}, and R^{6d}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{6a}, R^{6b}, R^{6c}, and R^{6d}, when present, is independently selected from hydrogen, —F, —Cl, —CN, —NH₂, —OH, —NO₂, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{6a}, R^{6b}, R^{6c}, and R^{6d}, when present, is independently selected from hydrogen, —F, —Cl, —CN, —NH₂, —OH, —NO₂, —CCl₃, —CF₃, —CHCl₂, —CHF₂, —CH₂Cl, —CH₂F, and —CH₂CN.

In various aspects, each of R^{6a}, R^{6b}, R^{6c}, and R^{6d}, when present, is independently selected from hydrogen, C1-C4 haloalkyl, and C1-C4 cyanoalkyl. In a further aspect, each of R^{6a}, R^{6b}, R^{6c}, and R^{6d}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CH₂CH₂Cl, —CH₂CH₂CH₂F, —CH(CH₃)CH₂Cl, —CH(CH₃)CH₂F, —CH₂CN, —CH₂CH₂CN, —CH₂CH₂CH₂CN, and —CH(CH₃)CH₂CN. In a still further aspect, each of R^{6a}, R^{6b}, R^{6c}, and R^{6d}, when present, is independently selected from hydrogen, —CCl₃, —CF₃, —CHCl₂, —CHF₂, —CH₂Cl, —CH₂F, —CH₂CH₂Cl, —CH₂CH₂F, —CH₂CN, and —CH₂CH₂CN. In yet a further aspect, each of R^{6a}, R^{6b}, R^{6c}, and R^{6d}, when present, is independently selected from hydrogen, —CCl₃, —CF₃, —CHCl₂, —CHF₂, —CH₂Cl, —CH₂F, and —CH₂CN.

In various aspects, each of R^{6a}, R^{6b}, R^{6c}, and R^{6d}, when present, is independently selected from hydrogen, halogen, —CN, —NH₂, —OH, —NO₂, C1-C4 hydroxyalkyl, C1-C4 haloalkoxy, and C1-C4 alkoxy. In a further aspect, each of R^{6a}, R^{6b}, R^{6c}, and R^{6d}, when present, is independently selected from hydrogen, —F, —Cl, —CN, —NH₂, —OH, —NO₂, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{6a}, R^{6b}, R^{6c}, and R^{6d}, when present, is independently selected from hydrogen, —F, —Cl, —CN, —NH₂, —OH, —NO₂, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{6a}, R^{6b}, R^{6c}, and R^{6d}, when present, is independently selected from hydrogen, —F, —Cl, —CN, —NH₂, —OH, —NO₂, —CH₂OH, —OCCl₃, —OCF₃, —OCHCl₂, —OCHF₂, —OCH₂Cl, —OCH₂F, and —OCH₃.

In various aspects, each of R^{6a}, R^{6b}, R^{6c}, and R^{6d}, when present, is independently selected from hydrogen, C1-C4 hydroxyalkyl, C1-C4 haloalkoxy, and C1-C4 alkoxy. In a further aspect, each of R^{6a}, R^{6b}, R^{6c}, and R^{6d}, when present, is independently selected from hydrogen, —CH₂OH, —CH₂CH₂OH, —CH₂CH₂CH₂OH, —CH(CH₃)CH₂OH, —OCCl₃, —OCF₃, —OCHCl₂, —OCHF₂, —OCH₂Cl, —OCH₂F, —OCH₂CH₂Cl, —OCH₂CH₂F, —OCH₂CH₂CH₂Cl, —OCH₂CH₂CH₂F, —OCH(CH₃)CH₂Cl, —OCH(CH₃)CH₂F, —OCH₃, —OCH₂CH₃, —OCH₂CH₂CH₃, and —OCH(CH₃)₂. In a still further aspect, each of R^{6a}, R^{6b}, R^{6c}, and R^{6d}, when present, is independently selected from hydrogen, —CH₂OH, —CH₂CH₂OH, —OCCl₃, —OCF₃, —OCHCl₂, —OCHF₂, —OCH₂Cl, —OCH₂F, —OCH₂CH₂Cl, —OCH₂CH₂F, —OCH₃, and —OCH₂CH₃. In yet a further aspect, each of R^{6a}, R^{6b}, R^{6c}, and R^{6d}, when present, is independently selected from hydrogen, —CH₂OH, —OCCl₃, —OCF₃, —OCHCl₂, —OCHF₂, —OCH₂Cl, —OCH₂F, and —OCH₃.

In various aspects, each of R^{6a}, R^{6b}, R^{6c}, and R^{6d}, when present, is independently selected from hydrogen, halogen, —CN, —NH₂, —OH, —NO₂, C1-C4 alkylamino, (C1-C4)(C1-C4) dialkylamino, and C1-C4 aminoalkyl. In a further aspect, each of R^{6a}, R^{6b}, R^{6c}, and R^{6d}, when present, is independently selected from hydrogen, —F, —Cl, —CN, —NH₂, —OH, —NO₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{6a}, R^{6b}, R^{6c}, and R^{6d}, when present, is independently selected from hydrogen, —F, —Cl, —CN, —NH₂, —OH, —NO₂, —NHCH₃, —NHCH₂CH₃, —N(CH₃)₂, —N(CH₃)CH₂CH₃, —CH₂NH₂, and —CH₂CH₂NH₂. In yet a further aspect, each of R^{6a}, R^{6b}, R^{6c}, and R^{6d}, when present, is independently selected from hydrogen, —F, —Cl, —CN, —NH₂, —OH, —NO₂, —NHCH₃, —N(CH₃)₂, and —CH₂NH₂.

In various aspects, each of R^{6a}, R^{6b}, R^{6c}, and R^{6d}, when present, is independently selected from hydrogen, C1-C4 alkylamino, (C1-C4)(C1-C4) dialkylamino, and C1-C4 aminoalkyl. In a further aspect, each of R^{6a}, R^{6b}, R^{6c}, and R^{6d}, when present, is independently selected from hydrogen, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{6a}, R^{6b}, R^{6c}, and R^{6d}, when present, is independently selected from hydrogen, —NHCH₃, —NHCH₂CH₃, —N(CH₃)₂, —N(CH₃)CH₂CH₃, —CH₂NH₂, and —CH₂CH₂NH₂. In yet a further aspect, each of R^{6a}, R^{6b}, R^{6c}, and R^{6d}, when present, is independently selected from hydrogen, —NHCH₃, —N(CH₃)₂, and —CH₂NH₂.

In various aspects, each of R^{6a}, R^{6b}, R^{6c}, and R^{6d}, when present, is independently selected from hydrogen and halogen. In a further aspect, each of R^{6a}, R^{6b}, R^{6c}, and R^{6d}, when present, is independently selected from hydrogen, —F, —Cl, and —Br. In a still further aspect, each of R^{6a}, R^{6b}, R^{6c}, and R^{6d}, when present, is independently selected from hydrogen, —F, and —Cl. In yet a further aspect, each of R^{6a}, R^{6b}, R^{6c}, and R^{6d}, when present, is independently selected from hydrogen and —Cl. In an even further aspect, each of R^{6a}, R^{6b}, R^{6c}, and R^{6d}, when present, is independently selected from hydrogen and —F.

In various aspects, each of R^{6a}, R^{6b}, R^{6c}, and R^{6d}, when present, is hydrogen.

Ar¹ Groups. In some aspects, Ar¹ is a structure having a formula selected from:

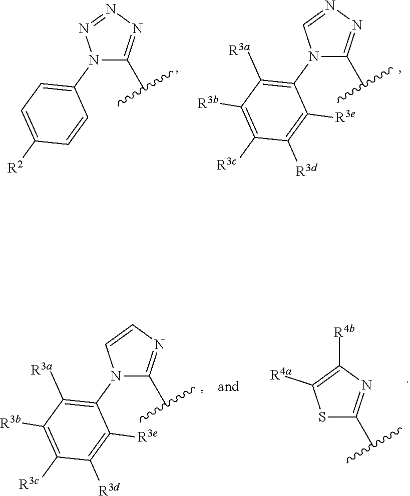

In some aspects, Ar¹ is a 5- to 10-membered heteroaryl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². Examples of 5- to 10-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, benzofuranyl, benzothiophenyl, pyridinyl, quinolinyl, and isoquinolinyl. In a further aspect, Ar¹ is a 5- to 10-membered heteroaryl substituted with 0, 1, or 2 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a still further aspect, Ar¹ is a 5- to 10-membered heteroaryl substituted with 0 or 1 substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In yet a further aspect, Ar¹ is a 5- to 10-membered heteroaryl monosubstituted with a substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In an even further aspect, Ar¹ is an unsubstituted 5- to 10-membered heteroaryl.

In various aspects, Ar is a tetrazolyl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, Ar¹ is a tetrazolyl substituted with 0, 1, or 2 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a still further aspect, Ar is a tetrazolyl substituted with 0 or 1 substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In yet a further aspect, Ar¹ is a tetrazolyl monosubstituted with a substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In an even further aspect, Ar¹ is an unsubstituted tetrazolyl.

In various aspects, Ar¹ is a triazolyl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, Ar¹ is a triazolyl substituted with 0, 1, or 2 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a still further aspect, Ar¹ is a triazolyl substituted with 0 or 1 substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In yet a further aspect, Ar¹ is a triazolyl monosubstituted with a substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In an even further aspect, Ar is an unsubstituted triazolyl.

In various aspects, Ar¹ is a imidazolyl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, Ar¹ is a imidazolyl substituted with 0, 1, or 2 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a still further aspect, Ar is a imidazolyl substituted with 0 or 1 substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In yet a further aspect, Ar¹ is a imidazolyl monosubstituted with a substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In an even further aspect, Ar¹ is an unsubstituted imidazolyl.

In various aspects, Ar¹ is a thiazolyl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a further aspect, Ar¹ is a thiazolyl substituted with 0, 1, or 2 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In a still further aspect, Ar is a thiazolyl substituted with 0 or 1 substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In yet a further aspect, Ar¹ is a thiazolyl monosubstituted with a substituent selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar². In an even further aspect, Ar¹ is an unsubstituted thiazolyl.

In a further aspect, Ar¹ is:

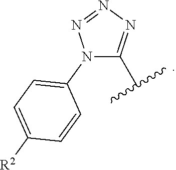

In a further aspect, Ar is selected from:

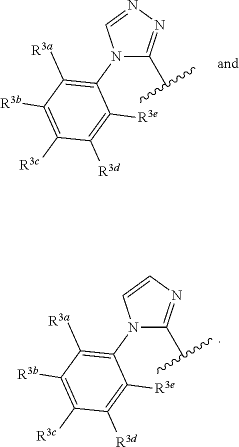

In a further aspect, Ar is:

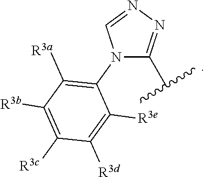

In a further aspect, Ar is:

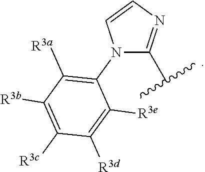

In a further aspect, Ar is:

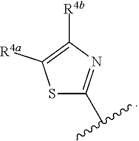

In a further aspect, Ar is selected from:

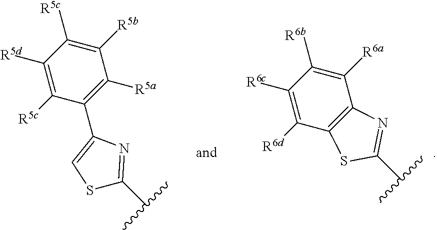

In a further aspect, Ar is selected from:

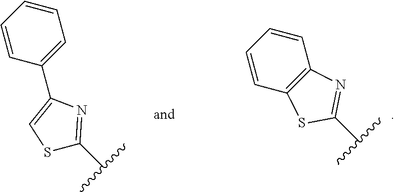 [SMILES: *c1nc(-c2ccccc2)cs1 ; *c1nc2ccccc2s1]

Ar² Groups

In some aspects, Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar², when present, is selected from C6 aryl and C3-C5 heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is selected from C6 aryl and C3-C5 heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is selected from C6 aryl and C3-C5 heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is selected from C6 aryl and C3-C5 heteroaryl, and is unsubstituted.

In various aspects, Ar², when present, is C6 aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, Ar², when present, is C6 aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is C6 aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is C6 aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is unsubstituted C6 aryl.

In various aspects, Ar², when present, is C3-C5 heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. Examples of C3-C5 heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, and pyridinyl. In a further aspect, Ar², when present, is C3-C5 heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a still further aspect, Ar², when present, is C3-C5 heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In yet a further aspect, Ar², when present, is C3-C5 heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In an even further aspect, Ar², when present, is unsubstituted C3-C5 heteroaryl.

Example Compounds. In some aspects, a compound can be present as one or more of the following structures:

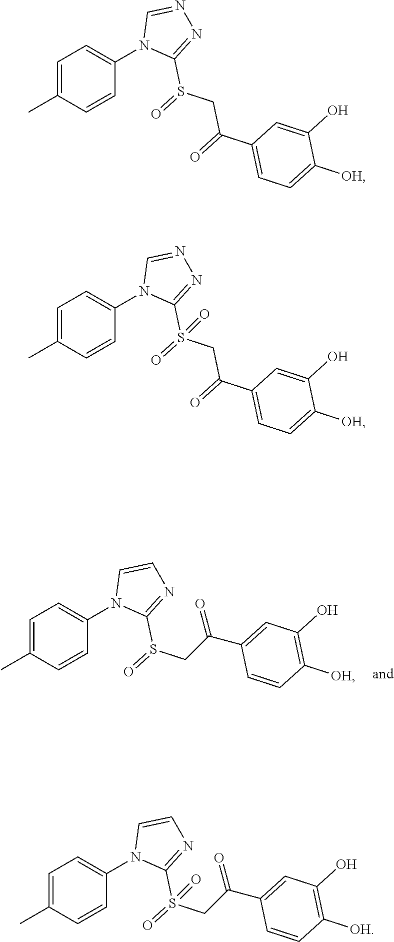 [SMILES: Cc1ccc(-n2cnnc2S(=O)CC(=O)c2ccc(O)c(O)c2)cc1 ; Cc1ccc(-n2cnnc2S(=O)(=O)CC(=O)c2ccc(O)c(O)c2)cc1 ; Cc1ccc(-n2ccnc2S(=O)CC(=O)c2ccc(O)c(O)c2)cc1 ; Cc1ccc(-n2ccnc2S(=O)(=O)CC(=O)c2ccc(O)c(O)c2)cc1]

or a pharmaceutically acceptable salt thereof.

In some aspects, a compound can be present as one or more of the following structures:

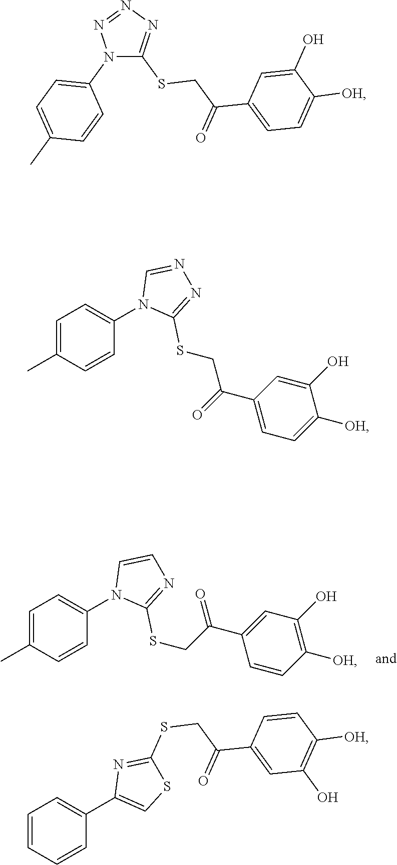

or a pharmaceutically acceptable salt thereof.

In some aspects, a compound can be present as one or more of the following structures:

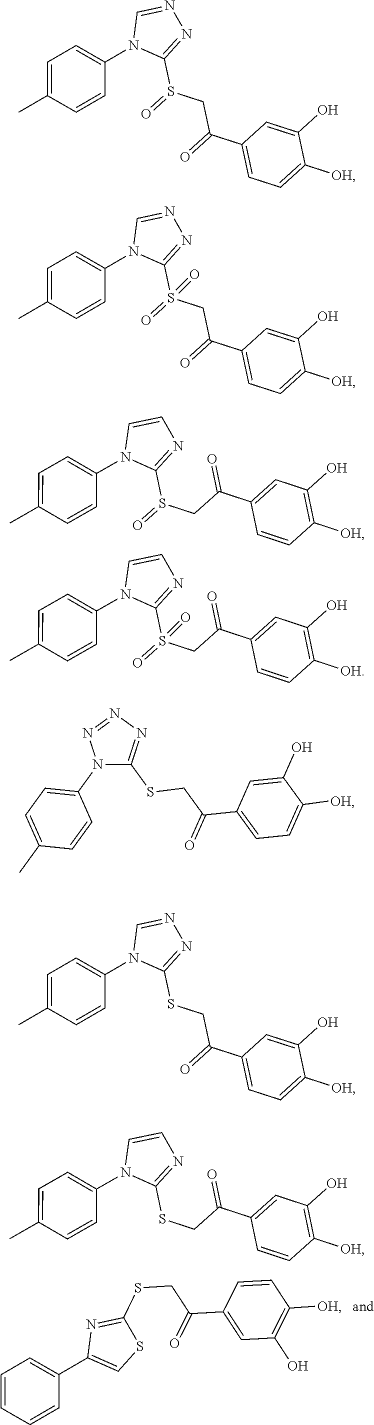

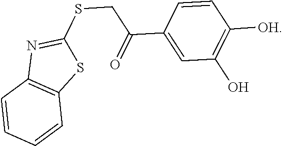 [SMILES: O=C(CSc1nc2ccccc2s1)c1ccc(O)c(O)c1]

or a pharmaceutically acceptable salt thereof.

Prophetic Thioethanone Examples. The following compound examples are prophetic, and can be prepared using the synthesis methods described herein above and other general methods as needed as would be known to one skilled in the art. It is anticipated that the prophetic compounds would be active as modulators of CARP-1 signaling, and such activity can be determined using the assay methods described herein below.

In some aspects, a compound can be selected from:

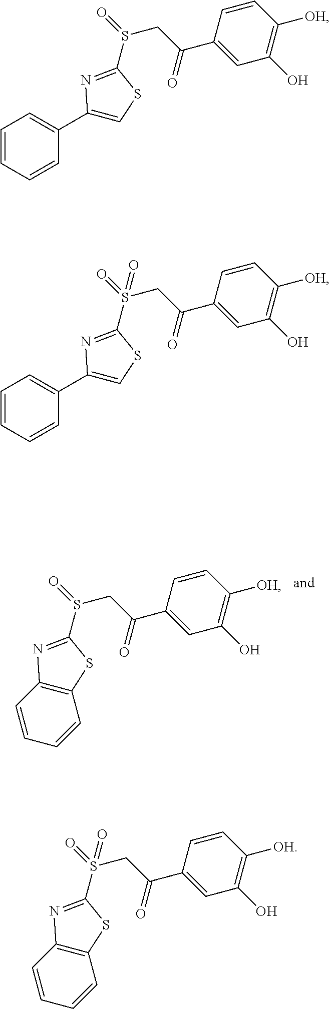 [SMILES: O=C(CS(=O)c1nc(-c2ccccc2)cs1)c1ccc(O)c(O)c1 ; O=C(CS(=O)(=O)c1nc(-c2ccccc2)cs1)c1ccc(O)c(O)c1 ; O=C(CS(=O)c1nc2ccccc2s1)c1ccc(O)c(O)c1 ; O=C(CS(=O)(=O)c1nc2ccccc2s1)c1ccc(O)c(O)c1]

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

Methods of Making a Compound.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VII, as described and exemplified below. The following examples are provided so that the invention might be more fully understood, are illustrative only, and should not be construed as limiting.

Route I. In some aspects, substituted tetrazole derivatives can be prepared as shown below.

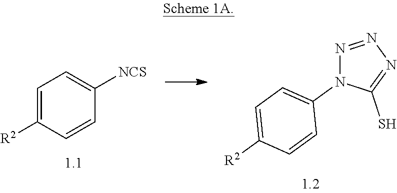

Compounds are represented in generic form, with substituents as noted in compound descriptions elsewhere herein. A more specific example is set forth below.

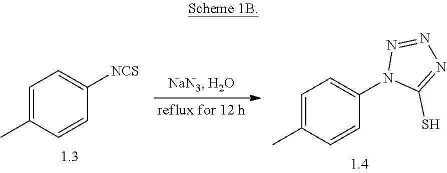

In some aspects, compounds of type 1.4, and similar compounds, can be prepared according to reaction Scheme 1B above. Thus, compounds of type 1.4 can be prepared by cyclization of an appropriate isothiocyanate, e.g., 1.3 as shown above. Appropriate isothiocyanates are commercially available or prepared by methods known to one skilled in the art. The cyclization is carried out in the presence of an appropriate azide, e.g., sodium azide, in an appropriate solvent, e.g., water, for an appropriate period of time, e.g., 12 hours under reflux conditions. As can be appreciated by one skilled in the art, the above reaction provides an example of a generalized approach wherein compounds similar in structure to the specific reactants above (compounds similar to compounds of type 1.1), can be substituted in the reaction to provide substituted tetrazole derivatives similar to Formula 1.2.

Route II. In some aspects, substituted triazole derivatives can be prepared as shown below.

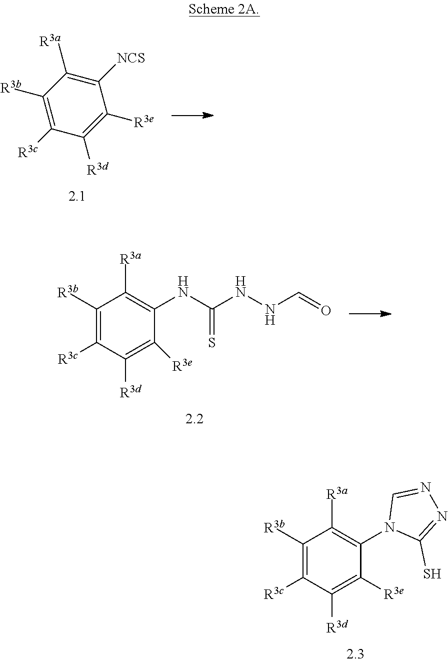

Compounds are represented in generic form, with substituents as noted in compound descriptions elsewhere herein. A more specific example is set forth below.

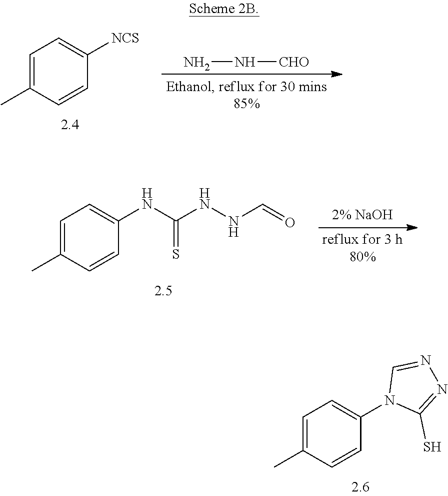

In some aspects, compounds of type 2.6, and similar compounds, can be prepared according to reaction Scheme 2B above. Thus, compounds of type 2.5 can be prepared by a coupling reaction between an appropriate isothiocyanate, e.g., 2.4 as shown above, and an appropriate hydrazide, e.g., formohydrazide as shown above. Appropriate isothiocyanates and appropriate formohydrazides are commercially available or prepared by methods known to one skilled in the art. The coupling reaction is carried out in the presence of an appropriate solvent, e.g., ethanol, for an appropriate period of time, e.g., 30 minutes under reflux conditions. Compounds of type 2.6 can be prepared by cyclization of an appropriate hydrazine carbothioamide, e.g., 2.5 as shown above. The cyclization is carried out in the presence of an appropriate base, e.g., 2% sodium hydroxide, for an appropriate period of time, e.g., 3 hours under reflux conditions. As can be appreciated by one skilled in the art, the above reaction provides an example of a generalized approach wherein compounds similar in structure to the specific reactants above (compounds similar to compounds of type 2.1 and 2.3), can be substituted in the reaction to provide substituted triazole derivatives similar to Formula 2.4.

Route III. In some aspects, substituted imidazole derivatives can be prepared as shown below.

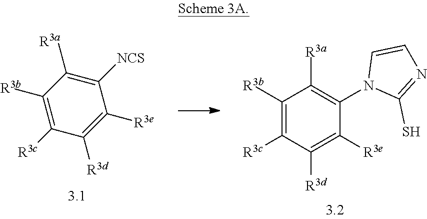

Compounds are represented in generic form, with substituents as noted in compound descriptions elsewhere herein. A more specific example is set forth below.

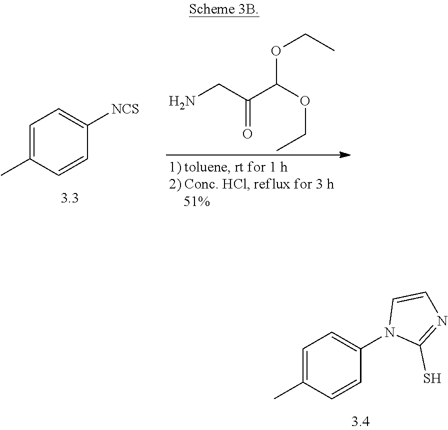

In some aspects, compounds of type 3.4, and similar compounds, can be prepared according to reaction Scheme 3B above. Thus, compounds of type 3.4 can be prepared by cyclization of an appropriate isothiocyanate, e.g., 3.3 as shown above. Appropriate isothiocyanates are commercially available or prepared by methods known to one skilled in the art. The cyclization is carried out in the presence of an appropriate amino-propanone, e.g., 3-amino-1,1-diethoxypropan-2-one, in an appropriate solvent, e.g., toluene, for an appropriate period of time, e.g., 1 hour under reflux conditions, followed by addition of an appropriate acid, e.g., concentrated hydrochloric acid, for an appropriate period of time, e.g., 3 hours under reflux conditions. As can be appreciated by one skilled in the art, the above reaction provides an example of a generalized approach wherein compounds similar in structure to the specific reactants above (compounds similar to compounds of type 3.1), can be substituted in the reaction to provide substituted imidazole derivatives similar to Formula 3.2.

Route IV. In some aspects, substituted thioethanone derivatives can be prepared as shown below.

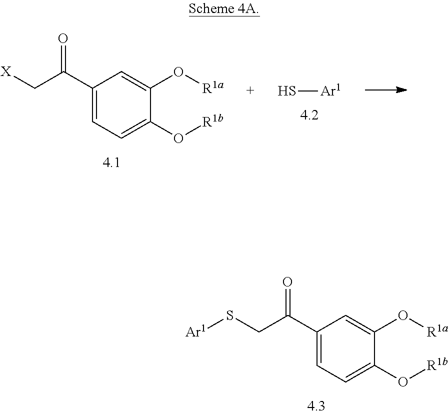

Compounds are represented in generic form, where X is a halogen, and with other substituents as noted in compound descriptions elsewhere herein. A more specific example is set forth below.

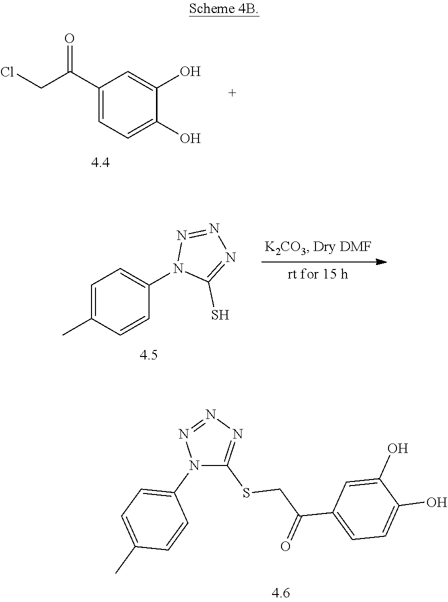 [SMILES: O=C(CCl)c1ccc(O)c(O)c1 ; Cc1ccc(-n2nnnc2S)cc1 ; Cc1ccc(-n2nnnc2SCC(=O)c2ccc(O)c(O)c2)cc1]

In some aspects, compounds of type 4.8, and similar compounds, can be prepared according to reaction Scheme 4B above. Thus, compounds of type 4.6 can be prepared by a coupling reaction between an appropriate halide, e.g., 4.4 as shown above, and an appropriate thiol, e.g., 4.5 as shown above. Appropriate halides and appropriate thiols are commercially available or prepared by methods known to one skilled in the art, or by methods disclosed herein. The coupling reaction is carried out in the presence of an appropriate base, e.g., potassium carbonate, in an appropriate solvent, e.g., dry dimethylformamide, for an appropriate period of time, e.g., 15 hours at room temperature. As can be appreciated by one skilled in the art, the above reaction provides an example of a generalized approach wherein compounds similar in structure to the specific reactants above (compounds similar to compounds of type 4.1 and 4.2), can be substituted in the reaction to provide substituted thioethanone derivatives similar to Formula 4.3.

Route V. In some aspects, substituted thioethanone derivatives can be prepared as shown below.

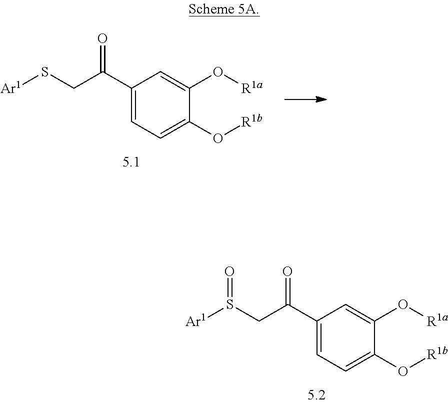

Compounds are represented in generic form, with substituents as noted in compound descriptions elsewhere herein. A more specific example is set forth below.

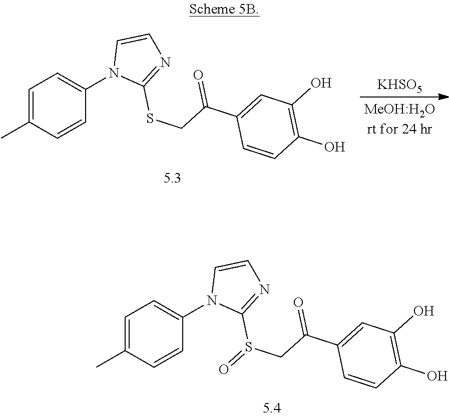

In one aspect, compounds of type 5.4, and similar compounds, can be prepared according to reaction Scheme 5B above. Thus, compounds of type 5.4 can be prepared by oxidation of an appropriate sulfide, e.g., 5.3 as shown above. Appropriate sulfides are prepared by methods known to one skilled in the art, or by methods disclosed herein. The oxidation is carried out in the presence of an appropriate oxidizing agent, e.g., potassium peroxymonosulfate, in an appropriate solvent, e.g., methanol: water, for an appropriate period of time, e.g., 24 hours at room temperature. As can be appreciated by one skilled in the art, the above reaction provides an example of a generalized approach wherein compounds similar in structure to the specific reactants above (compounds similar to compounds of type 5.1), can be substituted in the reaction to provide substituted thioethanone derivatives similar to Formula 5.2.

Route VI. In some aspects, substituted thioethanone derivatives can be prepared as shown below.

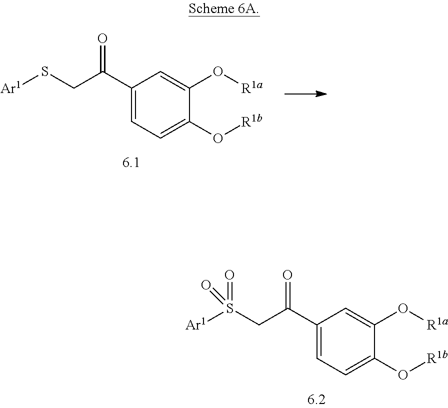

Compounds are represented in generic form, with substituents as noted in compound descriptions elsewhere herein. A more specific example is set forth below.

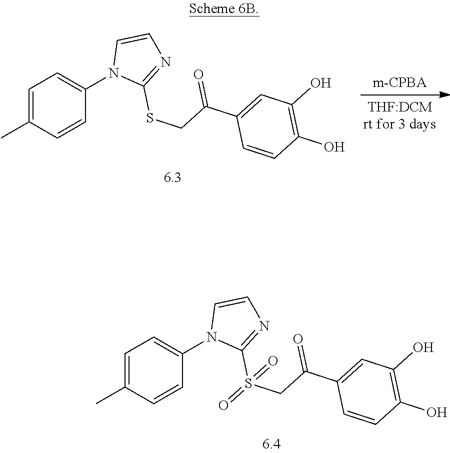 [SMILES: Cc1ccc(-n2ccnc2SCC(=O)c2ccc(O)c(O)c2)cc1 ; Cc1ccc(-n2ccnc2S(=O)(=O)CC(=O)c2ccc(O)c(O)c2)cc1]

In some aspects, compounds of type 6.4, and similar compounds, can be prepared according to reaction Scheme 6B above. Thus, compounds of type 6.4 can be prepared by oxidation of an appropriate sulfide, e.g., 6.3 as shown above. Appropriate sulfides are prepared by methods known to one skilled in the art, or by methods disclosed herein. The oxidation is carried out in the presence of an appropriate oxidizing agent, e.g., meta-chloroperoxybenzoic acid, in an appropriate solvent, e.g., tetrahydrofuran: dichloromethane, for an appropriate period of time, e.g., 3 days at room temperature. As can be appreciated by one skilled in the art, the above reaction provides an example of a generalized approach wherein compounds similar in structure to the specific reactants above (compounds similar to compounds of type 6.1), can be substituted in the reaction to provide substituted thioethanone derivatives similar to Formula 6.2.

Compositions

Disclosed herein are compositions for administering to a subject. Disclosed herein are compositions for treating a subject with a cancer. Disclosed herein are also compositions that can be useful for inhibiting cell cycle progression, cell growth or DNA repair. The compositions disclosed herein can also be useful for enhancing a chemotherapeutic response in a subject. Further, the compositions disclosed herein can be useful for reducing chemotherapeutic toxicity in a subject. The compositions disclosed herein can also be useful reducing or preventing chemotherapeutic resistance in a cancer cell. The compositions disclosed herein can be useful for inhibiting binding of NF-κB activating kinase IKK subunit γ (NEMO) to cell cycle and apoptosis regulatory protein (CARP)-1. The compositions disclosed herein can be useful for reducing systemic levels of one or more cytokines in a subject. Further, the compositions disclosed herein can be useful for enhancing the efficacy of radiotherapy and/or a chemotherapeutic agent.

Disclosed herein are compositions comprising a CARP-1-NEMO inhibitor and a DNA damage-inducing agent or a chemotherapeutic agent. In some aspects, the compositions can further comprise a pharmaceutical carrier. For example, disclosed herein are compositions comprising a CARP-1-NEMO inhibitor and a DNA damage-inducing agent or a chemotherapeutic agent, wherein the composition further comprise a pharmaceutical carrier. In some aspects, disclosed herein are compositions comprising a CARP-1-NEMO inhibitor and a DNA damage-inducing agent or a chemotherapeutic agent, wherein the CARP-1-NEMO inhibitor and the DNA damage-inducing agent or the chemotherapeutic agent are present in a therapeutically effective amount.

Disclosed herein are synergistic compositions for treating a subject with a cancer. In some aspects, the synergistic compositions comprise a cell cycle and apoptosis regulatory protein (CARP)-1—NF-κB activating kinase IKK subunit γ (NEMO) inhibitor, and a DNA damage-inducing agent or a chemotherapeutic agent. In some aspects, the synergistic compositions can further comprise a pharmaceutical carrier. In some aspects, the CARP-1-NEMO inhibitor and the DNA damage-inducing agent or the chemotherapeutic agent are present in a therapeutically effective amount.

In some aspects, the chemotherapeutic agent of the disclosed compositions is a DNA damage-inducing agent. In some aspects, the chemotherapeutic agent can be doxorubicin, cisplatin, 5-Fluorouracin (5-FU), etoposide, daunorubicin, camptothesin, methotrexate, carboplatin, or oxaliplatin. As used herein, a chemotherapeutic agent can also be a DNA damage-inducing agent that causes damage, for example, in the cellular DNA, by inducing single strand breaks or double strand breaks.

As used herein, a “DNA damage-inducing agent” or a “DNA damaging agent” refers to a composition or therapy that can modify the chemical structure of a nucleic acid. A “DNA damage-inducing agent” can also refer to a composition or therapy that can cause or create deletions or mutations in proteins associated with several DNA repair pathways that respond to damaged DNA. For example, a DNA damage-inducing agent can be a composition or therapy that causes DNA crosslinking, can prevent DNA synthesis (e.g. by inhibiting dihydrofolate reductase (DHFR), inhibiting topoisomerase II, or preventing or interfering with DNA replication). A DNA damage-inducing agent are widely used in oncology to treat both hematological and solid cancers. In some aspects, the DNA damage-inducing agent is a genotoxic stress-inducing agent. The DNA damage-inducing agent or genotoxic stress-inducing agent can be ultraviolet light, oxidative stress, chemical mutagens, or other compounds or therapies that lead to a variety of nucleotide modifications and DNA strand breaks such as ionizing radiation. In some aspects, the DNA damage-inducing agent can be doxorubicin, cisplatin, 5-Fluorouracin, etoposide, daunorubicin, camptothesin, methotrexate, carboplatin, oxaliplatin, or ionizing radiation.

In some aspects, the CARP-1-NEMO inhibitor can be 1-(3,4-dihydroxyphenyl)-2-{(1-(4-methylphenyl)-1H-tetrazol-5-yl)thio}ethanone (SNI-1). In some aspects, the CARP-1-NEMO inhibitor can be 2-{((4-methoxyphenyl)sulfonyl)amino}-N-(2-phenylethyl)benzamide (SNI-2). In some aspects, the CARP-1-NEMO inhibitor can be a SNI-1 analog. In some aspects, the SNI-1 analog can be any of the compounds in Table 2. In some aspects, any of the CARP-1-NEMO inhibitors disclosed herein can be in the form of a di-sodium salt.

TABLE 2
SNI-1 analogs.

| | |
|---|---|
| 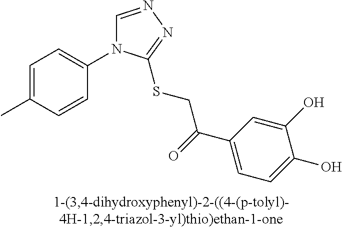 [SMILES: Cc1ccc(-n2cnnc2SCC(=O)c2ccc(O)c(O)c2)cc1] | GL-208 |
| 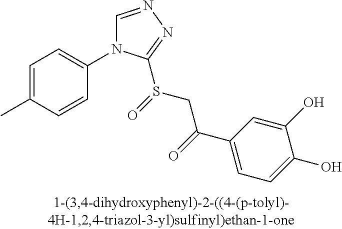 [SMILES: Cc1ccc(-n2cnnc2S(=O)CC(=O)c2ccc(O)c(O)c2)cc1] | GL-209 |
| 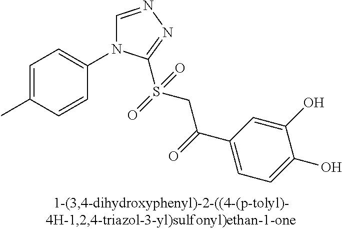 [SMILES: Cc1ccc(-n2cnnc2S(=O)(=O)CC(=O)c2ccc(O)c(O)c2)cc1] | GL-210 |
| 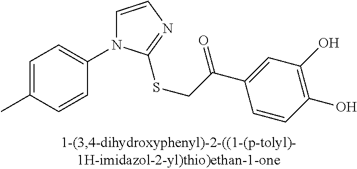 [SMILES: Cc1ccc(-n2ccnc2SCC(=O)c2ccc(O)c(O)c2)cc1] | GL-211 |
| 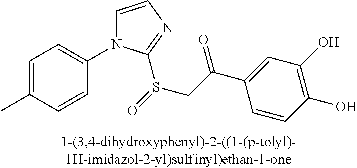 [SMILES: Cc1ccc(-n2ccnc2S(=O)CC(=O)c2ccc(O)c(O)c2)cc1] | GL-212 |
| 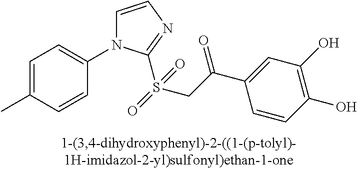 [SMILES: Cc1ccc(-n2ccnc2S(=O)(=O)CC(=O)c2ccc(O)c(O)c2)cc1] | GL-213 |
| 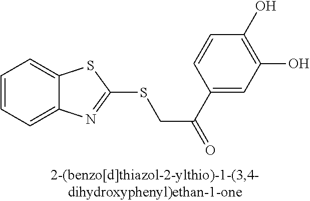 [SMILES: O=C(CSc1nc2ccccc2s1)c1ccc(O)c(O)c1] | GL-215 |
| 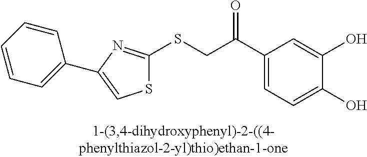 [SMILES: O=C(CSc1nc(-c2ccccc2)cs1)c1ccc(O)c(O)c1] | GL-216 |

Disclosed herein are compounds (e.g., CARP-1-NEMO inhibitors) having a structure represented by a formula:

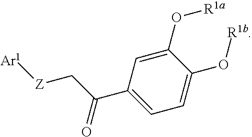

In some aspects, Z is selected from —S(O)— and —SO₂—. In some aspects, each of R^{1a} and R^{1b} is independently selected from hydrogen and C1-C4 alkyl.

In some aspects, each of R^{1a} and R^{1b} are covalently bonded, and, together with the intermediate atoms, comprise a 6-membered heterocycle. In some aspects, each of R^{1a} and R^{1b} together comprise —CH₂—. In some aspects, Art is a structure having a formula selected from:

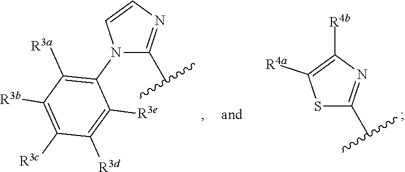

In some aspects, R², when present, is C1-C4 alkyl. In some aspects, each of R^{3a}, R^{3b}, R^{3c}, R^{3d}, and R³, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In some aspects, each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar², provided that at least one of R^{4a} and R^{4b}, when present, is not hydrogen. In some aspects, Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In some aspects, each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In some aspects, the compounds having a structure represented by a formula:

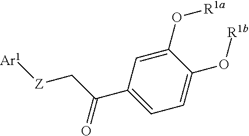

can be a pharmaceutically acceptable salt thereof.

In some aspects, Z is —S(O)—. In some aspects, wherein Z is —SO₂—. In some aspects, each of R^{1a} and R^{1b} is hydrogen. In some aspects, Ar¹ is:

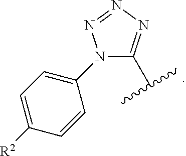

In some aspects, R² is methyl.

In some aspects, Ar¹ is selected from:

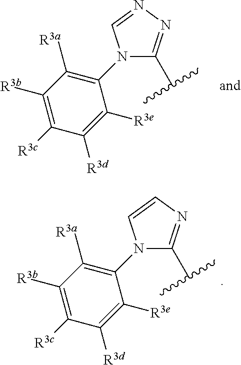

In some aspects, each of R^{3a}, R^{3b}, R^{3d}, and R^{3e} is hydrogen. R^{3c} is C1-C4 alkyl. In some aspects, R^{3e} is methyl.

In some aspects, Ar is:

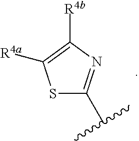

In some aspects, R^{4a} is hydrogen. In some aspects, R^{4b} is Ar². In some aspects, R^{4b} is unsubstituted phenyl.

In some aspects, the compound is selected from:

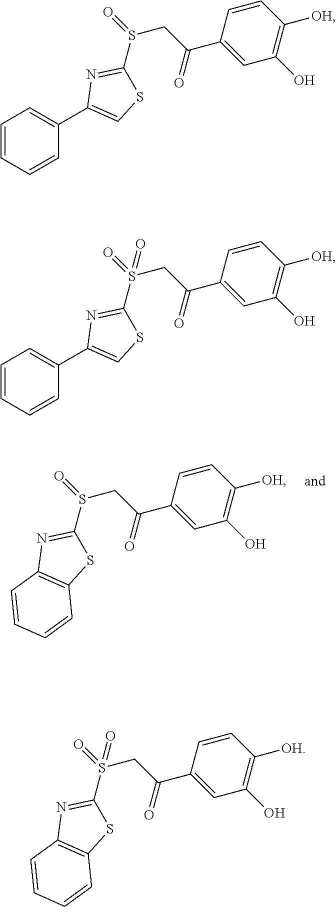 [SMILES: O=C(CS(=O)c1nc(-c2ccccc2)cs1)c1ccc(O)c(O)c1 ; O=C(CS(=O)(=O)c1nc(-c2ccccc2)cs1)c1ccc(O)c(O)c1 ; O=C(CS(=O)c1nc2ccccc2s1)c1ccc(O)c(O)c1 ; O=C(CS(=O)(=O)c1nc2ccccc2s1)c1ccc(O)c(O)c1]

In some aspects, the compound is selected from:

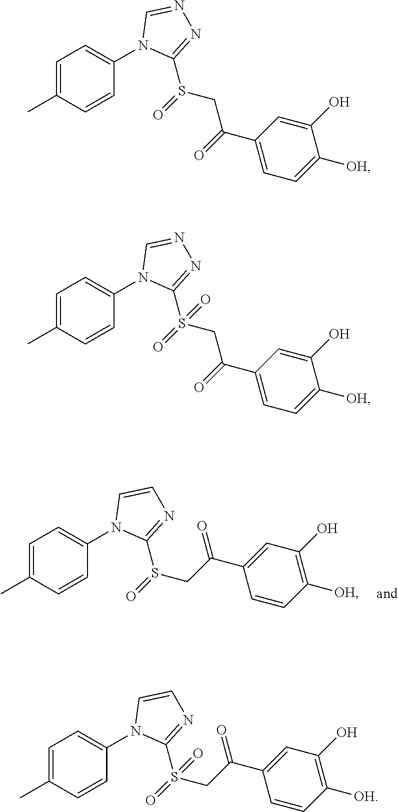 [SMILES: Cc1ccc(-n2cnnc2S(=O)CC(=O)c2ccc(O)c(O)c2)cc1 ; Cc1ccc(-n2cnnc2S(=O)(=O)CC(=O)c2ccc(O)c(O)c2)cc1 ; Cc1ccc(-n2ccnc2S(=O)CC(=O)c2ccc(O)c(O)c2)cc1 ; Cc1ccc(-n2ccnc2S(=O)(=O)CC(=O)c2ccc(O)c(O)c2)cc1]

Disclosed herein are compounds having a structure selected from:

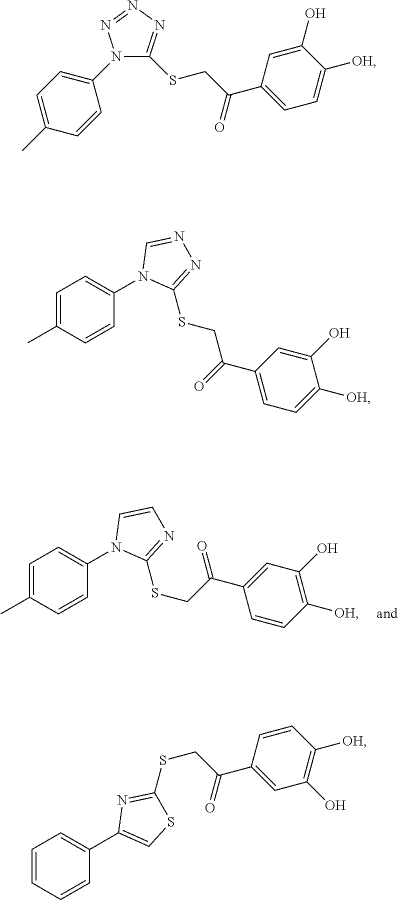 [SMILES: Cc1ccc(-n2nnnc2SCC(=O)c2ccc(O)c(O)c2)cc1 ; Cc1ccc(-n2cnnc2SCC(=O)c2ccc(O)c(O)c2)cc1 ; Cc1ccc(-n2ccnc2SCC(=O)c2ccc(O)c(O)c2)cc1 ; O=C(CSc1nc(-c2ccccc2)cs1)c1ccc(O)c(O)c1]

or a pharmaceutically acceptable salt thereof.

In some aspects, any of the compounds disclosed herein having a structure represented by a formula:

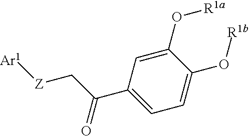

can be considered to be an SNI-1 analog.

In some aspects, any of the compounds disclosed herein having a structure represented by a formula:

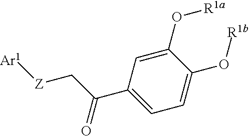

can be considered to be a CARP-1 NEMO inhibitor.

Pharmaceutical Compositions

As disclosed herein, are pharmaceutical compositions, comprising one or more of the compositions disclosed herein. In some aspects, the pharmaceutical compositions can comprise any of compositions disclosed herein. In some aspects, the pharmaceutical composition can comprise any of the compounds, CARP-1-NEMO inhibitors, chemotherapeutic agents, DNA damage-inducing agents disclosed herein or a combination thereof. For example, disclosed are pharmaceutical compositions, comprising a cell cycle and apoptosis regulatory protein (CARP)-1-NF-κB activating kinase IKK subunit γ (NEMO) inhibitor. Also, disclosed herein are pharmaceutical compositions comprising a CARP-1-NEMO inhibitor and one or more DNA damage-inducing agents or chemotherapeutic agents. In some aspects, the pharmaceutical composition can further comprise a pharmaceutically acceptable carrier.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the CARP-1-NEMO inhibitors, chemotherapeutic agents or DNA damage-inducing agents or combinations thereof described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, subcutaneous, intrathecal or intraperitoneal administration. Paternal administration can be in the form of a single bolus dose, or may be, for example, by a continuous pump. In some aspects, the compositions can be prepared for parenteral administration that includes dissolving or suspending the CARP-1-NEMO inhibitors in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The compositions disclosed herein can be formulated in a variety of combinations. The particular combination of the CARP-1-NEMO inhibitor with one or more chemotherapeutic agents (e.g., a DNA damage-inducing agent) can vary according to many factors, for example, the particular the type and severity of the cancer. The compositions described herein can be formulated to include a therapeutically effective amount of a CARP-I-NEMO inhibitor alone or in combination with one or more of the compounds disclosed herein (e.g., a DNA damage-inducing agent or a chemotherapeutic agent). In some aspects, a CARP-1-NEMO inhibitor can be contained within a pharmaceutical formulation. In some aspects, the pharmaceutical formulation can be a unit dosage formulation.

In some aspects, the CARP-1-NEMO inhibitor can be formulated for oral or parental administration. In some aspects, both the CARP-1-NEMO inhibitor and the chemotherapeutic agent or DNA damage-inducing agent can be formulated for oral or parental administration. In some aspects, the parental administration can be intravenous, subcutaneous, intramuscular or direct injection.

In some aspects, the compositions disclosed herein are formulated for oral, intramuscular, intravenous, or subcutaneous administration or direct injection.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be nucleic acids or vectors described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

The therapeutically effective amount or dosage of any of the CARP-1-NEMO inhibitors described herein, any of the chemotherapeutic agents, and any of the DNA damage-inducing agents used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

In some aspects, the therapeutically effective dose of any of the chemotherapeutic agents or any of the DNA damage-inducing agents described herein may be less/lower when combined with any of the CARP-1-NEMO inhibitors disclosed herein compared to the dose typically administered in the absence of a CARP-1-NEMO inhibitor. In some aspects, the administration of any of the CARP-1-NEMO inhibitors can increase the efficacy of any of the chemotherapeutic agents or any of the DNA damage-inducing agents described herein.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, any of the CARP-1-NEMO inhibitors disclosed herein alone or with any of the compounds disclosed herein can be administered in conjunction with standard therapies used to treat cancer (e.g., in combination with a DNA damage-inducing agent or the chemotherapeutic agent).

In some aspects, any of the CARP-1-NEMO inhibitors disclosed herein can be co-formulated with a DNA damage-inducing agent or the chemotherapeutic agent (e.g. doxorubicin, cisplatin, 5-Fluorouracin (5-FU), etoposide, daunorubicin, camptothesin, methotrexate, carboplatin, oxaliplatin, or ionizing radiation).

Any of the compounds or compositions described herein can be administered as a “combination.” It is to be understood that, for example, any of the CARP-1-NEMO inhibitors disclosed herein can be provided to the subject in need, either prior to administration DNA damage-inducing agent or a chemotherapeutic agent or any combination thereof, concomitant with administration of said DNA damage-inducing agent or chemotherapeutic agent or any combination thereof (co-administration) or shortly thereafter.

The dosage to be administered depends on many factors including, for example, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses. Dosage levels can be adjusted using standard empirical methods for optimization known by one skilled in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery.

Methods of Treatment

The composition and methods disclosed herein can be useful for the treatment of a subject with cancer. Disclosed herein are methods of treating cancer, the method comprising: administering to a subject with cancer a therapeutically effective amount of a cell cycle and apoptosis regulatory protein (CARP)-1—NF-κB activating kinase IKK subunit γ (NEMO) inhibitor. For example, disclosed herein are methods of treating cancer, the method comprising: administering to a subject with cancer a therapeutically effective amount of one or more of the compounds disclosed herein, 1-(3,4-dihydroxyphenyl)-2-{(1-(4-methylphenyl)-1H-tetrazol-5-yl)thio}ethanone (SNI-1), a SNI-1 analog or 2-{((4-methoxyphenyl)sulfonyl)amino}-N-(2-phenylethyl)benzamide (SNI-2). In some aspects, the therapeutically effective amount can reduce or diminish levels of DNA damage-induced pro-inflammatory cytokines. In some aspects, the therapeutically effective amount can enhance cancer growth suppression by DNA damage-inducing agents or chemotherapeutic agents (e.g., doxorubicin, daunorubicin, etoposide, camptothesin, methotrexate, 5-fluorouracil, and platinum compounds (e.g., cisplatin, carboplatin, and oxaliplatin). The method steps described herein can be carried out simultaneously or sequentially in any order.

The methods disclosed herein can be useful for inhibiting cell cycle progression, cell growth or DNA repair. Disclosed herein are methods of inhibiting cell cycle progression, cell growth or DNA repair, the methods comprises: contacting a cancer cell or malignant tissue with or administering to a subject with cancer a therapeutically effective amount of a CARP-1-NEMO inhibitor.

Disclosed herein are methods of enhancing a chemotherapeutic response in a subject, the methods comprise administering to a subject with cancer a therapeutically effective amount of a CARP-1-NEMO inhibitor.

Disclosed herein are methods of reducing chemotherapeutic toxicity in a subject, the methods comprise administering to a subject with cancer a therapeutically effective amount of a CARP-1-NEMO inhibitor and a therapeutically effective amount of chemotherapeutic agent or a DNA damage-inducing agent.

Disclosed herein are methods of reducing or preventing chemotherapeutic resistance in a cancer cell, the methods comprise administering to a subject with cancer a therapeutically effective amount of a CARP-1-NEMO inhibitor and a therapeutically effective amount of chemotherapeutic agent or a DNA damage-inducing agent.

Disclosed herein are methods of inhibiting binding of NEMO to CARP-1, the method comprising administering to a subject with cancer or contacting a cancer cell with a therapeutically effective amount of a CARP-1-NEMO inhibitor.

Disclosed herein are methods of reducing systemic levels of one or more cytokines in a subject, the methods comprise administering to a subject with cancer a therapeutically effective amount of a CARP-1-NEMO inhibitor.

Disclosed herein are methods of enhancing the efficacy of radiotherapy and/or a chemotherapeutic agent, the methods comprise: administering to a subject with cancer: (a) an effective amount of radiotherapy, the chemotherapeutic agent, DNA damage-inducing agent or a combination thereof; and (b) a therapeutically effective amount of a cell cycle and apoptosis regulatory protein (CARP)-1—NF-κB activating kinase IKK subunit γ (NEMO) inhibitor, wherein the administration of the CARP-1-NEMO inhibitor enhances the efficacy of the radiotherapy, the chemotherapeutic agent, DNA damage-inducing agent or a combination thereof in the subject with cancer.

In some aspects, the CARP-1-NEMO inhibitor can inhibit the binding of cell cycle and apoptosis regulatory protein (CARP)-1 to NF-κB activating kinase IKK subunit γ (NEMO). In some aspects, the CARP-1-NEMO inhibitor can decrease or suppress one or more pro-inflammatory cytokines. In some aspects, the one or more pro-inflammatory cytokines can be TNFα, IL-8, or IL-1β. In some aspects, the decrease or suppression of the one or more pro-inflammatory cytokines can reduce NF-κB activity.

In some aspects, the method can comprise administering to a subject with cancer a therapeutically effective amount of a CARP-1-NEMO inhibitor. In some aspects, the CARP-1-NEMO inhibitor can be a selective NF-κB inhibitor. In some aspects, the CARP-1-NEMO inhibitor can be one or more of the compounds disclosed herein, 1-(3,4-dihydroxyphenyl)-2-{(1-(4-methylphenyl)-1H-tetrazol-5-yl)thio}ethanone (SNI-1), a SNI-1 analog or 2-{((4-methoxyphenyl)sulfonyl)amino}-N-(2-phenylethyl)benzamide (SNI-2). In some aspects, the CARP-1-NEMO inhibitor can be administered with a pharmaceutically acceptable carrier. In some aspects, the CARP-1-NEMO inhibitor can be administered orally or parentally. In some aspects, the parental administration can be intravenous, intra-peritoneal, subcutaneous, intramuscular or direct injection.

Disclosed herein are methods of inhibiting cell growth and proliferation. In some aspects, the method can comprise contacting a cancer cell or malignant tissue with a therapeutically effective amount of a CARP-1-NEMO inhibitor. In some aspects, the method can comprise administering to a subject with cancer a therapeutically effective amount of a CARP-1-NEMO inhibitor. In some aspects, the cell cycle progression, the cell growth or the DNA repair can be inhibited directly or indirectly by reducing NF-κB activity.

Disclosed herein are methods of enhancing a chemotherapeutic response in a subject. In some aspects, the methods can comprise administering to a subject with cancer a therapeutically effective amount of a CARP-1-NEMO inhibitor. In some aspects, the CARP-1-NEMO inhibitor can enhance a chemotherapeutic response by increasing apoptosis. For example, administration CARP-1-NEMO inhibitor (e.g., SNI-1 or SNI-1 analog or one or more of the compounds disclosed herein) in combination with Adriamycin or cisplatin can increase the levels of the cleaved PARP or caspase 3 in cells when compared to administration of Adriamycin or cisplatin alone.

Disclosed herein are methods of enhancing a chemotherapeutic response in a subject. Disclosed herein are methods of enhancing a chemotherapeutic response in a subject, the methods comprising administering to a subject with cancer a therapeutically effective amount of a CARP-1-NEMO inhibitor.

Disclosed herein are methods reducing chemotherapeutic toxicity in a subject. Disclosed herein are methods reducing chemotherapeutic toxicity in a subject, the methods comprising administering to a subject with cancer a therapeutically effective amount of a CARP-1-NEMO inhibitor and a therapeutically effective amount of chemotherapeutic agent.

Disclosed herein are methods of reducing or preventing chemotherapeutic resistance in a cancer cell. Disclosed herein are methods of reducing or preventing chemotherapeutic resistance in a cancer cell, the methods comprising administering to a subject with cancer a therapeutically effective amount of a CARP-1-NEMO inhibitor and a therapeutically effective amount of a DNA damage-inducing agent or a chemotherapeutic agent.

Disclosed herein are methods of reducing systemic levels of one or more cytokines in a subject. Disclosed herein are methods of reducing systemic levels of one or more cytokines in a subject, the methods comprising administering to the subject with cancer a therapeutically effective amount of a CARP-1-NEMO inhibitor. In some aspects, the one or more cytokines can be TNFα, IL-8, or IL-1β.

Disclosed herein are methods of enhancing the efficacy of radiotherapy and/or a chemotherapeutic agent. Disclosed herein are methods of enhancing the efficacy of radiotherapy and/or a chemotherapeutic agent, the methods comprising administering to a subject with cancer an effective amount of radiotherapy and/or a chemotherapeutic agent; and a therapeutically effective amount of a CARP-1-NEMO inhibitor. In some aspects, the administration of the CARP-1-NEMO inhibitor can enhance the efficacy of the chemotherapeutic agent and/or the radiotherapy in the subject with cancer. In some aspects, the CARP-1-NEMO inhibitor can enhance a chemotherapeutic response by increasing apoptosis. For example, administration of a CARP-1-NEMO inhibitor (e.g., SNI-1 or SNI-1 analog or one or more of the compounds disclosed herein) in combination with Adriamycin or cisplatin can increase the levels of the cleaved PARP or caspase 3 in cells when compared to administration of Adriamycin or cisplatin alone.

Disclosed herein are methods of inhibiting binding of NEMO to CARP-1. Disclosed herein are methods of inhibiting binding of NEMO to CARP-1, the methods comprising administering to a subject with cancer or contacting a cancer cell with a therapeutically effective amount of a CARP-1-NEMO inhibitor.

In some aspects, the methods disclosed herein can comprise contacting a cell with a CARP-1-NEMO inhibitor. In some aspects, the CARP-1-NEMO inhibitor can reduce the system level or expression of one or more cytokines. In some aspects, the CARP-1-NEMO inhibitor can inhibit, interfere or suppress the binding of CARP-1 to NEMO. In some aspects, the CARP-1-NEMO inhibitor can bind to CARP-1. In some aspects, the CARP-1-NEMO inhibitor can bind to CARP-1 can bind to NEMO. In some aspects, the cell can be a mammalian cell. In some aspects, the mammalian cell can be a malignant cell. In some aspects, the malignant cell can be a brain cell, a breast cell, a kidney cell, a pancreatic cell, a lung cell, a colon cell, a prostate cell, a cell of the lymphatic system, a liver cell, an ovary cell, or a cervical cell.

In some aspects, the methods can further include the step of identifying a subject (e.g., a human patient) who has cancer and then providing to the subject a composition comprising a CARP-1-NEMO inhibitor as disclosed herein.

In some aspects, the subject has a cancer. In some aspects, the cancer can be a primary or a secondary tumor. In some aspects, the cancer can be a solid tumor. In some aspects, the cancer can be a non-solid tumor. In some aspects, the primary or secondary tumor can be within the subject's brain, breast, kidney, pancreas, lung, colon, prostate, lymphatic system, liver, ovary, or cervix. In some aspects, the primary or secondary tumor can be within the subject's bladder, stomach or thyroid. In some aspects, the cancer can be brain cancer, breast cancer, renal cancer, pancreatic cancer, lung cancer, liver cancer, lymphoma, prostate cancer, colon cancer, ovarian cancer, or cervical cancer. In some aspects, the cancer can be bladder cancer, stomach cancer, or thyroid cancer. In some aspects, the cancer can be a multiple myeloma or a soft tissue sarcoma. In some aspects, the cancer can be neuroblastoma or a medulloblastoma. In some aspects, the cancer can be a cancer that is treated with radiation or a DNA-damage-inducing agent. In some aspects, the cancer can be triple negative breast cancer. In some aspects, the cancer can be non-small cell lung cancer. In some aspects, the cancer can be diffuse large B cell lymphoma or follicular cell lymphoma.

The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of the condition or disease (e.g., cancer).

The compositions disclosed herein can be formulated in a variety of combinations. The particular combination of the CARP-1-NEMO inhibitor with one or more chemotherapeutic agents (e.g., a DNA damage-inducing agent) can vary according to many factors, for example, the particular the type and severity of the cancer. The compositions described herein can be formulated to include a therapeutically effective amount of a CARP-I-NEMO inhibitor alone or in combination with one or more of the compounds disclosed herein (e.g., a DNA damage-inducing agent or a chemotherapeutic agent). In some aspects, a CARP-1-NEMO inhibitor can be contained within a pharmaceutical formulation. In some aspects, the pharmaceutical formulation can be a unit dosage formulation.

In some aspects, the methods described herein can further comprise administering a therapeutically effective amount of a chemotherapeutic agent to the subject. The methods described herein can further comprise administering a therapeutically effective amount of a DNA damage-inducing agent to the subject. In some aspects, the chemotherapeutic agent can be a DNA damage-inducing agent. In some aspects, the chemotherapeutic agent can be doxorubicin, cisplatin, 5-Fluorouracin (5-FU), etoposide, daunorubicin, camptothesin, methotrexate, carboplatin, or oxaliplatin. In some aspects, the DNA damage-inducing agent can be ionizing radiation. In some aspects, the administration of a CARP-1-NEMO inhibitor can increase the efficacy of one or more of doxorubicin, cisplatin, 5-Fluorouracin (5-FU), etoposide, daunorubicin, camptothesin, methotrexate, carboplatin, or oxaliplatin. In some aspects, the administration of a CARP-1-NEMO inhibitor can increase the efficacy of ionizing radiation. In some aspects, a lower dose of any of the DNA damage-inducing agents can be administered when administered in combination with a CARP-1-NEMO inhibitor compared to the dose typically administered in the absence of the CARP-1-NEMO inhibitor. In some aspects, a lower dose of any of the chemotherapeutic agents can be administered when administered in combination with a CARP-1-NEMO inhibitor.

Methods of Screening

Disclosed herein are methods for screening one or more compounds for pharmacological intervention in cancer. In some aspects, the methods can comprise: (a) providing a CARP-1 amino acid fragment capable of binding to a NEMO amino acid fragment or a NEMO amino acid fragment capable of binding to a CARP-1 amino acid fragment. Examples of CARP-1 amino acids, CARP-1 amino acid fragments, NEMO amino acids, NEMO amino acid fragments and NEMO amino acid fragment capable of binding to a CARP-1 amino acid fragment can comprise one or more of the amino acid sequences provided in Table 1, or a fragment of one or more of the amino acid sequences provided in Table 1. In some aspects, the methods can further comprise providing a purified or non-purified compound or purified or non-purified mixture of compounds. In some aspects, the methods can further comprise screening the purified or non-purified compound or purified or non-purified mixture of compounds in an environment that allows for binding of the compound or mixture of compounds to the CARP-1 amino acid fragment or to the NEMO amino acid fragment. In some aspects, the methods can further comprise isolating the compound or mixture of compounds that are bound to either the CARP-1 amino acid fragment or the NEMO amino acid fragment. In some aspects, either the CARP-1 amino acid fragment or the NEMO amino acid fragment can be immobilized on a substrate. In some aspects, the binding of the one or more compounds to CARP-1 amino acid fragment or to the NEMO amino acid fragment can be measured by surface plasmon resonance.

In some aspects, the methods can further comprise determining the equilibrium dissociation constant of the one or more compounds to the CARP-1 amino acid fragment or the NEMO amino acid fragment. In some aspects, the CARP-1 amino acid fragment can be SEQ ID NO: SEQ ID NO: 6 (HRPEETHKGRTVPAHVETVVLFFPDVWHCL). In some aspects, the NEMO amino acid fragment can be SEQ ID NO: SEQ ID NO: 2 (EEKRKLAQLQVAYHQLFQEYDNHIKSSVVGSERKRGMQLE).

In some aspects, the CARP-1 amino acid fragment or the NEMO amino acid fragment can be conjugated to a detectable label or detection tag. Examples of detectable labels include but are not limited to floroscein for florescence, HA tag, Gst-tag, EGFP-tag, FLAG™ tag or biotin. In some aspects, the detectable label can be FLAG-tag or biotin. In some aspects, the label can be fused or conjugated to a CARP-1 amino acid fragment capable of binding to a NEMO amino acid fragment. In some aspects, the label can be fused or conjugated to a NEMO amino acid fragment capable of binding to a CARP-1 amino acid fragment.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody. The sequences of these tags are described in the literature and well known to the person of skill in art.

In some aspects, the disclosed methods and compositions comprise an epitope-tag wherein the epitope-tag has a length of between 6 to 15 amino acids. In an alternative aspect, the epitope-tag has a length of 9 to 11 amino acids.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

Kits

The kits described herein can include any combination of the compositions (e.g., CARP-1-NEMO inhibitors, chemotherapeutic agents and DNA damage-inducing agents) described herein and suitable instructions (e.g., written and/or provided as audio-, visual-, or audiovisual material). In some aspects, the kits can comprise a predetermined amount of a composition comprising any one of the compositions or combinations disclosed herein. The kit can further comprise one or more of the following: instructions, sterile fluid, syringes, a sterile container, delivery devices, and buffers or other control reagents.

### EXAMPLES

### Example 1: Antagonism of Cell Cycle and Apoptosis Regulatory Protein (CARP)-1 Binding with NEMO/IKKγ is a Novel Mechanism to Enhance Chemotherapy Efficacy

Abstract. NF-κB is a pro-inflammatory transcription factor that regulates immune responses and other distinct cellular pathways. Many NF-κB-mediated pathways for cell survival and apoptosis signaling by the transcription factor NF-κB are yet to be elucidated. CARP-1 is a perinuclear phospho-protein that regulates signaling by chemotherapy and growth factors. Although previous studies found CARP-1 to be a part of NF-κB proteome, regulation of NF-κB signaling by CARP-1, and the molecular mechanism(s) involved were not clarified. Disclosed herein are the findings that CARP-1 directly binds with NF-κB activating kinase IKK subunit γ (NEMO; NF-κB Essential Modulator), and regulates chemotherapy-activated canonical NF-κB pathway. Importantly, blockage of NEMO-CARP-1 binding diminishes NF-κB activation (noted by reduced phosphorylation of p65/RelA), indicated by reduced phosphorylation of its submit p65/RelA by chemotherapeutic Adriamycin (ADR) but not by NF-κB activation induced by TNFα, interleukin-1 (IL-1β) or EGF. High-throughput screening (HTS) of a chemical library yielded a small molecule inhibitor (SMI) of NEMO-CARP-1 binding, termed selective NF-κB inhibitor (SNI)-1. SNI-1 enhances chemotherapy-dependent growth inhibition of a variety of cancer cells including human triple-negative breast cancer (TNBC) cells, and patient-derived TNBC cells, in vitro, and attenuates secretion of chemotherapy-induced pro-inflammatory cytokines TNFα IL-1, and IL8. SNI-1 enhances Cisplatin inhibition of murine TNBC tumors, in vivo, and reduces systemic levels of pro-inflammatory cytokines. Thus, targeting and inhibiting NEMO-CARP-1 enhances responses of cancer cells to chemotherapy.

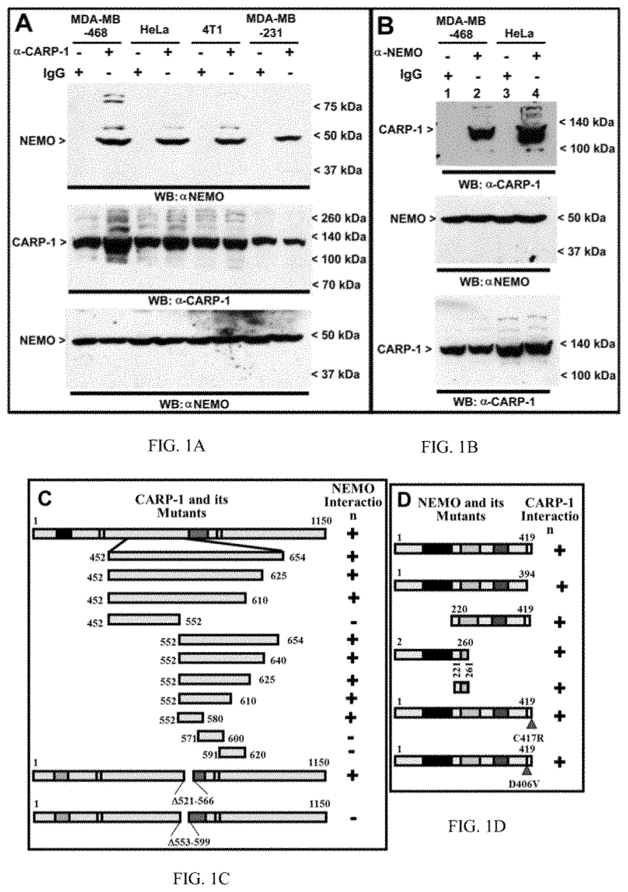

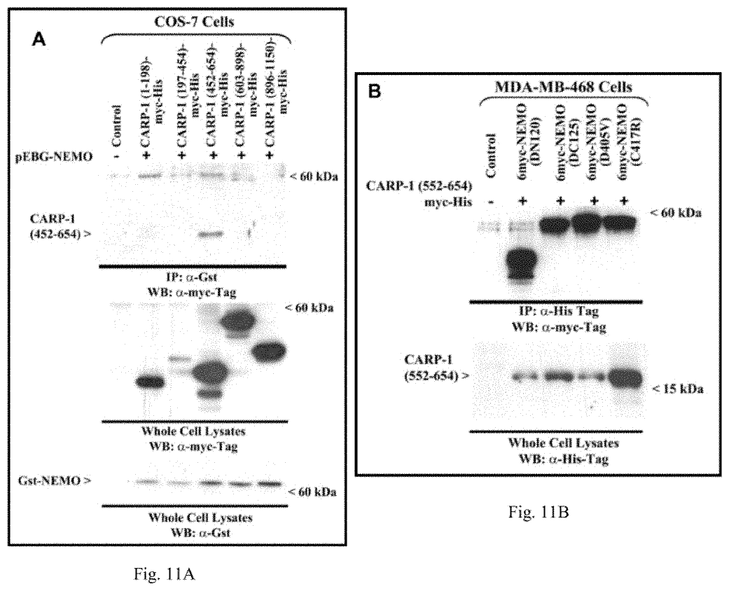

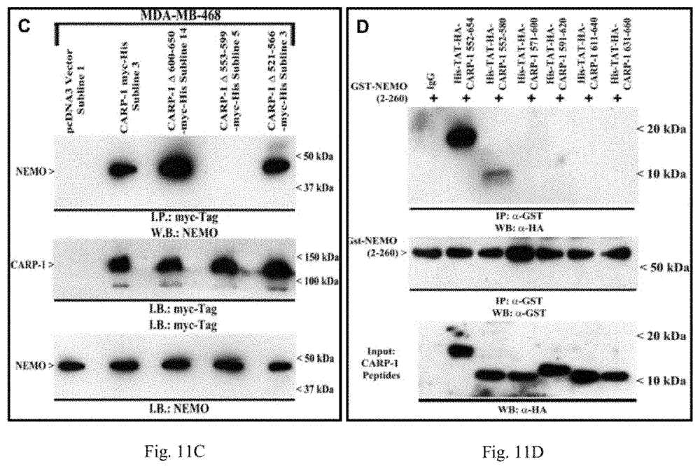

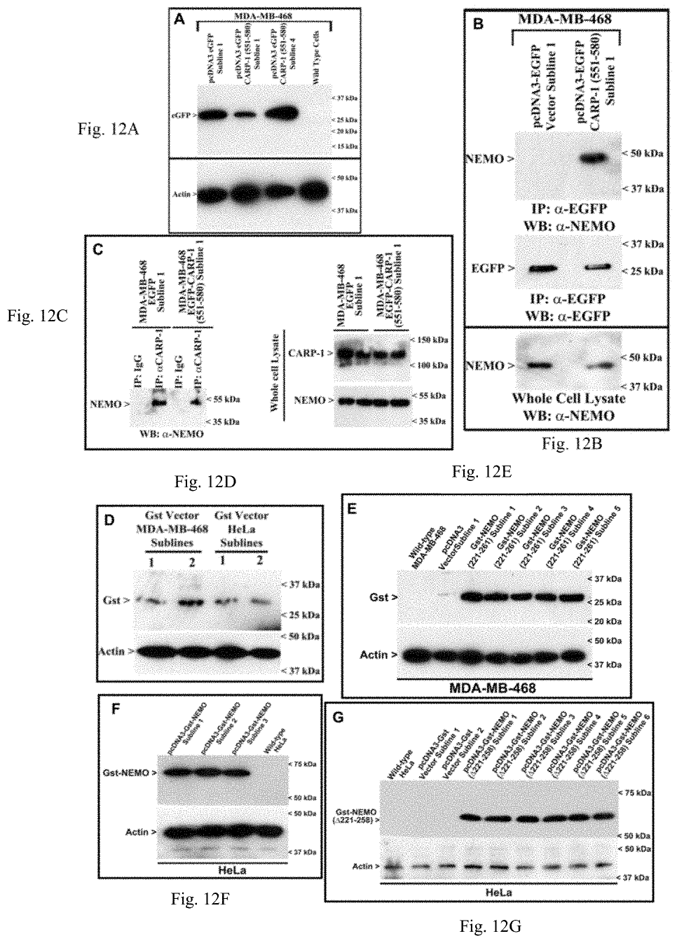

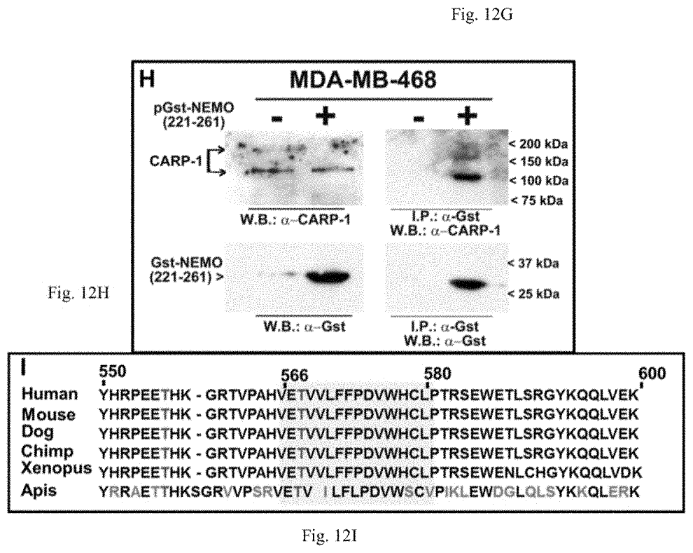

Results. CARP-1 binds with NEMO. It was previously found that TNFα, Adriamycin, or CARP-1 Functional Mimetic (CFM)-4 compound caused increased transcriptional activation of NF-κB in human TNBC cells while knock-down of CARP-1 attenuated activation of NF-κB by these agents (Muthu, M., et al. (2014) PLoS One 9, e102567). Since Adriamycin or epidermal growth factor receptor (EGFR) tyrosine kinase inhibitor Iressa inhibited HBC growth in part by inducing CARP-1 expression (Rishi, et al. (2003) J Biol Chem 278, 33422-33435; and Rishi, A. K., et al. (2006) J Biol Chem 281, 13188-13198), and CARP-1 was found to be a part of the NF-κB proteome (Bouwmeester, T., et al. (2004) Nat Cell Biol 6, 97-105), and CARP-1 was found to be a part of the NF-κB proteome, it was investigated whether and how CARP-1 regulates NF-κB signaling. Cellular proteins from the human and murine TNBC or human cervical cancer HeLa cells were immuno-precipitated using anti CARP-1 (α2) or NEMO antibodies followed by analysis of immuno-complexes by western blotting (WB) using NEMO or CARP-1 antibodies, respectively. The immuno-complexes derived from using anti-CARP-1 (α2) antibodies contained NEMO protein (FIG. 1A). As also shown in FIG. 1B, CARP-1 protein was present in the immuno-complexes derived from NEMO antibodies. These data in FIGS. 1A, B demonstrate that CARP-1 interacts with NEMO. Then, mutagenesis-based analyses were performed to map the interacting epitopes of CARP-1 and NEMO proteins. In the first instance, constructs expressing myc-His tagged, non-overlapping CARP-1 mutants were utilized (Rishi, A. K., et al. (2006) J Biol Chem 281, 13188-13198). Each of the CARP-1 mutant plasmids together with a plasmid expressing Gst-tagged NEMO (pEBG-NEMO) were separately transfected in COS-7 cells. Protein lysates were immuno-precipitated using anti-Gst antibodies followed by WB with anti-myc tag antibodies. NEMO interacted with CARP-1 (452-654; SEQ ID NO: 18) mutant (FIG. 11A). Next, HBC cells were transfected with various mutants of NEMO (32) together with a plasmid encoding myc-His-tagged CARP-1 (552-654; SEQ ID NO: 10) mutant. Protein lysates were immuno-precipitated using anti-His tag antibodies followed by WB with anti-myc tag antibodies. As shown in FIG. 11B, CARP-1 (552-654; SEQ ID NO: 10) mutant interacted with NEMO (221-405; SEQ ID NO: 19). Additional plasmids expressing myc-His-tagged CARP-1 proteins having in-frame deletions of amino acids 553-599 (SEQ ID NO: 4) or 521-566 (SEQ ID NO: 20) were generated and stable, neomycin-resistant HBC cells expressing these plasmids were obtained and characterized. Generation and characterization of HBC cells stably expressing pcDNA3 vector, myc-His tagged wild-type or CARP-1 (600-650; SEQ ID NO: 21) mutant were used (Rishi et al. (2006) J Biol Chem 281, 13188-13198; and Sekhar et al. (2019) Cancers (Basel) 11). Myc-tagged proteins were immuno-precipitated from stable sublines expressing vector, wild-type CARP-1, or CARP-1 mutant proteins followed by WB of immuno-complexes with NEMO antibodies. This experiment revealed CARP-1 amino acids 553-599 (SEQ ID NO: 4) harbored NEMO-binding epitope (FIG. 11C). Next, constructs for expression of Gst-NEMO (2-260; SEQ ID NO: 3) and His-TAT-HA tagged CARP-1 mutant peptides for expression in E. coli were generated. These E. coli expressed peptides were utilized to determine binding of NEMO (2-260; SEQ ID NO: 3) with various CARP-1 peptides. As shown in FIG. 11D, NEMO (2-260; SEQ ID NO: 3) bound with CARP-1 (552-654; SEQ ID NO: 10) and CARP-1 (552-580; SEQ ID NO: 22) peptides. The data in FIGS. 11A-D show that CARP-1 (552-580; SEQ ID NO: 22) and NEMO (221-260; SEQ ID NO: 7) harbor epitopes for their mutual interaction/binding. On this basis, pcDNA-based recombinant constructs were generated expressing EGFP, EGFP-CARP-1 (551-580; SEQ ID NO: 6), Gst, Gst-NEMO, Gst-NEMO (221-261; SEQ ID NO: 2), and Gst-NEMO (Δ221-258; SEQ ID NO: 23) proteins, and each construct was utilized to obtain stable, neomycin-resistant HBC or Hela sublines (FIGS. 12A, 12C-F). Immuno-precipitation and WB experiments further confirmed interaction of CARP-1 (551-580; SEQ ID NO: 6) with NEMO (FIG. 12B) and Gst-NEMO (221-261; SEQ ID NO: 2) with CARP-1 (FIG. 12G). FIG. 12 highlights conservation of the NEMO-interacting epitope of CARP-1 proteins deduced from various vertebrates and fly. Interactions of CARP-1 and NEMO and their respective mutants are summarized in FIGS. 1C and 1D. In sum, stable expression of CARP-1 (551-580; SEQ ID NO: 6) results in diminished interaction of endogenous NEMO with CARP-1 (FIG. 12C).

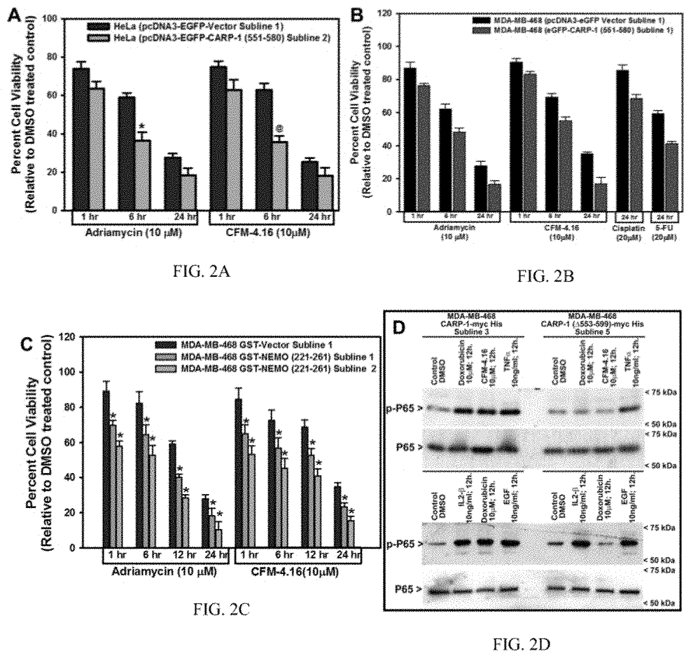

Interference of CARP-1 interaction with NEMO enhances Adriamycin efficacy in part through attenuation of RelA activation. To determine whether and to the extent CARP-1 interaction with NEMO regulated cell growth signaling, stable HBC and HeLa cells that express EGFP, EGFP-CARP-1 (551-580; SEQ ID NO: 6), Gst, Gst-NEMO (221-26; SEQ ID NO: 21), and CARP-1 (Δ553-599; SEQ ID NO: 4) proteins were utilized. Competition of endogenous CARP-1 binding with NEMO by overexpressing CARP-1 (551-580; SEQ ID NO: 6) or NEMO (221-261; SEQ ID NO: 2) resulted in a generally greater loss of cell viabilities following treatments with chemotherapeutics Adriamycin, Cisplatin, 5-Fluorouracil (5-FU), or an experimental compound CFM-4.16 when compared with the respective, vector expressing cells (FIGS. 2A-C). Next, it was clarified whether perturbation of CARP-1 binding with NEMO impacted NF-κB signaling. HBC cells that stably express myc-His tagged wild-type CARP-1 or CARP-1 (Δ553-599; SEQ ID NO: 4) mutant proteins were used. These cells were either untreated or separately treated with Adriamycin, CFM-4.16, TNFα, EGF, or IL1β followed by analysis of cell lysates by WB for expression of serine 536 phosphorylated or total RelA. As shown in FIG. 2D, the agents provoked a robust increase in RelA activation in cells expressing wild-type CARP-1. Serine 536 phosphorylation of p65, however, was diminished in cells expressing CARP-1 (Δ553-599) that were treated with Adriamycin or CFM-4.16, but not EGF, TNFα, or IL-1β (FIG. 2D). RelA activation, however, was diminished in cells expressing CARP-1 (Δ553-599; SEQ ID NO: 4) that were treated with Adriamycin or CFM-4.16, but not EGF, TNFα, or IL2β (FIG. 2D). These data show that NF-κB signaling involving p65 activation in the presence of Adriamycin or CFM-4.16 involves CARP-1 interaction with NEMO. Since Adriamycin and CFM-4.16 function in part by promoting DNA damage (Sekhar et al. (2019) Cancers (Basel) 11), and NEMO regulates activation of canonical NF-κB following DNA damage (Wu et al. (2006) Science 311, 1141-1146; and Huang (2003) Cell 115, 565-576), these findings would suggest for involvement of CARP-1 binding with NEMO for DNA damage-induced activation of canonical NF-κB pathway. DNA damage-induced signaling promotes NEMO sumoylation, its translocation to nucleus, followed by phosphorylation by the ATM/ATR kinase that results in NEMO mono-ubiquitination and nuclear export along with ATM to activate IKK kinase in cytosol (Wu et al. (2006) Science 311, 1141-1146; Huang (2003) Cell 115, 565-576; and Perkins, N. D. (2007) Nat Rev Mol Cell Biol 8, 49-62). Because CARP-1 is a perinuclear protein (Rishi et al. (2003) J Biol Chem 278, 33422-33435), it remains to be clarified whether CARP-1 interaction with NEMO regulates nuclear and/or cytoplasmic translocation of NEMO following DNA damage.

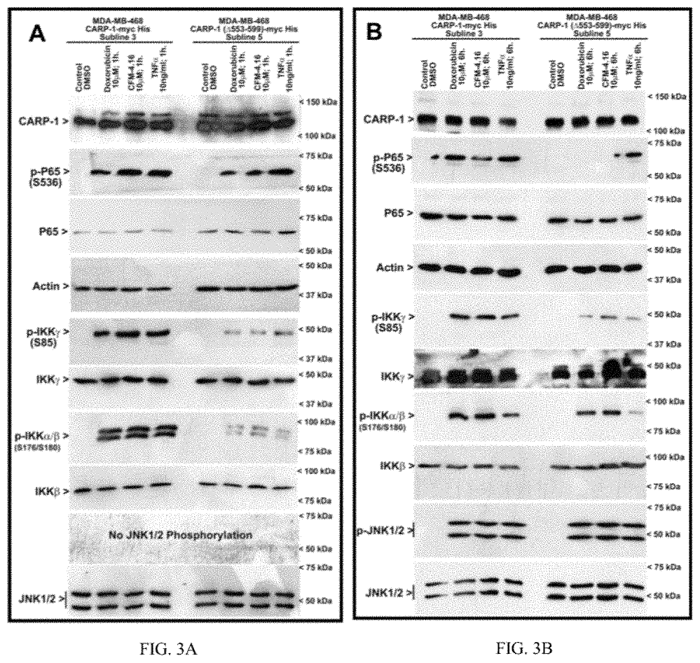

It was next investigated whether expression of CARP-1 (Δ551-599; SEQ ID NO: 24) mutant also interfered with activities of other important transducers of canonical NF-κB pathway. HBC cells stably expressing wild-type CARP-1 or CARP-1 (Δ551-599; SEQ ID NO: 24) mutant were separately treated with DMSO (Control), Adriamycin, CFM-4.16, or TNFα for a shorter (1 h) or longer (6 h) durations. WB analyses revealed a robust activation of p/65RelA, α/β and γ subunits of IKK occurred in cells expressing wild-type CARP-1 that were treated with Adriamycin, CFM-4.16, or TNFα over short (1 h) or long (6 h) durations (FIG. 3). Consistent with the data in FIG. 2D, activation of p65 was diminished in HBC cells expressing CARP-1 (Δ551-599; SEQ ID NO: 24) that were treated with Adriamycin or CFM-4.16 (FIG. 3B). Of note is that although a robust loss of p65 activation occurred in CFM-4.16 or Adriamycin-treated HBC cells expressing CARP-1 (Δ551-599; SEQ ID NO: 24) that were treated over longer (6 h) period, a moderate reduction in p65 activities also occurred in these cells that were treated over a shorter (1 h) period. Interestingly, expression of CARP-1 (Δ551-599; SEQ ID NO: 24) resulted in diminished serine 85 phosphorylation of IKKγ/NEMO regardless of the agent or duration of treatment, while activities of IKKα/were diminished in cells that were treated with respective agent for short (1 h) duration. However, a robust IKKα/β activation occurred in HBC cells expressing CARP-1 (Δ551-599; SEQ ID NO: 24) over a longer (6 h) treatments with CFM-4.16 or Adriamycin, but not TNFα. P65 activation was also observed in HBC cells expressing Gst-NEMO following treatments with IL-1β, EGF, and Adriamycin (FIG. 13A). Interference of CARP-1 binding with NEMO in the HBC cells with stable expression of NEMO (221-261; SEQ ID NO: 2) fragment resulted in attenuated p65 activation when treated with Adriamycin but not EGF or IL-1β (FIG. 13A). In addition, confocal microscopy-based in situ analysis revealed a reduction in serine 85 phosphorylation of IKKγ/NEMO in Adriamycin, CFM-4.16, or TNFα-treated HBC cells that express CARP-1 (Δ551-599; SEQ ID NO: 24) when compared with IKKγ/NEMO activation in Adriamycin, CFM-4.16, or TNFα-treated HBC cells that express wild-type CARP-1 (FIGS. 13A, 13B). Moreover, a 6 h but not 1 h, treatment with either of the agents provoked a robust activation of stress-activated mitogen-activated protein kinase (SAPK/MAPK) JNK1/2 in HBC cells expressing wild-type or Δ551-599 (SEQ ID NO: 24) mutant of CARP-1. These data collectively show that expression of CARP-1 (Δ551-599; SEQ ID NO: 24) interferes with serine 85 phosphorylation of IKKγ/NEMO in the presence of Adriamycin, CFM-4.16, or TNFα. Since serine 85 phosphorylation of NEMO by ATM kinase is required for NF-κB activation following DNA damage (Wu et al. (2006) Science 311, 1141-1146), and CARP-1 is a perinuclear protein (Rishi et al. (2003) J Biol Chem 278, 33422-33435), attenuation of NEMO phosphorylation at serine 85 in CFM-4.16 or Adriamycin-treated HBC cells that express CARP-1 (Δ551-599; SEQ ID NO: 24) would show that CARP-1 binding with NEMO is likely required for ATM-dependent phosphorylation of IKKγ/NEMO, and subsequent activation of IKK and RelA in cells treated with DNA damage inducing agents.

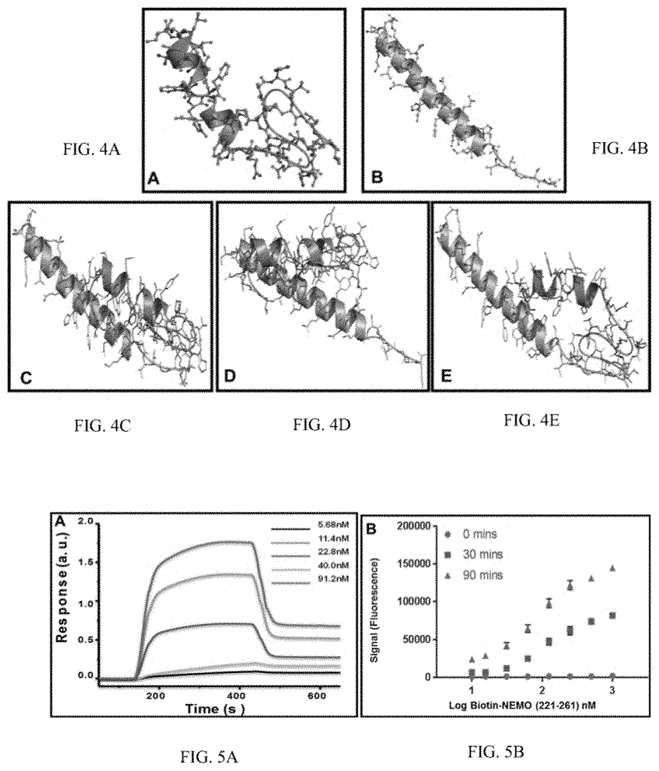

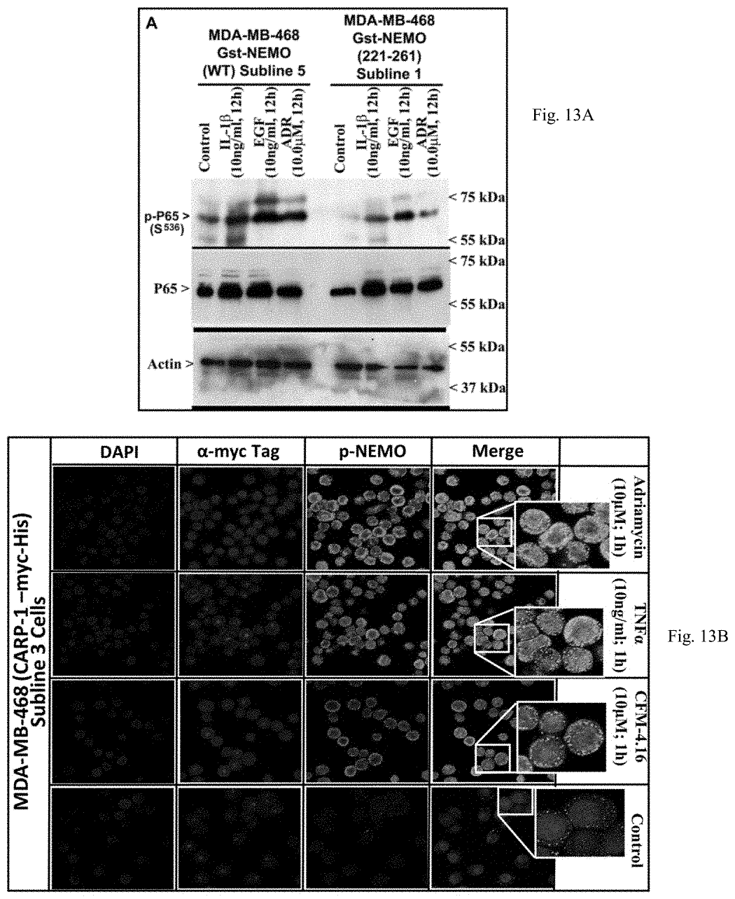

Kinetics of CARP-1 binding with NEMO, and identification of pharmacologic inhibitors of NEMO-CARP-1 interaction. Computational modeling and SPR studies were conducted to investigate the binding kinetics of CARP-1 (551-580; SEQ ID NO: 6) and NEMO (221-261; SEQ ID NO: 2) peptides (Sekhar et al. (2019) Cancers (Basel) 11). Since crystal structure of CARP-1 remains to be resolved, SWISS-MODEL (Waterhouse et al. (2018) Nucleic Acids Res 46, W296-w303) was used and indicated a 51.6% identity of CARP-1 (551-600; SEQ ID NO: 5) to TET2 resulting in a random coil domain (FIG. 4A). The crystal structure of NEMO is characterized and the NEMO (221-261; SEQ ID NO: 2) structure was obtained from the PDB (3CL3) (Bagneris et al. (2008) Mol Cell 30, 620-631) and is shown in FIG. 4B. Docking of these two models using ZDOCK 3.0.2 with IRaPPA re-ranking (Pierce et al. (2014) Bioinformatics 30, 1771-1773) permitted the top 3 predictions (FIGS. 4C-E) that were retained for further analysis via molecular dynamics (MD). Since small peptides have significantly more conformational freedom afforded compared to an entire protein, a larger fluctuation in the backbone root mean square deviation (RMSD) calculations was observed. Smaller values reflect greater stability of each complex throughout the simulations (FIG. 14). After solvation, equilibration, and heating, the structures undergo significant conformational change as expected to relieve clashes from docking. Complex 1 (FIG. 14A) shows a smooth increase throughout the 24 ns time course of the production run until an RMSD of roughly 10 Å after 7 ns. Beyond this point, the RMSD did not deviate significantly indicating a stable complex was reached. This was reflected in the histogram analysis by the Gaussian curve observed with a peak at an RMSD of 10 Å. No other dominant pose was observed. In complex 2 (FIG. 14B), there was significantly less shift in structure from the initial pose. The RMSD initially rose to approximately 8 Å but the structure relaxed to an area where the backbone RMSD leveled off at around 6 Å. Once again, the histogram analysis shows a Gaussian distribution with a peak at an RMSD of 6 Å for the highest occurrence. For complex 3 (FIG. 14C), an equilibrium was not reached as indicated by the continually rising backbone RMSD. Histogram analysis did not show any significantly dominant conformer, confirming no equilibrium was reached. Furthermore, binding energy calculations were conducted using MM-GBSA/PBSA to determine the potential of these two peptides to interact in a biological setting (Table 3). Calculations are taken from 200 snapshots sampled from the last 5 ns of simulation. The calculated binding energies for the three complexes was very similar since the difference between MM-GBSA and MM-PBSA values was not large. These data support the idea that CARP-1 (551-600; SEQ ID NO: 5) and NEMO (221-261; SEQ ID NO: 2) peptides are likely to form a relatively strong interaction in a biological setting.

TABLE 3
Calculation of binding energies (BE) (Kcal/M)
of the CARP-1 (551-600; SEQ ID NO: 5)/NEMO (221-
261; SEQ ID NO: 2) using MM-GBSA and MM-PBSA.

| | Complex 1 | | Complex 2 | | Complex 3 | |
|---|---|---|---|---|---|---|
| | B.E. | Std. | B.E. | Std. | B.E | Std. |
| | (kcal/mol) | Dev. | (kcal/mol) | Dev. | (kcal/mol) | Dev. |
| MM-GBSA | −53.7 | 5.9 | −66.1 | 5.9 | −44.3 | 8.2 |
| MM-PBSA | −59.0 | 6.5 | −75.5 | 8.1 | −55.1 | 9.6 |

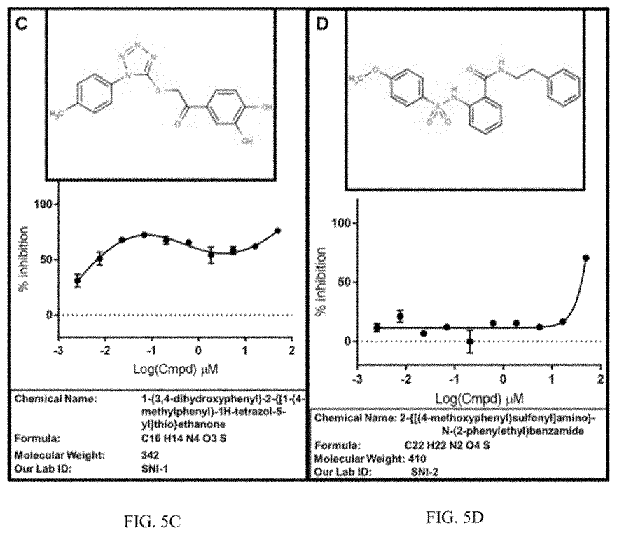

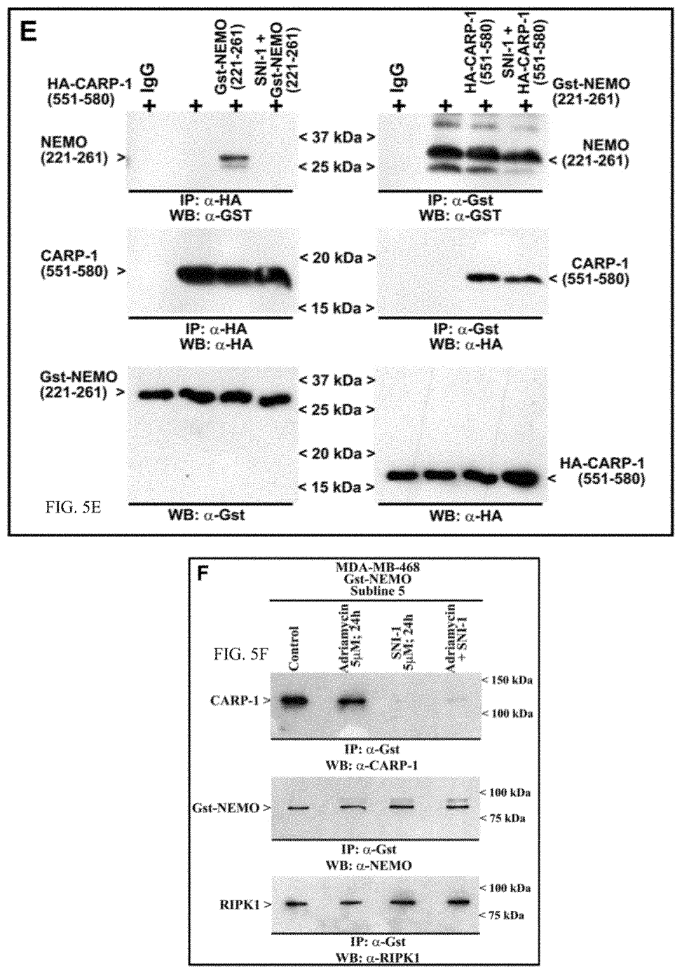

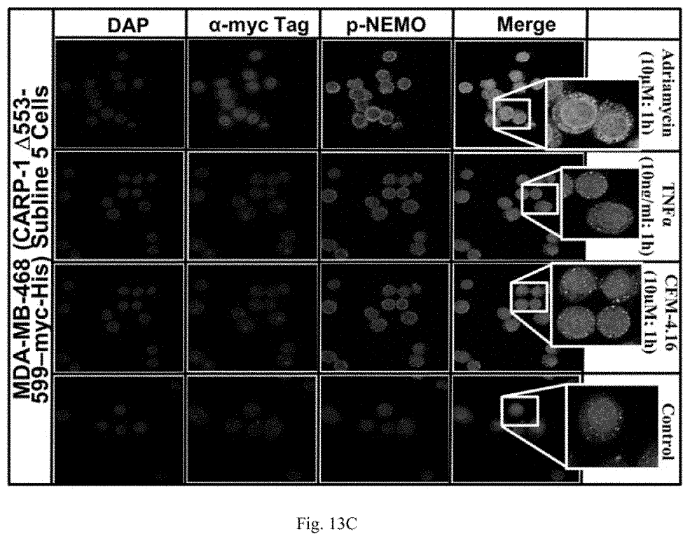

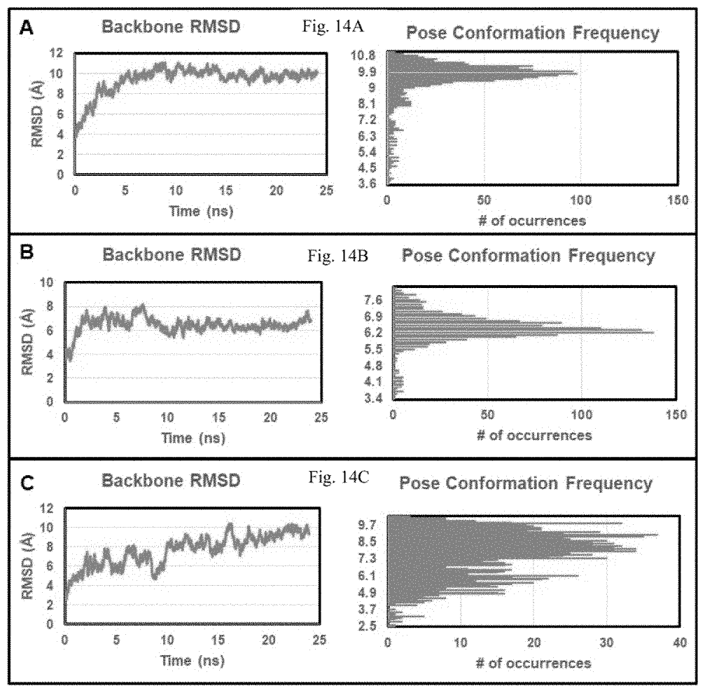

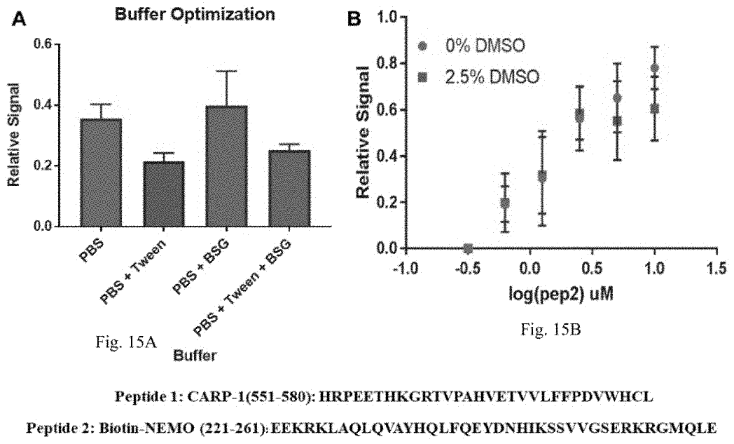

The predicted kinetics of interaction of CARP-1 (551-600; SEQ ID NO: 5) and NEMO (221-261; SEQ ID NO: 2) epitopes was further validated by utilizing respective, chemically synthesized peptides to determine their in-solution binding by SPR technology (Sekhar et al. (2019) Cancers (Basel) 11). As shown in FIG. 5A, this experiment revealed an equilibrium dissociation constant (K_{D} Value) of 1.02×10⁻⁷ M. (Ka=2.07×10³ M−1·s−1, Kd=2.12×10⁻⁴s⁻¹). On the collective basis of the data in FIG. 1, and biophysical and SPR data above, in vitro binding assays were developed utilizing chemically synthesized CARP-1 and NEMO peptides. In the first instance, CARP-1 (551-580; SEQ ID NO: 6) and NEMO (221-261; SEQ ID NO: 2) peptides were used to carry out buffer optimization and DMSO tolerance of the assay. The optimal binding was noted with PBS or PBS plus 0.01% bovine skin gelatin (BSG), and presence of 2.5% DMSO did not affect this binding (FIGS. 15A, 15B). Presence of 0.01% tween minimizes non-specific binding and generates higher reproducibility as noted by smaller error bars in FIG. 15A. For the purpose of high-throughput screening (HTS), the disclosed binding assay was adapted for use in ELISA based Alpha screen strategy (AlphaLISA; Perkin Elmer) by utilizing Flag-CARP-1 (546-580; SEQ ID NO: 8) and Biotin-NEMO (221-261; SEQ ID NO: 2) peptides. As shown in FIG. 5B, the assay demonstrated a robust interaction. The assay also demonstrated a Z′ factor >0.5 indicating suitable robustness threshold. HTS yielded two, small molecule inhibitors (SMI) of CARP-1 (546-580; SEQ ID NO: 8) binding with NEMO (221-261; SEQ ID NO: 2). Since interference of CARP-1 binding with NEMO resulted in attenuation of RelA activation and NF-κB signaling (FIG. 3), the compounds 1-(3,4-dihydroxyphenyl)-2-{(1-(4-methylphenyl)-1H-tetrazol-5-yl)thio}ethanone and 2-{[(4-methoxyphenyl)sulfonyl]amino}-N-(2-phenylethyl)benzamide (FIGS. 5C, D) were labelled as Selective NF-κB Inhibitors (SNI)-1 and -2, respectively. Interestingly, SNI-1 elicited a biphasic IC50 of ˜300 nM while the IC50 for SNI-2 was ˜25 μM in the AlphaLISA assay (FIGS. 5C, D). Although the precise reason for the biphasic IC50 for the SNI-1 compound is not known, of note is that the SNI-1 compound inhibited binding of CARP-1 (546-580; SEQ ID NO: 8) with NEMO (221-261; SEQ ID NO: 2) peptide with an IC50 that appears closer to the dissociation constant (K_{D}) of CARP-1 (551-580; SEQ ID NO: 6) and NEMO (221-260; SEQ ID NO: 7) peptides noted in the SPR assay (FIG. 5A). For this reason, the properties of SNI-1 compound was investigated further in biochemical and biological assays in vitro.

The biochemical mechanism of inhibition of CARP-1 (551-580; SEQ ID NO: 6) binding with NEMO (221-261; SEQ ID NO: 2) by SNI-1 was assessed. E. coli expressed Gst-NEMO (221-261; SEQ ID NO: 2) and His-TAT-HA-CARP-1 (551-580; SEQ ID NO: 6) peptides in IP-WB assays were used. As shown in FIG. 5E, incubation of SNI-1 with His-TAT-HA-CARP-1 (551-580; SEQ ID NO: 6) peptide that was immobilized with Ni-NTA beads abrogated binding of Gst-NEMO (221-261; SEQ ID NO: 2) with His-TAT-HA-CARP-1 (551-580; SEQ ID NO: 6) peptide. Incubation of SNI-1 with Gst-NEMO (221-261; SEQ ID NO: 2) peptide that was immobilized with Gst sepharose beads on the other hand failed to abrogate binding of His-TAT-HA-CARP-1 (551-580; SEQ ID NO: 6) with Gst-NEMO (221-261; SEQ ID NO: 2) peptide. These data show that SNI-1 binds with CARP-1 (551-580; SEQ ID NO: 6) epitope and prevents binding of NEMO (221-261; SEQ ID NO: 2) with CARP-1 (551-580; SEQ ID NO: 6). In light of the findings in FIG. 3 demonstrating CARP-1 involvement in ATM-dependent NEMO phosphorylation in the presence of Adriamycin, and since cytosolic ATM/NEMO/RIPK1 also regulates NF-κB response to DNA damage (Yang, Y., et al. (2011) Mol Cell Biol. 31, 2774-2786), it was determined whether CARP-1 was also involved in Adriamycin-induced NEMO/RIPK1 signaling. IP-WB analyses utilizing Gst-NEMO expressing HBC cells revealed that NEMO interacted with CARP-1 or RIPK1 in untreated, control and Adriamycin-treated cells (FIG. 5F). Presence of SNI-1 alone or in combination with Adriamycin abrogated NEMO interaction with CARP-1 but not with RIPK1 (FIG. 5F). Thus targeting of CARP-1 interaction with NEMO does not impact NEMO-RIPK-1 interaction. Since Adriamycin activates ATM to regulate canonical NF-κB and DSB repair pathways, it remains to be clarified whether CARP-1 regulates RIPK1 signaling dependent and/or independent of ATM. The data in FIG. 5 demonstrate that SNI-1 binds with CARP-1, and is a SMI of CARP-1 binding with NEMO.

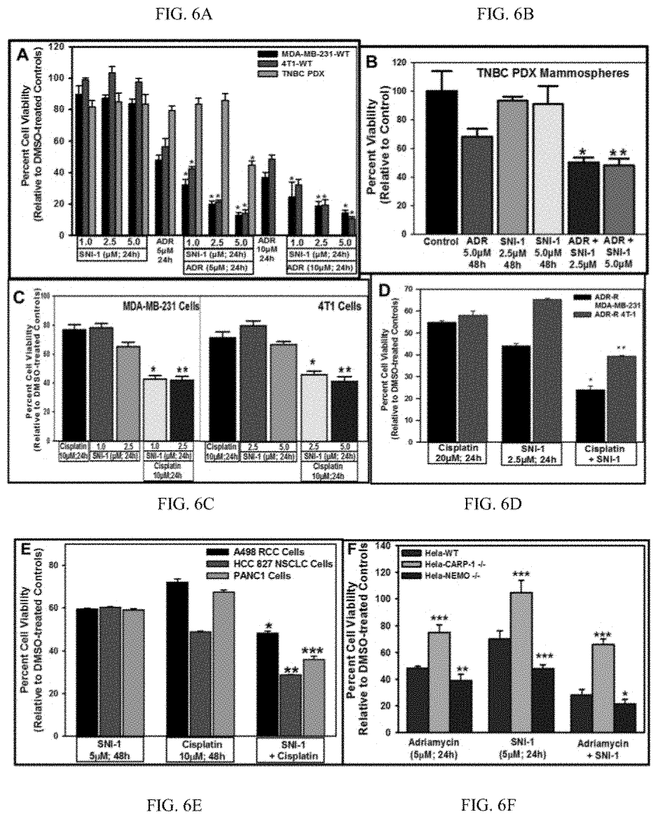

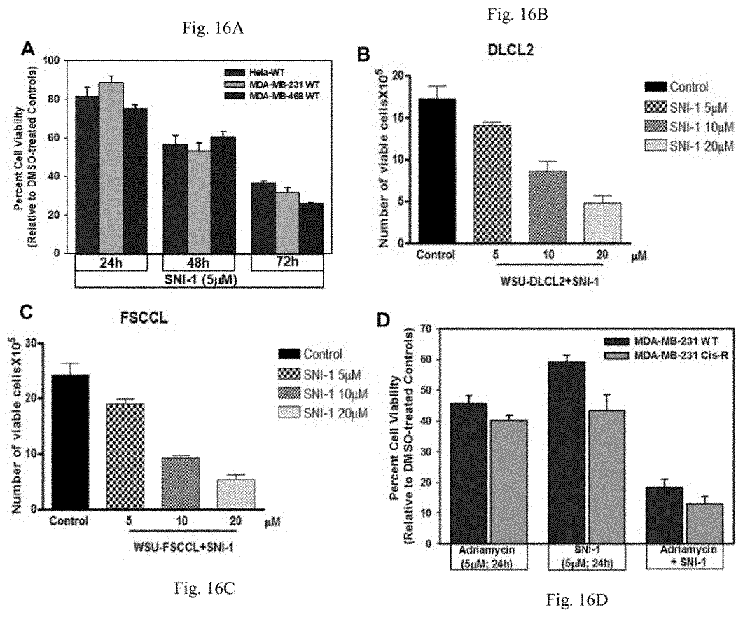

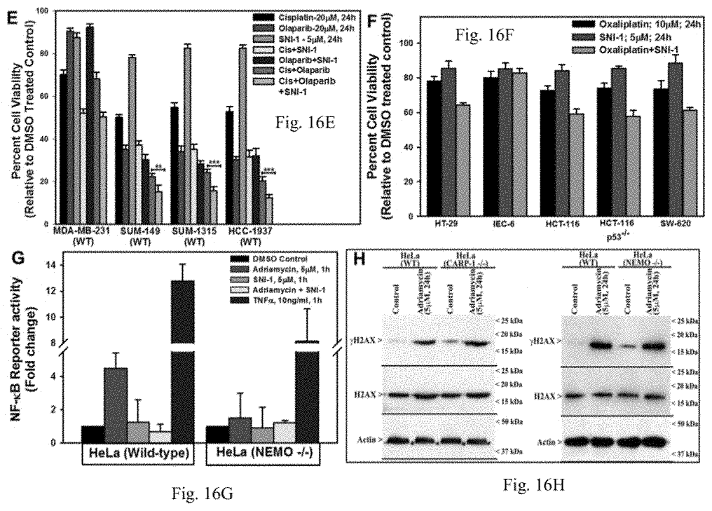

SNI-1 enhances efficacy of DNA damage-inducing chemotherapeutics, and inhibits secretion of pro-inflammatory and oncogenic cytokines by cancer cells in vitro and in vivo. The potential of SNI-1 compound as an inhibitor of cancer cell growth was investigated. Although SNI-1 doses of 1.0, 2.5, 5.0, or 10.0 μM over a 24 h period caused a modest, ˜10-20% loss of viability of the human MDA-MB-231 or the murine 4T1 TNBC cells, treatments of these TNBC cells with a 5.0 μM dose of SNI-1 over a 72 h period revealed an IC50 of ˜4.0-4.5 μM (FIG. 16A). SNI-1 treatments also resulted reduced viabilities of diffuse large B cell and follicular cell lymphoma cells with IC50s of ˜10.0 and 7.5 μM (FIGS. 16B, 16C). Given that Adriamycin inhibited growth of MDA-MB-231 TNBC cells with an IC50 of ˜3 μM (Wen et al. (2018) Cancer Cell Int 18, 128), human and murine TNBC cells as well as human PDX-derived TNBC cells were treated with a 5 μM dose of Adriamycin as a single agent or in combination with various doses of SNI-1 over a period of 24 h. Treatments of human and murine TNBC cells with a combination of Adriamycin and SNI-1 caused a significantly greater loss of cell viabilities when compared with cells treated with either agent alone (FIG. 6A). A statistically significant and greater loss of viability of human PDX-derived TNBC cells, however, also occurred in the presence of 5 μM each of SNI-1 and Adriamycin when compared with either compound alone (FIG. 6A). Although a 5 μM dose of Adriamycin for 48 h elicited ˜30% inhibition of mammospheres derived from human TNBC PDX tumors, a 2.5 or 5.0 μM dose of SNI-1 failed to inhibit growth of mammospheres derived from human PDX tumors (FIG. 6B). A statistically significant and greater loss of viability of mammospheres derived from human TNBC PDX tumors, however, occurred in the combined presence of SNI-1 and Adriamycin when compared with either compound alone (FIG. 6B). Adriamycin induces DSBs and activates NF-κB signaling that likely functions to promote DSB repair, survival, and eventual resistance of surviving cancer cells (Sekhar et al. (2019) Cancers (Basel) 11; Zhang et al. (2017) Cell 168, 37-57; Liu et al. (2006) Mol Cell 21, 467-480; Muthu et al. (2014) PLoS One 9, e102567). These data show that abrogation of NF-κB activation by SNI-1 likely interferes with DSB repair and cell survival with consequent increase in Adriamycin-induced viability loss of the TNBC cells. Chemotherapeutics such as 5-FU, Cisplatin as well as ionizing radiation also activate NF-κB signaling in various cancer cells (Wang et al. (2017) J Cancer Metastasis Treat 3, 45-59). Since Cisplatin forms covalent bonds with DNA resulting in intra-strand DNA adducts and crosslinks that in turn block transcription and replication (Rocha et al. (2018) Clinics (Sao Paulo) 73, e478s), it was tested whether SNI-1 also interferes with Cisplatin-dependent NF-κB signaling to enhance anti-cancer efficacy of Cisplatin. To test this possibility, parental and Adriamycin-resistant MDA-MB-231 and 4T1 TNBC cells (Cheriyan et al. (2016) Oncotarget 7, 73370-73388) were treated with Cisplatin, SNI-1, or a combination followed by measurement of cell viabilities as above. Treatments with a 10 μM dose of Cisplatin for 24 h caused a moderate 20-30% loss of viability of the TNBC cells, while a Cisplatin and SNI-1 combination elicited a marked, statistically significant loss of viabilities of these cells when compared with respective cells treated with either agent alone (FIG. 6C). Interestingly, treatments of Adriamycin-resistant TNBC cells with a combination of Cisplatin and SNI-1 also resulted in a greater loss of their viabilities when compared with respective cells that were treated with either agent alone (FIG. 6D). Similarly, a combination of Adriamycin and SNI-1 also provoked a greater loss of viability of the parental and Cisplatin-resistant TNBC cells when compared with cells that were treated with either agent (FIG. 16D). Since Cisplatin is a frontline clinical agent for treatment of non-small cell lung (NSCLC), renal, and pancreatic cancers, as well as a subset of BRCA-mutant TNBCs, these studies demonstrate a greater loss of viabilities of these cells when exposed to Cisplatin together with SNI-1 (FIG. 6E and FIG. 16E). Moreover, since Oxaliplatin is utilized for treatment of colon cancers, these studies also revealed a greater growth inhibition of different colon cancer cells that were treated with a combination of Oxaliplatin and SNI-1 when compared with cells that were treated with either agent alone (FIG. 16F). Consistent with the data disclosed herein with TNBC cells, treatments of human cervical cancer HeLa cells with a combination of Adriamycin and SNI-1 also provoked a greater loss of their viabilities when compared with SNI-1 or Adriamycin-treated cells (FIG. 6F). Crisper-based knock-out of CARP-1 was generated in HeLa cells (HeLa CARP-1 ko cells). In addition, HeLa NEOM ko cells (Fang et al. (2017) J Immunol 199, 3222-3233) were also used in the experiments described herein. Since interference of NEMO-CARP-1 interaction inhibited NF-κB signaling (FIG. 3), it was first clarified whether SNI-1 inhibited Adriamycin-induced transcriptional activation of NF-κB. For this purpose, wild-type and NEMO (ko) HeLa cells were used in conjunction with NF-κB-TATA-Luc reporter plasmid. FIG. 16G shows reduced NF-κB transcriptional activity in Adriamycin-treated NEMO (ko) cells when compared with their Adriamycin-treated, wild-type counterparts. Consistent with activation of NF-κB-mediated survival signaling by Adriamycin, treatments of the HeLa CARP-1 knock-out (ko) cells (HeLa CARP-1−/−) or HeLa NEMO ko cells (HeLa NEMO−/−) resulted in a significant increase or decrease, respectively, in their viabilities when compared with the viabilities of the Adriamycin-treated wild-type HeLa cells (FIG. 6F). Interestingly, although SNI-1 treatments resulted in a moderate, ˜30% reduction in the viabilities of wild-type HeLa cells, a further significant loss of viabilities of NEMO ko cells was noted following treatment with SNI-1 when compared with the similarly treated wild-type HeLa cells (FIG. 6F). Importantly, as also shown in FIG. 6F, SNI-1 failed to provoke any loss of viabilities of Hela CARP-1 ko cells suggesting a requirement of CARP-1 for transduction of signaling by SNI-1. In light of the fact that Adriamycin also activates ATM-dependent H2AX (γH2AX) to promote DSB repair signaling (Podhorecka, M., et al. (2010) J Nucleic Acids 2010), the WB analyses revealed a robust γH2AX levels in Adriamycin-treated HeLa cells regardless of the absence of CARP-1 or NEMO proteins (FIG. 16H). Thus, the data in FIG. 6 suggests that while CARP-1 regulates Adriamycin-induced canonical NF-κB signaling, CARP-1 is required for signaling by SNI-1. A combination of SNI-1 and the DNA damage inducing chemotherapeutics is a superior strategy for inhibiting growth of a variety of cancer cells including the drug-resistant TNBC cells.

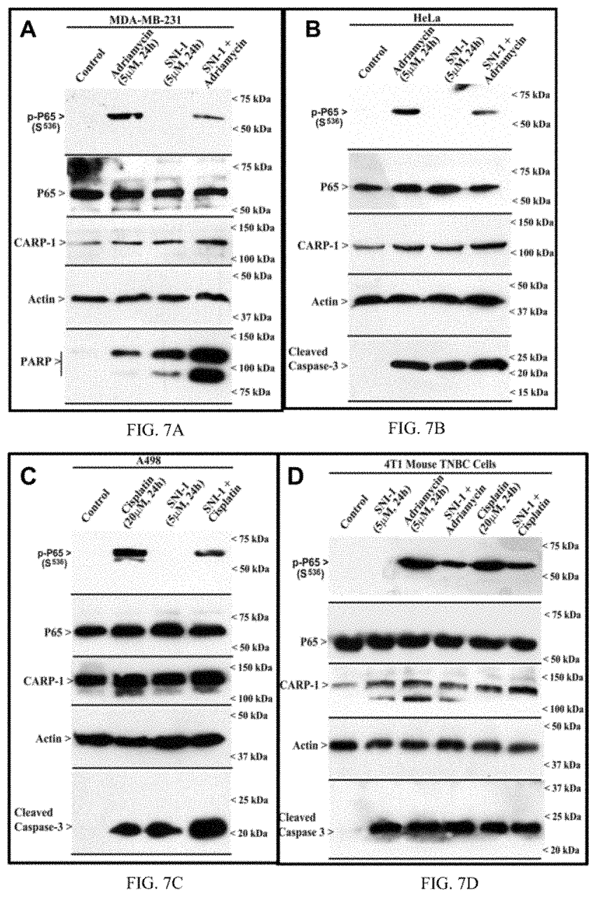

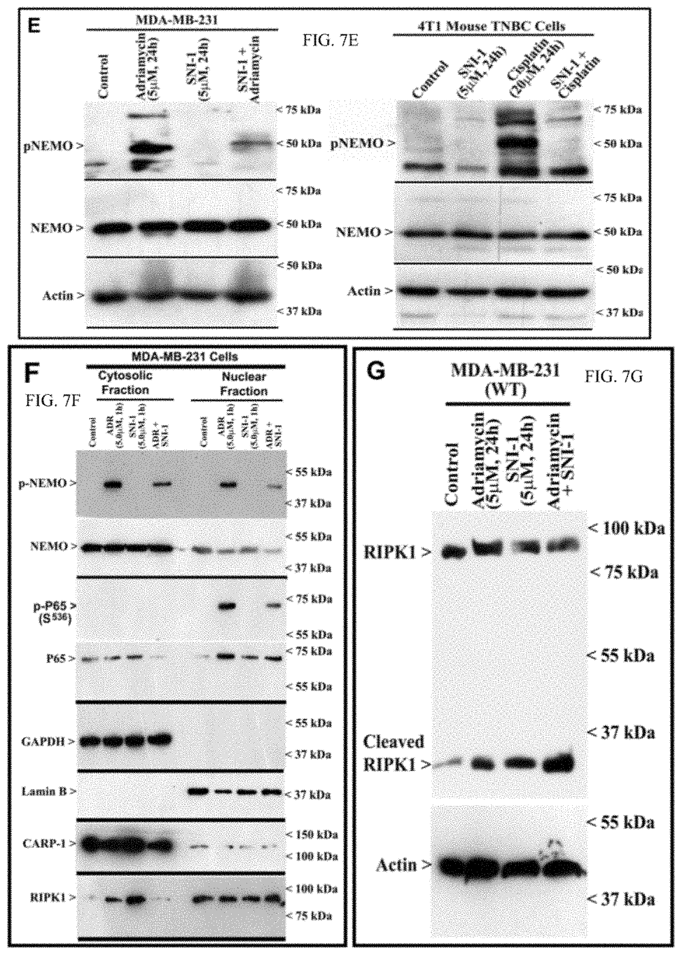

Since SNI-1 in combination with genotoxic chemotherapeutics provoked a greater loss of viability of a number of cancer cells, while SNI-1 alone also caused a moderate inhibition of cell growth, WB analysis revealed stimulation of apoptosis in cells exposed to SNI-1 as noted by elevated CARP-1 levels and cleavage of PARP or caspase-3 (FIGS. 7A-D). While treatments with Adriamycin or Cisplatin, but not SNI-1, also provoked a robust increase in p65/RelA activation and phosphorylation of NEMO, presence of SNI-1 generally resulted in diminished p65 activation and NEMO phosphorylation by Adriamycin or Cisplatin (FIG. 7A-E). Adriamycin, but not SNI-1, treatment caused p65 activation in nuclear compartment, while NEMO phosphorylation occurred in both the nuclear and cytosolic compartments (FIG. 7F). As expected, presence of SNI-1 interfered with Adriamycin-induced activation of p65 and NEMO phosphorylation, as well as resulted diminished cytosolic p65 levels (FIG. 7F). Since disruption of NEMO-CARP-1 interaction impacted Adriamycin-induced p65 activation (FIG. 3), and caspase-3 activation occurred in cells treated with Adriamycin, SNI-1, or a combination (FIG. 7A-D), it was next clarified whether NEMO was required for p65 and caspase activation by Adriamycin. The results show that Adriamycin provoked a robust p65 activation in HeLa wild-type, but not NEMO (ko), cells, while caspase-3 cleavage occurred in cells that were treated with Adriamycin, SNI-1, or a combination regardless of NEMO (FIG. 17). Moreover, since SNI-1 binds with CARP-1 (FIG. 5E), and CARP-1 interacts with RIPK1 (Muthu, M., et al. (2015) J. Biomed. Nanotechnol. 11: 1608-1627), and although SNI-1 did not interfere with RIPK1 interaction with NEMO (FIG. 5F), it was next clarified whether SNI-1 also regulated RIPK1 signaling. The WB analysis revealed that ADR or SNI-1 induced expression of cleaved RIPK1 (FIG. 7G). Interestingly, a combination of ADR and SNI-1 provoked a robust increase in cleaved RIPK1 (FIG. 7G). Cleavage of RIPK1 and caspase 3 will be consistent with prior studies demonstrating apoptosis signaling by DSB-inducing genotoxic chemotherapeutics that promote RIPK1 cleavage and activation of pro-apoptotic caspases-3, -6, and -8 (vanRaam, B. J., et al. (2013) Cell Death Differen. 20, 86-96). These findings show that blockage of CARP-1 binding with NEMO interferes with chemotherapy-activated NEMO phosphorylation and p65RelA activation to attenuate canonical NF-κB signaling.

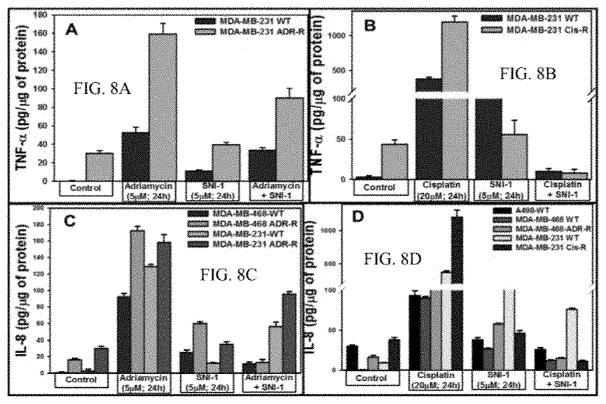

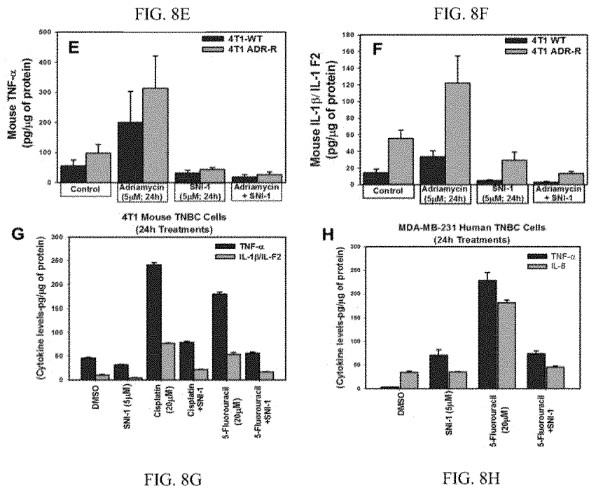

RelA regulates transcriptional activation of NF-κB target genes including a number of pro-inflammatory cytokines through the canonical and atypical pathways (Perkins, N. D. (2007) Nat Rev Mol Cell Biol 8, 49-62). Moreover, the DNA damage-inducing chemotherapeutics such as Adriamycin, Cisplatin, or 5-FU induce inflammatory cytokines that function in part to promote survival and resistance of cancer cells (Vyas et al. (2014) Onco Targets Ther 7, 1015-1023). Next, it was determined whether and to the extent presence of SNI-1 would attenuate chemotherapy-induced secretion of pro-inflammatory cytokines by cancer cells. Treatments with SNI-1 provoked a modest increase in levels of pro-inflammatory cytokines TNFα and IL-8 in culture media of human TNBC cells when compared with the levels of these cytokines in the media from respective, untreated cells (FIGS. 8A-D, H). SNI-1 treatments, however, failed to cause increase in secretion of TNFα and IL-1β in murine TNBC cells (FIG. 8E-G). As expected, treatments of parental and chemo-resistant human and murine TNBC cells and the parental renal cancer cells with Adriamycin, 5-FU, or Cisplatin provoked a robust increase in levels of TNFα, IL-8, and IL-1β in the media of the respective cell line. Consistent with attenuation of p65/RelA activation in cells that were exposed to a combination of SNI-1 and Adriamycin or Cisplatin, presence of SNI-1 also caused a robust decline in secretion of chemotherapy-induced pro-inflammatory cytokines TNFα, IL-8, and IL-1β (FIG. 8A-H). The data in FIG. 7 and FIG. 8 collectively show that pharmacologic blockage of CARP-1-NEMO binding functions to enhance chemotherapy efficacy in part by promoting superior growth inhibition of cancer cells, and reducing activation of canonical NF-κB. Inhibition of canonical NF-κB, in turn, diminishes production of chemotherapy-induced, inflammation and survival-promoting cytokines.

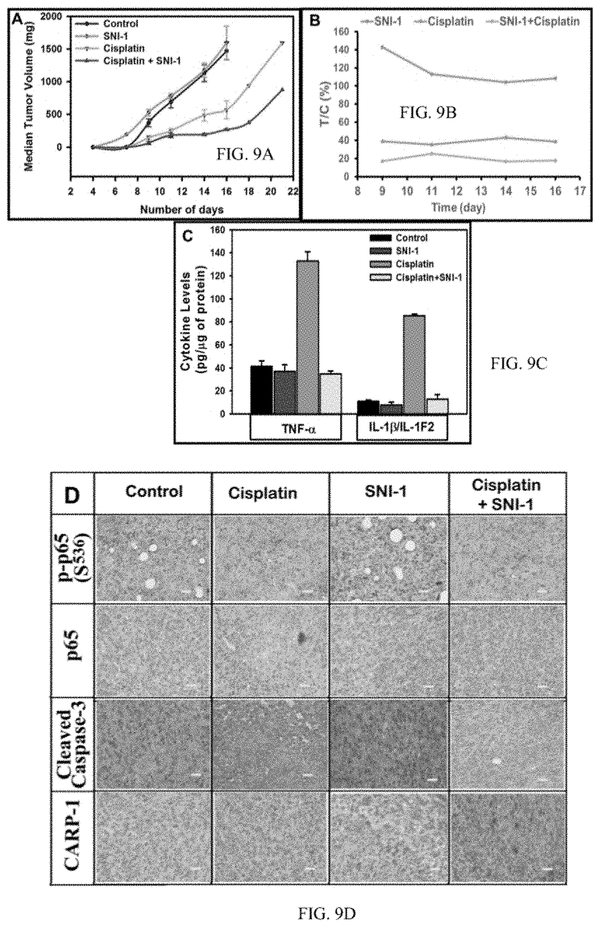

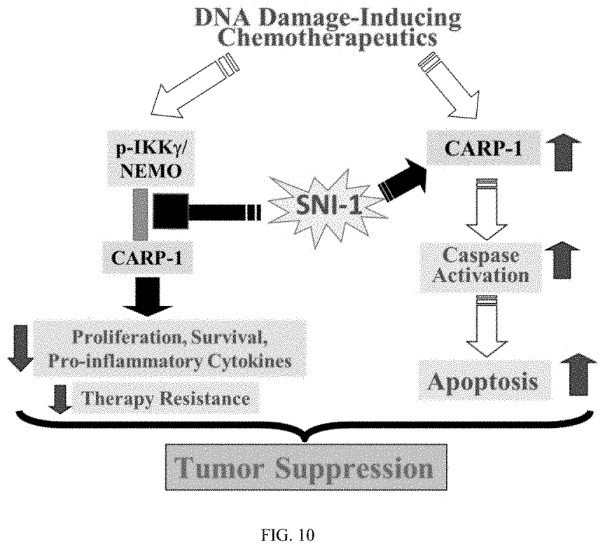

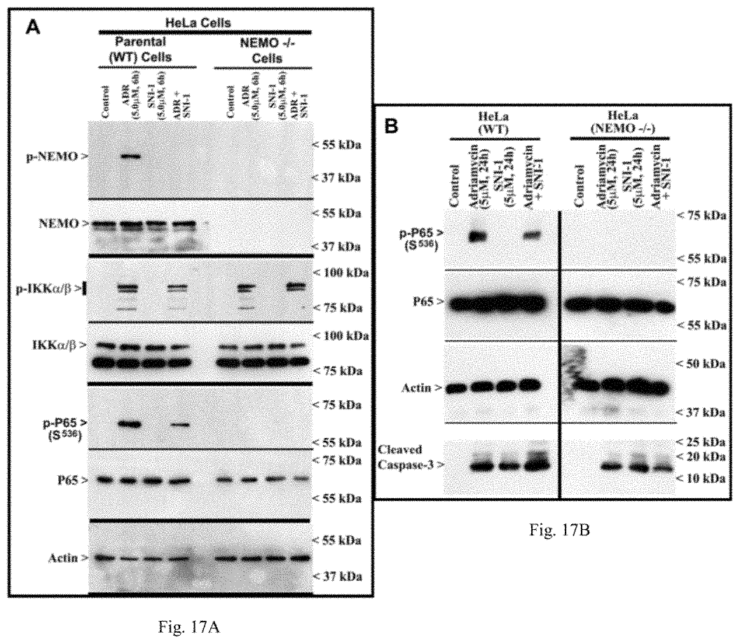

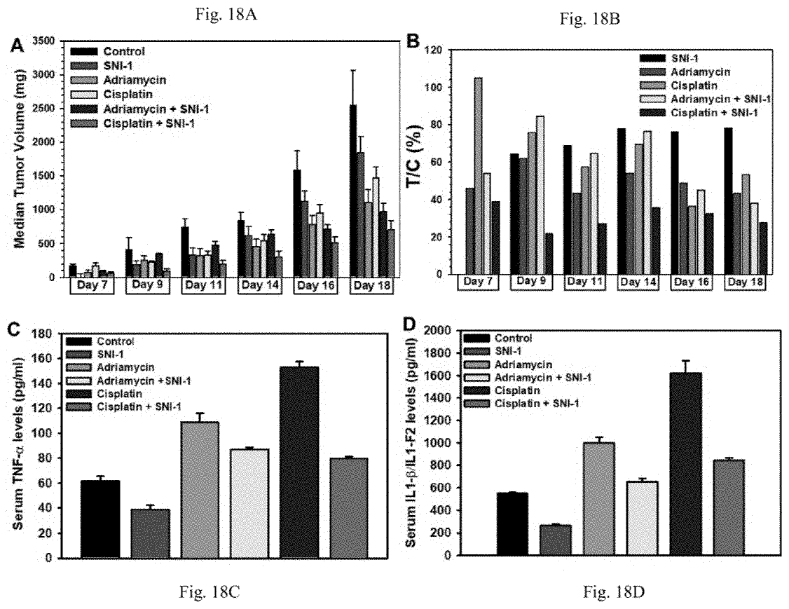

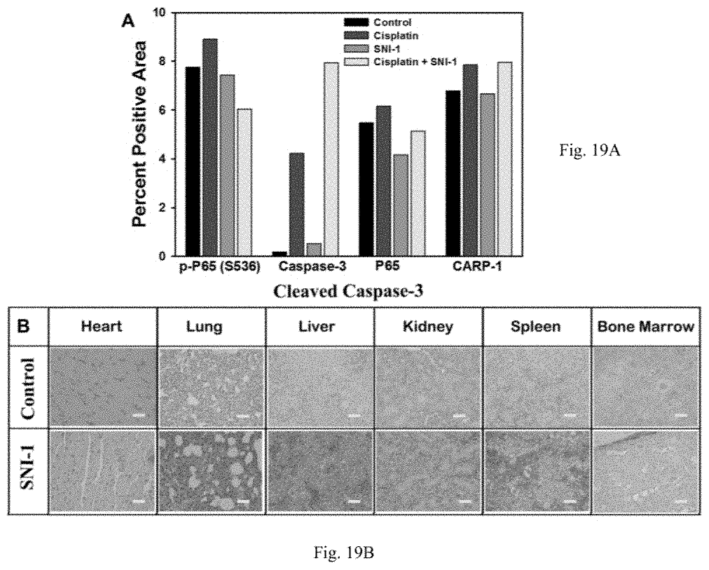

To investigate therapeutic potential of SNI-1, in vivo studies were conducted to determine efficacy and potency of SNI-1 alone or in combination with Adriamycin or Cisplatin (Cheriyan et al. (2016) Oncotarget 7, 73370-73388; and Cheriyan et al. (2017) Oncotarget 8, 104928-104945). As shown in FIG. 9 and FIG. 18, the treatment groups except the SNI-1 treatment group showed tumor growth inhibition as indicated by the reduced median tumor volume compared to the control group. With the exception of Cisplatin and Cisplatin plus SNI-1 groups, the groups treated with each of single agents, as well as a combination of SNI-1 and Adriamycin failed to reach effective T/C throughout the treatment period (FIG. 9, FIG. 18). Although a sustained and reduced median tumor volumes in Adriamycin plus SNI-1 and Cisplatin plus SNI-1 groups compared with respective single agent treated group was noted (FIG. 18B), Cisplatin plus SNI-1 treated group however reached a therapeutic T/C of <42% from day 7 until the end of treatment on day 18. In addition, consistent with the findings in FIGS. 8E and F, ELISA-based analyses revealed that treatments with Adriamycin or Cisplatin, but not SNI-1, robustly stimulated serum levels of pro-inflammatory cytokines TNFα and IL-1β (FIGS. 9C, 18C, 18D). Adriamycin and SNI-1 combination provoked a decline in serum levels of these cytokines when compared with their levels in sera derived from Adriamycin-treated animals (FIGS. 18C, 18D). A combination of SNI-1 and Cisplatin treatments, however, elicited a rather robust decline in serum levels of both TNFα and IL-1β when compared with their levels in sera derived from animals treated with Cisplatin alone (FIGS. 9C, 18C, 18D). Further, immuno-histochemical analyses of the tumors derived from animals treated with Cisplatin, but not SNI-1, revealed presence of phosphorylated p/65RelA, while a decline in the levels of phosphorylated p65 was noted in tumors derived from animals treated with Cisplatin plus SNI-1 (FIGS. 9S and 19A). Consistent with the in vitro data in FIG. 7, tumors derived from animals treated with Cisplatin, SNI-1, or a combination revealed strong presence of cleaved caspase-3 when compared with tumors derived from untreated control animal (FIG. 9D and FIG. 19A). Interestingly, and in contrast to the in vitro data with the cancer cell models, the in vivo studies revealed that although SNI-1 administration caused absent to minimal inhibition of tumor growth, and it did not provoke toxicities in the animals. While hematoxylin and eosin staining of various tissues including lungs, spleen, heart, liver, kidneys, and bone marrow of the animals treated with SNI-1 did not indicate microscopic alterations, immuno-histochemical staining of these tissues also failed to show presence of activated caspase-3 (FIG. 19B). The data in FIGS. 18, 19, and 9 collectively show that SNI-1 is likely safe, bioavailable with minimal to absent systemic toxicities. SNI-1 functions to enhance anti-tumor efficacy of Cisplatin in vivo, in part by robustly inhibiting p65/RelA activation, lowering systemic levels of pro-inflammatory cytokines, and inducing tumor apoptosis.

Discussion Described herein is the finding that CARP-1 is a regulator of canonical NF-κB signaling. CARP-1 regulates chemotherapy-induced, canonical NF-κB signaling in part by binding with the NEMO/IKKγ. Although, CARP-1 binding with NEMO was reported in a previous proteomic based study (Bouwmeester et al. (2004) Nat Cell Biol 6, 97-105), neither the molecular mechanism(s) nor the functional consequences of this interaction were elucidated. Here, mutagenesis-based studies were employed to define the molecular basis of this interaction. It was shown that CARP-1 amino acids 551-580 harbor the minimal epitope for its interaction with NEMO, while amino acids 221-261 of the NEMO protein contained the CARP-1-interacting epitope. CARP-1 interaction with NEMO was functionally significant because expression of CARP-1 (Δ553-599) interfered with activation of RelA by Adriamycin or CFM-4.16 compound but not TNFα, IL2-β, or EGF. Moreover, stable expression of CARP-1 (551-580; SEQ ID NO: 6) or NEMO (221-261; SEQ ID NO: 2) peptides that would in principle compete/interfere with binding of endogenous CARP-1 and NEMO proteins resulted in significantly higher loss of viabilities of cells treated with Adriamycin, Cisplatin, 5-FU, or CFM-4.16 compound.

Adriamycin or CFM-4.16 compound promote apoptosis in part by inducing DNA damage (Sekhar et al. (2019) Cancers (Basel) 11). The cellular DNA Damage Response (DDR) involves activation of ATM kinase and its down-stream target H2AX, and nucleus to cytoplasm activation of canonical NF-κB. For a robust DDR, NEMO first translocates to the nucleus where it is sumoylated, leading to NEMO nuclear retention. NEMO is then phosphorylated by ATM kinase, and then mono-ubiquitylated, followed by nuclear export of NEMO/ATM complex and activation of cytoplasmic IKK (Perkins, N. D. (2007) Nat Rev Mol Cell Biol 8, 49-62). Interestingly, DNA damage per se is not necessary for NEMO sumoylation. Other stress conditions, such as oxidative stress, ethanol exposure, heat shock and electric shock, also induce NEMO sumoylation (Wuerzberger-Davis et al. (2007) Oncogene 26, 641-651).

Although DNA damage-induced NEMO translocation to and from the nucleus is a hallmark of IKK activation in the canonical NF-κB pathway, the molecular mechanism(s) regulating nuclear-cytoplasmic shuttling of NEMO have yet to be fully clarified. In this regard, a recent report has revealed that IPO3 (aka importin 3, transportin 2, TRN2, or TNPO2), functions as an important NEMO nuclear import receptor during DDR (Hwang et al. (2015) J Biol Chem 290, 17967-17984). IPO3 facilitates NEMO nuclear translocation in a manner dependent on two, distinct nuclear localization signal (NLS) sequences in the human NEMO protein. Although human NEMO NLS 1 and 2 sequences have been mapped to positions 254-257 (SEQ ID NO: 25) and 357-360 (SEQ ID NO: 26), respectively, the murine NEMO protein harbors the NLS2 sequence and lacks NLS1 (Hwang et al. (2015) J Biol Chem 290, 17967-17984). The murine CARP-1 and NEMO proteins interact (FIG. 1A) and NEMO-binding epitopes of human and murine CARP-1 are identical (FIG. 12H). Further, since disruption of CARP-1 binding with NEMO by SNI-1 resulted in loss of chemotherapy-induced activation of p/65RelA (FIG. 7) in both human and murine cells, it collectively shows that CARP-1 binding with NEMO is independent of NEMO NLS1. Moreover, ATM kinase also rapidly translocates to nucleus following induction of DSBs that involves binding with importin α1/β1 heterodimer that is dependent on a distinct NLS in the ATM protein (Young et al. (2005) J Biol Chem 280, 27587-27594). A recent study further highlighted genotoxic stress-induced mono-ubiquitination of NEMO by an E3-ligase TRIM37 (Wu et al. (2018) Can Res 78, 6399-6412). Genotoxic stress-induced ATM activation resulted in phosphorylation of TRIM31 in cytosol, which induced a complex with TRAF6, and consequent nuclear import. Disruption of TRAF6 binding with TRIM31 resulted in increased sensitivity to chemotherapeutics in part due to diminished NEMO mono-ubiquitination in the nucleus. Whether CARP-1 also binds with ATM, TRAF6, TRIM31 or another E3 ligase is currently unclear. CARP-1 however directly binds with NEMO. Since CARP-1 is a perinuclear protein, it also not clear whether CARP-1 binding with NEMO functions to regulate nuclear import of NEMO following DNA damage. However, abrogation of CARP-1 binding with NEMO resulted in diminished NEMO phosphorylation (FIG. 3, FIG. 7). Since ATM kinase promotes serine 85 phosphorylation of NEMO in the nucleus following activation of DNA damage signaling, it is likely that CARP-1 binding with NEMO serves to facilitate ATM-dependent phosphorylation of NEMO. This possibility is also supported by WB data in FIG. 3 and confocal imaging (FIG. 13) where CFM-4.16 or Adriamycin-treated cells that express CARP-1 (Δ553-599) mutant had diminished NEMO serine 85 phosphorylation and cytoplasmic accumulation when compared with their similarly treated counterparts expressing wild-type CARP-1. Since NEMO phosphorylation often precedes its mono-ubiquitination, it is also unclear whether and to the extent CARP-1 binding with NEMO regulates NEMO mono-ubiquitination. Nevertheless, the current findings collectively support that CARP-1 binding with NEMO facilitates ATM-mediated NEMO phosphorylation and likely nuclear export of NEMO-ATM complex for activation of canonical NF-κB by genotoxic stress to modulate apoptotic response (Wu et al. (2006) Science 311, 1141-1146) and production of inflammatory cytokines that contribute to therapy resistance (Hwang et al. (2015) J Biol Chem 290, 17967-17984).

Although more than 700 different inhibitors (aspirin to IκBα super repressor) of NF-κB have been reported (Gilmore, T. D. and Herscovitch, M. (2006) Oncogene 25, 6887-6899), thus far no NF-κB blocker has been approved for human use. Given NF-κB's physiological roles in immunity, inflammation, and cellular homeostasis, a selective inhibition of therapy-induced NF-κB activation without affecting the immunity, inflammation, and homeostasis signaling would be desirable. Functional studies would then be performed for determining optimal regulator/transducer in this complex pathway, as well as identification of opportunities for synergistic agents to augment their efficacy and minimize resistance mechanisms. In this context, the results described herein provide evidence of selective activation of chemotherapy-dependent canonical NF-κB signaling by CARP-1-NEMO interactions. Moreover, a number of prior reports have highlighted targeting of NEMO for inhibition of inflammation regulated by canonical NF-κB signaling. For example, targeting of NEMO by endogenous proteins such as Hsp70 (Ran et al. (2004) Genes Dev 18, 1466-1481), cell permeable NEMO binding domain (NBD) peptides of IKKα and IKKβ (May et al. (2000) Science 289, 1550-1554; and Dai et al. (2004) J Biol Chem 279, 37219-37222), the peptides corresponding to the leucine zipper (LZ) and the coiled-coil 2 (CC2) regions of NEMO (Agou et al. (2004) J Biol Chem 279, 54248-54257) have been documented. Further, a small molecule that targets NEMO ubiquitin binding domain was recently reported (Vincendeau et al. (2016) Sci Rep 6, 18934). In addition, the medicinal compound Withaferin disrupted ubiquitin-based NEMO reorganization by regulating its covalent modification and binding with ubiquitin, while it also targeted IKKβ to inhibit NF-κB signaling and associated inflammatory responses (Hooper et al. (2014) J Biol Chem 289, 33161-33174; Jackson et al. (2015) Exp Cell Res 331, 58-72; and Heyninck et al. (2014) Biochem Pharmacol 91, 501-509).

The high-throughput chemical biological studies resulted in identification of small molecular compounds. The compound SNI-1 binds with CARP-1 and interferes with NEMO-CARP-1 interaction (FIG. 5). Although SNI-1 does not bind with NEMO, by disrupting CARP-1 binding with NEMO, it causes loss of chemotherapy-induced phosphorylation of NEMO. Since NEMO phosphorylation is often a prerequisite for NEMO ubiquitination and nuclear export to promote chemotherapy-induced activation of NF-κB, SNI-1 would not be expected to interfere with functions of cytoplasmic NEMO often necessary for activation of canonical NF-κB following growth factor or cytokine-dependent cellular homeostasis. In this regard, the data demonstrate that SNI-1 presence affects the chemotherapy-induced p65/RelA activation. Since chemotherapy often activates canonical NF-κB to promote survival and production of pro-inflammatory cytokines, presence of SNI-1 also attenuates secretion of chemotherapy-induced inflammatory cytokines in vitro as well as systemically in TNBC tumor-bearing animals in vivo. Of note here is that similar to chemotherapy, SNI-1 is able to inhibit cancer cell growth in vitro in part by inducing apoptosis as indicated by increasing levels of activated/cleaved caspase-3 (FIG. 7). Interestingly, and in contrast to the in vitro cell models, the in vivo studies revealed that although SNI-1 administration caused absent to minimal inhibition of tumor growth, and it did not provoke toxicities in the animals either. While hematoxylin and eosin staining of various tissues including lungs, spleen, heart, liver, kidneys, and bone marrow of the animals treated with SNI-1 did not indicate microscopic alterations, immuno-histochemical staining of these tissues also failed to show presence of activated caspase-3 (FIG. 19), albeit caspase-3 activation was noted in tissues derived from chemotherapy-treated animals. These findings collectively underscore a suitable safety profile of SNI-1 for further development and testing.

In summary, the results disclosed herein demonstrate that CARP-1 is an endogenous regulator of chemotherapy-induced canonical NF-κB activation. Pharmacological inhibition of CARP-1 binding with NEMO enhances chemotherapy efficacy in vitro and in vivo, in part by attenuating activation of canonical NF-κB, and secretion of NF-κB activated pro-inflammatory cytokines. SNI-1 represents a tool to investigate canonical NF-κB signaling with potential for translational development to target chemotherapy-induced cancer survival and resistance mechanisms.

Experimental Procedures. Materials: DMEM, EMEM medium and antibiotics (penicillin and streptomycin) were purchased from Invitrogen Co. (Carlsbad, Calif.). Fetal bovine serum (FBS) was purchased from Denville Scientific Inc. (Metuchen, N.J.), and DMSO was purchased from Fisher Scientific (Fair Lawn, N.J.). Chemi-luminescence Reagent was purchased from Amersham Biosciences (Piscataway, N.J.), and the Protein Assay Kit was purchased from Bio-Rad Laboratories (Hercules, Calif.). Structure and synthesis of CFM-4 analog CFM-4.16 has been described (Cheriyan et al. (2016) Oncotarget 7, 73370-73388). Clinical grade ADR, Cisplatin, Oxaliplatin and 5-fluouracil were obtained from the Harper Hospital Pharmacy, Wayne State University, Detroit, Mich. The Selective NF-κB Inhibitor (SNI)-1 and 2 compounds 1-(3,4-dihydroxyphenyl)-2-{(1-(4-methylphenyl)-1H-tetrazol-5-yl)thio}ethanone, and 2-{[(4-methoxyphenyl)sulfonyl)amino}-N-(2-phenylethyl]benzamide, respectively, that inhibited CARP-1/NEMO binding in the HTS were purchased from ChemBridge, San Diego, Calif. SNI-1 compound of >98% purity was also synthesized by Otava Chemicals, Toronto, Canada. 5-dimethyltiazol-2-yl-2.5-diphenyl-tetrazolium bromide (MTT), anti-FLAG tag and anti-actin antibodies were purchased from Sigma Chemical Co, St. Louis, Mo. The affinity purified, anti-CARP-1 (α1 and α2) polyclonal antibodies have been described (Rishi et al. (2003) J Biol Chem 278, 33422-33435). Anti-EGFP and phospho (S85) NEMO antibodies were purchased from Abcam, Cambridge, Mass., while anti-HA-tag antibodies were purchased from Biolegend, San Diego, Calif. Antibodies for Gst-tag, Myc-tag, 6×His-tag, total NEMO, phospho (5536) and total p65RelA, phospho (S176/S180) IKKα/β and total IKKβ, phospho and total JNK/2, phospho (Y705) and total STAT3, and RIPK were purchased from Cell Signaling, Beverley, Mass.

Recombinant plasmid constructs: The plasmids for expression of myc-His-tagged wild-type CARP-1 (clone 6.1.2), CARP-1 (Δ600-650; SEQ ID NO: 21), CARP-1 (1-198; SEQ ID NO: 27), CARP-1 (197-454; SEQ ID NO: 28), CARP-1 (452-654; SEQ ID NO: 18), CARP-1 (603-898; SEQ ID NO: 29), CARP-1 (896-1150; SEQ ID NO: 30) have been described (Rishi et al. (2003) J Biol Chem 278, 33422-33435; Rishi et al. (2006) J Biol Chem 281, 13188-13198; and Sekhar et al. (2019) Cancers (Basel) 11). Additional pcDNA-based plasmids for expression of myc-His-tagged CARP-1 (Δ553-599; SEQ ID NO: 1), CARP-1 (Δ521-566; SEQ ID NO: 20), CARP-1 (452-625; SEQ ID NO: 31), CARP-1 (452-610; SEQ ID NO: 32), CARP-1 (452-552; SEQ ID NO: 33), CARP-1 (552-654; SEQ ID NO: 10), CARP-1 (552-640; SEQ ID NO: 34), CARP-1 (552-625; SEQ ID NO: 35), CARP-1 (552-610; SEQ ID NO: 36), CARP-1 (552-580; SEQ ID NO: 22), CARP-1 (571-600; SEQ ID NO: 37), CARP-1 (591-620; SEQ ID NO: 38), pcDNA3-EGFP, pcDNA3-EGFP CARP-1 (551-580; SEQ ID NO: 6), pcDNA3-Gst, pcDNA3-Gst-NEMO, pcDNA3-Gst-NEMO (221-261; SEQ ID NO: 2), pcDNA3-Gst-NEMO (Δ221-258; SEQ ID NO: 23) were generated by standard molecular biological and cloning manipulations. Plasmids encoding wild-type and mutant NEMO proteins with 6×myc epitopes at the amino terminus have been described (Huang, T. T., et al. (2003) Cell 115, 565-576). These plasmids encoded NEMO ΔN120 (lacking N-terminus 220 amino acids), NEMO ΔC125 (lacking C-terminus 25 amino acids), NEMO C417R, and NEMO D406V mutant proteins. Recombinant plasmids encoding Gst-tagged NEMO (wild-type), NEMO (2-260; SEQ ID NO: 3), NEMO (221-261; SEQ ID NO: 2), NEMO (221-317; SEQ ID NO: 39), and NEMO (296-419; SEQ ID NO: 40) were generated by PCR amplification of NEMO cDNA fragments and their subsequent subcloning in the pEBG vector plasmid. The NEMO (2-260; SEQ ID NO: 3) cDNA was cloned in pGEX-4T-1 vector to generate bacterial (E. coli) expressed Gst-NEMO (2-260; SEQ ID NO: 3) protein. Additional CARP-1 cDNA fragments were cloned in pTAT-HA vector (Zhang et al. (2007) Mol Cancer Ther 6, 1661-1672) to generate bacterial (E. coli) expressed His-TAT-HA-tagged CARP-1 (552-654; SEQ ID NO: 10), CARP-1 (552-580; SEQ ID NO: 22), CARP-1 (571-600; SEQ ID NO: 37), CARP-1 (591-620; SEQ ID NO: 38), CARP-1 (611-640; SEQ ID NO: 41), and CARP-1 (631-660; SEQ ID NO: 42) proteins. The NF-κB TATA-Luc plasmid harboring 5×NF-κB consensus enhancer sequences positioned upstream of TATA sequences that collectively drive firefly luciferase reporter as well as the plasmid for expression of Renilla luciferase (pTK/Renilla Luc) were purchased from Stratagene, Inc. (LaJolla, Calif.) and Promega, Inc. (Madison, Wis.), respectively. The recombinant plasmids were sequenced to confirm the accuracy and validity of various inserts/epitopes.

Cell Lines and Cell Culture: Routine maintenance and culture of MDA-MB-468 and MDA-MB-231 (both lack estrogen receptor and have mutant p53), SUM-149, SUM-1315, and HCC1937 (these three have mutant BRCA1) human TNBC, human Cervical Cancer HeLa, human pancreatic cancer PANC-1, human diffuse large B-cell lymphoma WSU-DLCL2, human follicular lymphoma WSU-FSCCL, human clear cell renal carcinoma A498, human colon cancer HT-29, SW620, HCT-116, HCT-116 (P53−/−), colon epithelial IEC-6 cells, and monkey kidney COS-7 cells was carried out as described (Rishi et al. (2003) J Biol Chem 278, 33422-33435; Rishi et al. (2006) J Biol Chem 281, 13188-13198; Sekhar et al. (2019) Cancers (Basel); Puliyappadamba et al. (2011) J Biol Chem 286, 38000-38017; and Cheriyan et al. (2017) Oncotarget 8, 104928-104945). HeLa and MDA-MB-468 cells having Crisper-based CARP-1 knock-out were generated and characterized by Biocytogen Corp., Wakefield, Mass. HeLa cells having Crisper-based NEMO knock-out have been described (Fang et al. (2017) J Immunol 199, 3222-3233). The murine TNBC cell line 4T1 that was derived from a spontaneously arising BALB/c mammary tumor were obtained from the Karmanos Cancer Institute (KCl), and were maintained in culture (Cheriyan et al. (2016) Oncotarget 7, 73370-73388). Generation, characterization, and culture of drug (ADR or Cisplatin)-resistant human TNBC MDA-MB-468 and MDA-MB-231 cells as well as ADR-resistant murine 4T1 cells have been detailed (Cheriyan et al. (2016) Oncotarget 7, 73370-73388). The cell culture media were also supplemented with 10% FBS, 100 units/ml of penicillin, and 100 μg/ml of streptomycin, and the cells were maintained at 37° C. and 5% CO₂. For cell growth and MTT studies, the cells were cultured in fresh media with 5%-10% FBS prior to their treatments with various agents. Generation and characterization of MDA-MB-468 cells expressing reduced CARP-1 has been described (Rishi et al. (2003) J Biol Chem 278, 33422-33435). The stable sublines were generated by transfecting the MDA-MB-468 and Hela cells with the pcDNA3 vector, pcDNA3-CARP-1 (WT), pcDNA3-EGFP, pcDNA3-Gst, and various Myc-His, EGFP, or Gst tagged mutants of CARP-1 as well as NEMO followed by selection in the presence of 800 μg/ml neomycin using described methods (Rishi et al. (2003) J Biol Chem 278, 33422-33435; Rishi et al. (2006) J Biol Chem 281, 13188-13198; Sekhar et al. (2019) Cancers (Basel); Puliyappadamba et al. (2011) J Biol Chem 286, 38000-38017; and Cheriyan et al. (2017) Oncotarget 8, 104928-104945). The cell lysates from wild-type, untransfected cells, neomycin-resistant pools, or individual sublines were then subjected to IP and WB analyses. Two, well characterized TNBC patient-derived (PDX) tumors (TM00089, -091) were purchased from JAX labs, and were routinely maintained/passaged.

Three-dimensional Mammosphere Assays: The PDX tumor cells were dissociated from the tumor fragments, and cultured for 2-dimensional and mammosphere studies in vitro (Cheriyan et al. (2016) Oncotarget 7, 73370-73388). Briefly, the cells were washed twice in 1×PBS, trypsinized, and pelleted at 200×g at room temperature. Cells were then re-suspended in 5 ml of mammosphere media (DMEM/F12 containing 2 mM L-glutamine, 100 U/ml penicillin, 100 U/ml streptomycin, 1×B27 supplement, 20 ng/ml recombinant human epidermal growth factor (EGF; Sigma), 10 ng/ml recombinant human basic fibroblast growth factor (bFGF; R&D Systems). Approximately 5000 viable cells per ml was then seeded in an ultra-low adherent 60 mm plate and incubated at 37° C. and 5% CO₂ for two weeks without disturbing the plates. After the mammospheres formed, fresh media with or without Adriamycin (5 μM), SNI-1 (2.5 or 5.0 μM), or a combination of these agents was added and the cells incubated for additional 48 h at 37° C. and 5% CO₂. The mammospheres in the untreated and treated plates were photographed, and the cells were then dissociated to determine their viabilities by the MTT assay (Cheriyan et al. (2016) Oncotarget 7, 73370-73388).

Cell Viability, Immunoprecipitation and Western Blot assays: 500-1000 cells were seeded in each well of 96 well plate and then either untreated (control) or treated with various agents for noted times. After treatment, MTT reagent was added at 0.5 mg/ml concentration for 2-4 hours at 37° C. DMSO was added to solubilize formazan and the plate was read at 570 nm in a plate reader. The histograms indicating levels of cell viability were generated by plotting the net absorbances (Rishi et al. (2003) J Biol Chem 278, 33422-33435; and Rishi et al. (2006) J Biol Chem 281, 13188-13198). Next, logarithmically growing cells were either untreated or treated with different agents for various time periods. The cells were lysed to prepare protein extracts. Immuno-precipitation (IP) was carried out by incubating approximately 1 mg of the protein lysate with appropriate antibodies. For Gst-pulldown, Gst-NEMO or various His-TAT-HA-tagged CARP-1 peptides were generated in E. coli BL21 cells (Zhang et al. (2005)Mol Cancer Ther 6, 1661-1672; and Sekhar et al. (2019) Cancers (Basel) 11). Briefly, bacterial pellet was lysed in 100-200 microliters of BPER buffer (Thermofisher) with DNAseI at RT, and supernatant checked for expression of respective fusion peptides by WB. Following confirmation of expression, 5-20 μl of lysate expressing Gst fusion protein was first incubated with 20 μl of precleared glutathione sepharose in a final volume of 100 μl at 4° C. for 2 h with constant rotation. The sepharose beads were spun at 800×g for 2 min, and the pellet was washed two-four times with 100-200 microliters of RIPA buffer with 0.5M NaCl. The beads were spun again as above, and mixed with 5-20 μl of E. coli lysate expressing His-TAT-HA CARP-1 peptides. The reactions were incubated further at 4° C. for 2 h with constant rotation. The peptide-bound sepharose beads were pelleted and washed with 100-200 μl of RIPA buffer with 0.1M NaCl for 2-4 washes. If necessary, additional washes with 0.05M NaCl buffer were carried out. In some instances, the complexes were incubated with a small molecule compound followed by additional NaCl washes as described herein. After the final wash, the sepharose-protein complexes were spun, and then re-suspended in SDS loading buffer for electrophoresis on 12-15% SDS PAGE, followed by WB with appropriate antibodies. Alternatively, a similar pull-down strategy was carried out by immobilizing the His-TAT-HA peptides on the Ni-NTA matrix, followed by washing, incubation with a small molecule compound and/or E. coli lysates with Gst-tagged proteins, SDS-PAGE analysis, and WB with anti-Gst tag antibodies. Luciferase assays were performed as described (Muthu, M., et al. (2014) PLoS One 9, e102567). Briefly, 3×10⁵ cells in culture media minus FBS were plated in 12- or 24-well plate, and transfected with a combination of pTK/Renilla Luc and NF-κB-TATA-Luc plasmids. Five hours post-transfection, FBS was added to media, cells were allowed to grow for 18 h. Cells were then treated with DMSO (Control), Adriamycin, SNI-1, or a combination, for 1 h, harvested, lysed, and Renilla and firefly luciferase activities were measured using dual luciferase assay kit (Promega) following vendor's guidelines.

Immunofluorescence staining and confocal microscopy: Cells were plated onto chamber slides 24 h prior to treatment. Following treatment of cells with respective compounds, the adherent cells were fixed with 5% formaldehyde for 10 min and then washed with PBS. Samples were blocked (0.5% NP-40, 5% milk powder, 1% fetal bovine serum) for 30 min. After a single wash with PBS, cells were incubated with primary antibodies for 45 to 60 min. Cells were washed with PBS and then incubated with secondary antibodies for another 45 to 60 min, followed by washing with PBS and mounting with 0.1 μg/ml DAPI (4′,6′-diamidino-2-phenylindole) containing mounting solution. For confocal imaging, cells were first fixed with PFA, stained for CARP-1 by myc-tag antibodies (green), NEMO by phospho-NEMO antibodies (red), and DAPI (blue) for nuclear staining. Immuno-fluorescent or confocal images were taken using Zeiss LSM 510 Meta NLO (63X) (Sekhar et al. (2019) Cancers (Basel) 11).

Cytokine ELISA assays: Secretion of pro-inflammatory cytokines TNFα, IL8, and IL-1β in untreated and treated human TNBC, pancreatic, and renal cancer cells, and mouse 4T1 TNBC cells as well as in sera of 4T1 tumor bearing Balb/c mice was quantitatively measured by 96-well, Quantikine colorimetric ELISA-based assays were carried out following manufacturer (R&D Systems, Minneapolis, Minn.) suggested methods and guidelines.

Kinetics of CARP-1-NEMO interaction: In the absence of available X-ray crystal structures for CARP1, homology modeling was performed on its known sequence in order to build a suitable protein model (Sekhar et al. (2019) Cancers (Basel) 11). Briefly, SWISS-MODEL (Waterhouse et al. (2018) Nucleic Acids Res 46, W296-w303) was used to build homology models for CARP1 (551-600; SEQ ID NO: 5) that harbors epitope for interaction with NEMO. A crystal structure for NEMO has been elucidated, and thus the structure of NEMO (221-261; SEQ ID NO: 2) that interacts with CARP1 was obtained from the PDB (3CL3) (Bagneris et al. (2018) Mol Cell 30, 620-631). Protein-protein docking was performed using ZDOCK 3.0.2f with IRaPPA re-ranking (Pierce, B. G., et al. (2014) Bioinformatics 30, 1771-1773). The top three predictions for each complex were further subjected to molecular dynamics (MD) using the AMBER14 package to relieve clashes resulting from docking (Heyninck et al. (2014) Biochem Pharmacol 91, 501-509). MD calculations were conducted with a 24 ns production run for each complex (Sekhar et al. (2019) Cancers (Basel) 11).

Next, kinetics of CARP-1 binding with NEMO were determined by Surface Plasmon Resonance (SPR) Technology (Profacgen, Shirley, N.Y.). Briefly, CARP-1 (551-580) peptide (Amino terminal-HRPEETHKGRTVPAHVETVVLFFPDVWHCL-Carboxyl terminal; SEQ ID NO: 6) was dissolved in water, and various concentrations of CARP-1 Peptide were manually printed onto the bare gold-coated (thickness 47 nm) PlexArray Nanocapture Sensor Chip (Plexera Bioscience, Seattle, Wash., US) at 40% humidity. Each concentration was printed in replicate, and each spot contained 0.2 μL of sample solution. The chip was incubated in 80% humidity at 4° C. for overnight, and rinsed with 10×PBST for 10 min, 1×PBST for 10 min, and deionized water twice for 10 min. The chip was then blocked with 5% (w/v) non-fat milk in water overnight, and washed with 10×PBST for 10 min, 1×PBST for 10 min, and deionized water twice for 10 min before being dried under a stream of nitrogen prior to use. The binding reactions with NEMO (221-260; SEQ ID NO: 7) peptide (Amino terminal-EEKRKLAQLQVAYHQLFQEYDNHIKSSVVGSERKRGMQLE-Carboxyl terminal; SEQ ID NO: 7) were performed in PBST buffer (0.01M phosphate buffered saline (0.138 M NaCl; 0.0027 M KCl), 0.05% Tween-20, pH 7.4). SPRi measurements were performed with PlexAray HT (Plexera Bioscience, Seattle, Wash., US). Collimated light (660 nm) passes through the coupling prism, reflects off the SPR-active gold surface, and is received by the CCD camera. Buffers and samples were injected by a non-pulsatile piston pump into the 30 μL flowcell that was mounted on the coupling prism. Each measurement cycle contained four steps: washing with PBST running buffer at a constant rate of 2 μL/s to obtain a stable baseline, sample injection at 5 μL/s for binding, surface washing with PBST at 2 μL/s for 300 s, and regeneration with 0.5% (v/v) H₃PO₄ at 2 μL/s for 300 s. The measurements were performed at 25° C. The signal changes after binding and washing (in AU) were recorded as the assay value. Selected protein-grafted regions in the SPR images were analyzed, and the average reflectivity variations of the chosen areas were plotted as a function of time. Real-time binding signals were recorded and analyzed by Data Analysis Module (DAM, Plexera Bioscience, Seattle, Wash., US). Kinetic analysis was performed using BIAevaluation 4.1 software (Biacore, Inc.).

Association and dissociation rate constants were calculated by numerical integration and global fitting to a 1:1 interaction model and the equation: dRU(t)/dt=kₐC(R^{max}−RU(t))−k_{d}RU(t), where RU(t) is the response at time t, Rₘₐₓ is the maximum response, C is the concentration of analyte in solution, kₐ is the association rate constant, k_{d} is the dissociation rate constant, and RU (0)=0.

The AlphaLISA assay for high throughput screening: For screening of a library of chemical compounds, an ELISA-based assay was developed and optimized for use in 384-well format (SAMDI Tech, Chicago, Ill.). The assay development involved buffer optimization by testing peptide binding in PBS, PBS+0.01% Tween, PBS+0.01% BSG, PBS+0.01% Tween, 0.01% BSG, and a proprietary buffer #79389 (BPS Bioscience, San Diego, Calif.). The assay utilized streptavidin donor and anti-FLAG acceptor beads (PerkinElmer, Shelton, Conn.) in conjunction with Flag-tagged CARP-1 (546-580; SEQ ID NO: 8) and biotin-tagged NEMO (221-261; SEQ ID NO: 2) peptides that were chemically synthesized to >95% purity (Peptides America, Fairfax, Va.). The peptides were dissolved in water, and the binding reactions consisted of 100 nM Flag-tagged CARP-1 (546-580; SEQ ID NO: 8) with 1000, 500, 250, 125, 62.5, 31.25, 15.125, or 0 nM of biotin-tagged NEMO (221-261; SEQ ID NO: 2) peptide in BPS buffer. The reaction was carried out at room temperature for a 60 min incubation of the peptide pair, followed by 0, 30, 90 minute incubation with AlphaLisa beads. In addition, binding reaction containing different concentrations of biotin-tagged NEMO (221-261; SEQ ID NO: 2) peptide was incubated with 100 nM of Flag-tagged CARP-1 (546-580; SEQ ID NO: 8) peptide in the absence or presence of 2.5% DMSO. The assay signal (Fluorescence) was measured at 680 nm excitation and 615 nm emission wavelengths to determine assay robustness and DMSO tolerance. Next, 10,240 total compounds from a Chembridge diversity set were screened in pools of 8 (5 μM final concentration) with a final concentration of 1% DMSO, 100 nM each peptide utilizing 384-well OptiPlates. The plates were read on a Pherastar FS plate reader. Positive control wells were absent compound and negative controls were run absent the biotinylated peptide. Hits were identified as those wells showing a % inhibition >3 standard deviations from the average inhibition across the plate. Those wells were further analyzed to investigate the 8 compounds individually in duplicate. The hits revealed during this confirmation step were then analyzed in a dose response experiment with 50 μM top concentration of compound with a 3-fold 10-point dilution series in duplicate.

Establishment of TNBC cell-derived xenografts in Syngeneic mice: Generation of 4T1 TNBC cell-derived sub-cutaneous xenografts in Balb/c mice were performed (Cheriyan et al. (2016) Oncotarget 7, 73370-73388; and Cheriyan et al. (2017) Oncotarget 8, 104928-104945). BALB/cAnNCr, 6-8-weeks old, female mice were purchased from Charles River Laboratories (Horsham, Pa.). Following suitable acclimation of animals, 1×10⁶ 4T1 TNBC cells were re-suspended in 200 μl of sterile saline, and implanted in the flanks using a 27-gauge needle. Tumors were allowed to grow to 500-1000 mg (˜10 days), and the aseptically harvested, minced into 3-4 mm³ (30 mg) fragments and transplanted SC into naïve recipient mice using a standard 12 gauge trocar to serially maintain the tumor in vivo. For efficacy studies, tumors serially maintained in vivo were aseptically harvested and minced into 3-4 mm³ (30 mg) fragments, then bilaterally transplanted subcutaneously along the flanks using a standard 12 gauge trocar. Mice were randomly assigned to control or one of five treatment groups (n=6 mice/group as follows: no treatment; SNI-1 (70 mg/kg/dose; QD 1-13 via intraperitoneal injection; total dose 910 mg/kg); Adriamycin (ADR; 4 mg/kg/dose; D1, 5, 10, 14 via intravenous injection; total dose 16 mg/kg); Cisplatin (CIS; 3 mg/kg/dose; D1, 5, 10, 14 via intravenous injection; total dose 12 mg/kg); and SNI-1+ADR or SNI-1+CIS on matching respective single arm schedules. For the combination arm, SNI-1 was administered first, followed within 1 hour by either ADR or CIS. A dose-route determination conducted with SNI-1 in non-tumor-bearing mice using a solubilized formulation (5% DMSO, 5% ethanol, 2% Tween 80 (v/v) with double-distilled water) found that the compound was not suitable for chronic intravenous administration. For the efficacy studies, SNI-1 was formulated in 8% DMSO (v/v) and 8% Cremophor (v/v) in cell grade water, pH4 as diluent.

Clinical grade ADR stock (2 mg/mL) was diluted to the appropriate concentration with cell grade water; pH4 and clinical grade CIS (1 mg/ml) diluted with USP 0.9% saline; pH6). Mice were monitored daily for changes in condition and body weight. Tumors were measured three times weekly by caliper and tumor volume (mg) was calculated using the following formula: (A×B2)/2 where A and B are the tumor length and width (in mm), respectively. Endpoints for assessing antitumor activity consisted of qualitative determinations via tumor growth inhibition (% T/C) where T is the median tumor volume of treated mice and C is the median tumor volume of control mice on any given day of measurement. According to NCI-accepted criteria, a treatment is considered effective if the T/C is <42%. Highly active agents produce T/C values <20%. Efficacy was also assessed quantitatively using tumor growth delay (T−C) defined as the difference between the median time (in days) required for the treatment group tumors (T) to reach 1000 mg and the median time (days) for the control group tumors to reach the same volume. Post last treatment, tumor tissue and samples from various organs (spleen, liver, kidney, heart, bone, and lungs) were collected. Additionally, the whole blood samples were obtained from a representative tumor-bearing mouse from each group via terminal cardiac puncture. Immuno-histochemical analyses of the tumor and tissue samples were performed for expression of activated RelA (serine 536 phosphorylated p65), total RelA, cleaved caspase-3, and CARP-1 proteins. The levels of pro-inflammatory cytokines in the sera were analyzed by sandwich ELISA assays following methods detailed herein (Cheriyan et al. (2016) Oncotarget 7, 73370-73388; and Cheriyan et al. (2017) Oncotarget 8, 104928-104945).

Statistical Analyses: The statistical analyses were performed using Prism 6.0 software. The data were expressed as mean SEM and analyzed using two-tailed student t-test or one-way ANOVA followed by a post hoc test. A p value of <0.05 was considered statistically significant.

The abbreviations used are: NF-κB, nuclear factor-kappa B; TNFα, Tumor necrosis factor α; IL-8, Interleukin-8; IL-1β, Interleukin 1; 5-FU, 5-fluouracil; CARP-1, cell cycle and apoptosis regulatory protein 1; CDKI, cyclin-dependent kinase inhibitor; IKK, Inhibitory kappa B kinase; NEMO, NF-κB essential modulator (aka, IKKγ); eGFP, enhanced green fluorescent protein; GST, glutathione S-transferase; FBS, fetal bovine serum; TAT, trans-activation of transcription tag; HBC, human breast cancer; TNBC, Triple-negative breast cancer; SNI-1, selective NF-κB inhibitor-1; ADR, Adriamycin/Doxorubicin.

### Example 2: Structures and In Vitro Activities of SNI-1 Analogs Including the Water Soluble Di-Sodium SNI-1

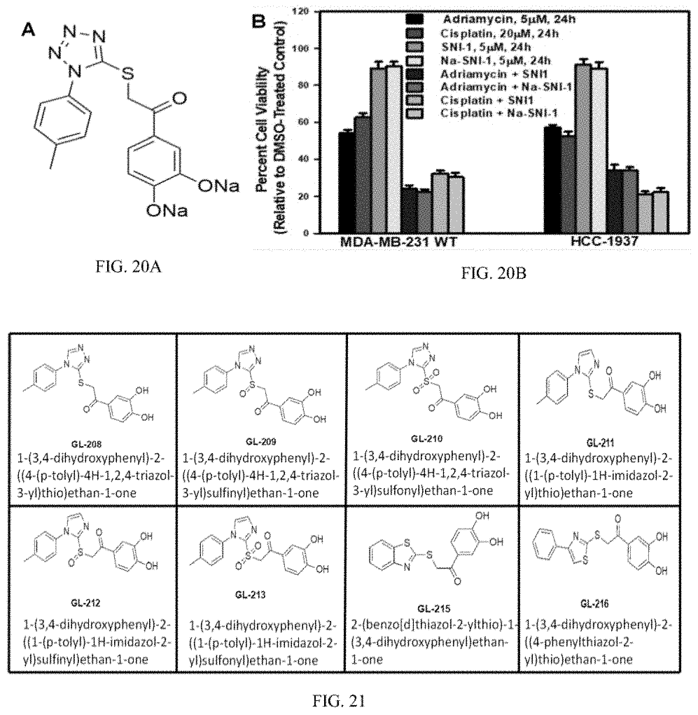

A water-soluble, di-sodium salt of SNI-1 as well as several analogs of SNI-1 were synthesized using medicinal chemistry methods/approaches. The structure of di-sodium SNI-1 and its biological activity data in vitro is show in FIG. 20. These data show that di-sodium salt of SNI-1 has similar activity as the parental SNI-1.

FIG. 21 shows chemical structures of additional SNI-1 analogs that were synthesized. MDA-MB-468 TNBC cells were next treated with each of the analog in vitro as a single agent or in combination with Adriamycin in vitro. The MTT data in FIG. 22A show that none of the analogs caused cell growth inhibition when used as single agents. In addition, SNI-1 in combination with Adriamycin caused greater loss of cell viability when compared with that noted for the cells treated with Adriamycin alone. Analogs GL-209, GL-213, and GL-216 also caused loss of cell viabilities that were greater than those noted in the case of cells treated with Adriamycin alone (FIG. 22B). Of note is that a decrease in viabilities of cells treated with Adriamycin in combination with GL-209, GL-213 or GL216 were comparable to that observed for cells treated with Adriamycin plus SNI-1. Additionally, a combination of Adriamycin and analogs GL-212 or GL-215 caused a moderate reduction in cell viabilities when compared with Adriamycin-treated cells, while GL-208, -210, or -211 compounds in combination with Adriamycin caused a minimal reduction in cell viabilities relative to cells treated with Adriamycin alone (FIG. 22B). These data collectively suggest that the disclosed compounds can be can be suitable sensitizers of chemotherapeutic Adriamycin.

### Example 3: In Vivo Activities of SNI-1 Analogs Including the Water Soluble Di-Sodium SNI-1

As disclosed herein, in a syngeneic 4T1 TNBC tumor model, a combination of SNI-1 and DNA Damage-inducing chemotherapeutics (Adriamycin or Cisplatin) caused a superior inhibition of tumor growth when compared with tumor growth inhibition noted in mice treated with either agent alone. Of note is that SNI-1 in combination with Adriamycin provoked a greater inhibition of tumor growth when compared with the tumor growth inhibition noted in animals treated with either agent alone. Interestingly, a rather robust inhibition of tumor growth was observed in the animals that were treated with SNI-1 and Cisplatin in combination compared to the tumor growth inhibition observed in animals treated with either SNI-1 or Cisplatin. Although both chemotherapy drugs, Adriamycin and Cisplatin, function in part by inducing DNA damage that involve distinct as well as overlapping mechanisms, the precise reason(s) for a robust tumor growth inhibition by Cisplatin and SNI-1 combination is unclear. Since TNBCs and many other cancers are often known to express drug efflux pumps that contribute to lower efficacy of drugs in clinic and development of drug-resistant tumors, it is likely that the presence/expression of these drug efflux pumps function in part to interfere with a robust anti-tumor response to a combination of SNI-1 and Adriamycin. In this regard, it was found that a robust increase in levels of drug efflux pump MDR1 in human TNBC MDA-MB-468 cells that were treated with Cisplatin, or SNI-1, while Adriamycin treatment provoked a rather moderate increase in MDR1 levels. It is unclear whether SNI-1 is also a substrate for MDR1, since Adriamycin (but not Cisplatin) is a substrate for MDR1 pump, it could explain in part a moderate Ariamycin+SNI-1 response in vivo. In light of a superior tumor growth inhibition by Cisplatin and SNI-1 combination, it was investigated whether a combination of SNI-1 and Cisplatin will cause a superior tumor growth inhibition in vivo in other syngeneic models of breast and renal cancers.

Mammary cell 16/C/Adriamycin Syngeneic TNBC Tumor Model: In a strategy similar to the experiments performed with 4T1 TNBC syngeneic tumor model, the effectiveness of Cisplatin and SNI-1 as a combination therapy was investigated utilizing the Main 16/C/Adr syngeneic TNBC tumor model (Corbett T H, et al. Transplantable Syngeneic Rodent Tumors: Solid Tumors of Mice. In: B. Teicher (ed). Tumor Models in Cancer Research Humana Press Inc., Totowa, N.J. Chapter 3, pp. 41-71, 2002). Of note is that the tumors in this model are resistant to taxol while being moderately sensitive to Adriamycin, or Cisplatin. As shown in FIG. 23, SNI-1+Cisplatin provoked a therapeutic response superior to single agent treatment in the Main 16/C/Adr tumor model.

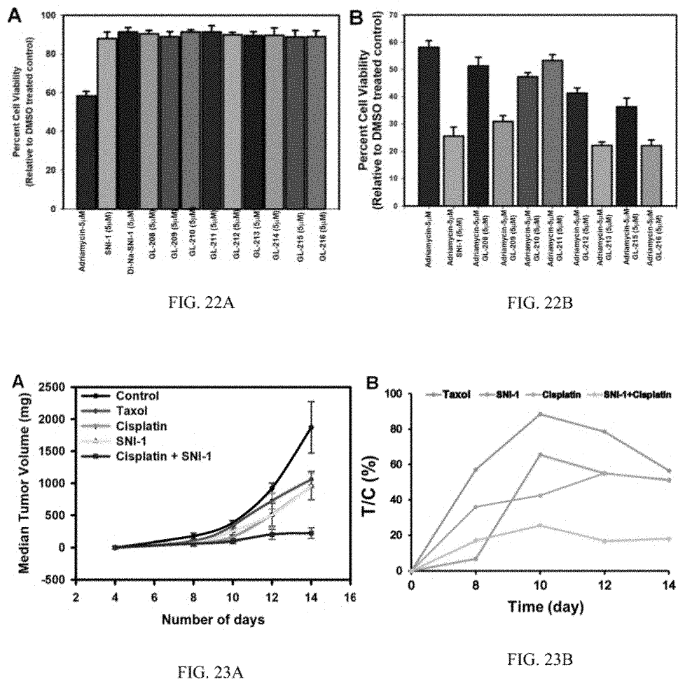

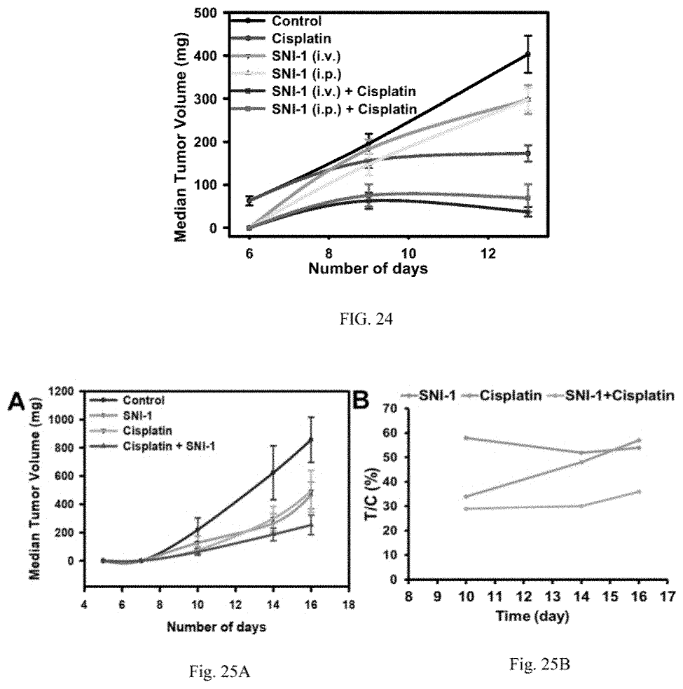

BRCA1 mutant TNBC Tumor Model: Testing for germline BRCA1/2 mutations permits identification of BRCA1/2 status as clinically relevant in the selection of therapy for patients already diagnosed with breast cancer. BRCA status predicts responsiveness to platinum-based chemotherapy, as well as to inhibitors of poly(ADP-ribose) polymerase (PARP) in breast and ovarian cancers. This is because Cisplatin and PARP inhibitors function in part by blocking DNA repair pathways (Tung N M, Garber J E. British Journal of Cancer 119, 141-152, 2018). On the basis of this rationale, SUM149 human BRCA1-mutant TNBC model was used to investigate the effectiveness of Cisplatin and SNI-1 combination therapy in an efficacy study. A related objective of this pilot study was to determine whether and to the extent SNI-1 or its water soluble, di-sodium derivative will elicit effects in vivo. NCr-SCID mice (3/group) bearing subcutaneous SUM149 cell-derived xenografts were either untreated (No Rx, Control) or treated with Cisplatin (3 mg/kg/i.v. dose; day 1, 5, 9, 13), SNI-1 parent compound (70 mg/kg/i.p. dose, daily×13), SNI-1 Sodium salt (70 mg/kg/i.v. dose, daily×13), Cisplatin+SNI-1 (parent compound; matched schedules), and Cisplatin+SNI-1 (sodium salt; matched schedules). SNI-1 by either route (i.p. or i.v.) was well tolerated and both SNI-1 formulations and routes generated similar enhanced tumor growth suppression when combined with Cisplatin as compared with their respective single compound-treated arms (FIG. 24). These findings corroborate the results disclosed herein that demonstrate superior tumor growth inhibition by the combination of Cisplatin and SNI-1.

RENCA Cell-derived Syngeneic Kidney Cancer Tumor Model: In addition to the BRCA-mutant TNBC cancers, Cisplatin is also frequently used as a therapeutic for treatment of testicular, ovarian, cervical, bladder, head and neck, esophageal, and lung cancers, mesothelioma, brain tumors and neuroblastoma. In kidney cancers, genotoxic therapies such as Cisplatin, gemcitabine or 5-fluoracil are often used after treatments with targeted and/or immunotherapies. Since SNI-1 enhanced Cisplatin efficacy in TNBC tumors, it was investigated whether SNI-1 will also enhance efficacy of Cisplatin in renal cancer syngeneic tumor model. The data in FIG. 25 show that SNI-1+Cisplatin provoked a therapeutic response superior to single agent treatment in the RENCA renal cancer tumor model.

1. A method of treating cancer, the method comprising: administering to a subject with cancer a therapeutically effective amount of a CARP-1-NEMO inhibitor.

2. (canceled)

3. The method of claim 1, wherein the CARP-1-NEMO inhibitor is 1-(3,4-dihydroxyphenyl)-2-{(1-(4-methylphenyl)-1H-tetrazol-5-yl)thio}ethanone (SNI-1), a SNI-1 analog or 2-{((4-methoxyphenyl)sulfonyl)amino}-N-(2-phenylethyl)benzamide (SNI-2).

4.-6. (canceled)

7. The method of claim 1, wherein the cancer is brain cancer, breast cancer, renal cancer, pancreatic cancer, lung cancer, liver cancer, lymphoma, prostate cancer, colon cancer, ovarian cancer, or cervical cancer.

8.-13. (canceled)

14. The method of claim 1, wherein the CARP-1-NEMO inhibitor inhibits the binding of CARP-1 to NEMO.

15. The method of claim 1, wherein the CARP-1-NEMO inhibitor decreases or suppresses one or more pro-inflammatory cytokines.

16. (canceled)

17. The method of claim 15, wherein the decrease or suppression of the one or more pro-inflammatory cytokines reduces NF-κB activity.

18. The method of claim 1, further comprising administering a therapeutically effective amount of a chemotherapeutic agent or a DNA damage-inducing agent to the subject.

19. (canceled)

20. The method of claim 18, wherein administration of the CARP-1-NEMO inhibitor increases the efficacy of the chemotherapeutic agent or the DNA damage-inducing agent.

21.-23. (canceled)

24. A method of enhancing a chemotherapeutic response or reducing chemotherapeutic toxicity in a subject, the method comprising administering to a subject with cancer a therapeutically effective amount of a CARP-1-NEMO inhibitor and a therapeutically effective amount of chemotherapeutic agent or a DNA damage-inducing agent.

25.-44. (canceled)

45. A composition for treating cancer, the composition comprising a CARP-1-NEMO inhibitor, a DNA damage-inducing agent or a chemotherapeutic agent, and optionally, a pharmaceutical carrier; wherein the CARP-1-NEMO inhibitor and the DNA damaging chemotherapeutic agent are present in a therapeutically effective amount.

46.-48. (canceled)

49. The composition of claim 45, wherein the CARP-1-NEMO inhibitor is 1-(3,4-dihydroxyphenyl)-2-{(1-(4-methylphenyl)-1H-tetrazol-5-yl)thio}ethanone (SNI-1), SNI-1 or 2-{((4-methoxyphenyl)sulfonyl)amino}-N-(2-phenylethyl)benzamide (SNI-2).

50. The composition of claim 45, wherein the DNA damaging chemotherapeutic agent is doxorubicin, cisplatin, 5-flyoroyracil or etoposide.

51. (canceled)

52. A method of enhancing the efficacy of radiotherapy and/or a chemotherapeutic agent, the method comprising: administering to a subject with cancer: (a) an effective amount of radiotherapy, the chemotherapeutic agent, DNA damage-inducing agent or a combination thereof, and (b) a therapeutically effective amount of a CARP-1-NEMO inhibitor, wherein the administration of the CARP-1-NEMO inhibitor enhances the efficacy of the radiotherapy, the chemotherapeutic agent, DNA damage-inducing agent or a combination thereof in the subject with cancer.

53.-58. (canceled)

59. A method for screening one or more compounds for pharmacological intervention in cancer, the method comprising:
(a) providing a CARP-1 amino acid fragment capable of binding to a NEMO amino acid fragment or a NEMO amino acid fragment capable of binding to a CARP-1 amino acid fragment;
(b) providing a purified or non-purified compound or purified or non-purified mixture of compounds;
(c) screening the purified or non-purified compound or purified or non-purified mixture of compounds in an environment that allows for binding of the compound or mixture of compounds in (b) to the CARP-1 amino acid fragment or to the NEMO amino acid fragment; and
(d) isolating the compound or mixture of compounds in (c) that is bound to either the CARP-1 amino acid fragment or the NEMO amino acid fragment.

60.-62. (canceled)

63. The method of claim 59, wherein the CARP-1 amino acid fragment is
(SEQ ID NO: 6)
HRPEETHKGRTVPAHVETVVLFFPDVWHCL
or
(SEQ ID NO: 8)
AEIRYHRPEETHKGRTVPAHVETVVLFFPDVWHCL

64. The method of claim 59, wherein the NEMO amino acid fragment is
(SEQ ID NO: 7)
EEKRKLAQLQVAYHQLFQEYDNHIKSSVVGSERKRGMQLE.

65. (canceled)

66. (canceled)

67. The method of claim 1, wherein the CARP-1 NEMO inhibitor has a structure represented by a formula: 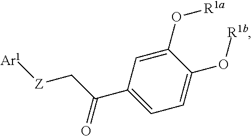
wherein Z is selected from —S—, —S(O)—, and —SO₂—;
wherein each of R^{1a} and R^{1b} is independently selected from hydrogen and C1-C4 alkyl,
or wherein each of R^{1a} and R^{1b} are covalently bonded, and, together with the intermediate atoms, comprise a 6-membered heterocycle;
or wherein each of R^{1a} and R^{1b} together comprise —CH₂—; and
wherein Ar is a 5- to 10-membered heteroaryl substituted with 0, 1, 2, or 3 substituents independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar²; and
wherein Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
provided that when Ar¹ is thiazole, then Ar¹ is substituted by at least 1 group,
provided that when Ar is thiazole and Z is —S—, then each of R^{1a} and R^{1b} is hydrogen,
provided that when Ar is benzo[d]thiazole and Z is —S—, then each of R^{1a} and R^{1b} is hydrogen, and
provided that when Ar is tetrazole, then Ar¹ has a structure represented by a formula: 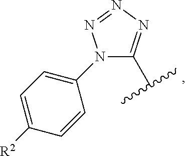
wherein R² is C1-C4 alkyl, and
Z is —S(O)— or —SO₂—,
or a pharmaceutically acceptable salt thereof.

68.-81. (canceled)

82. The method of claim 67, wherein the compound is selected from:

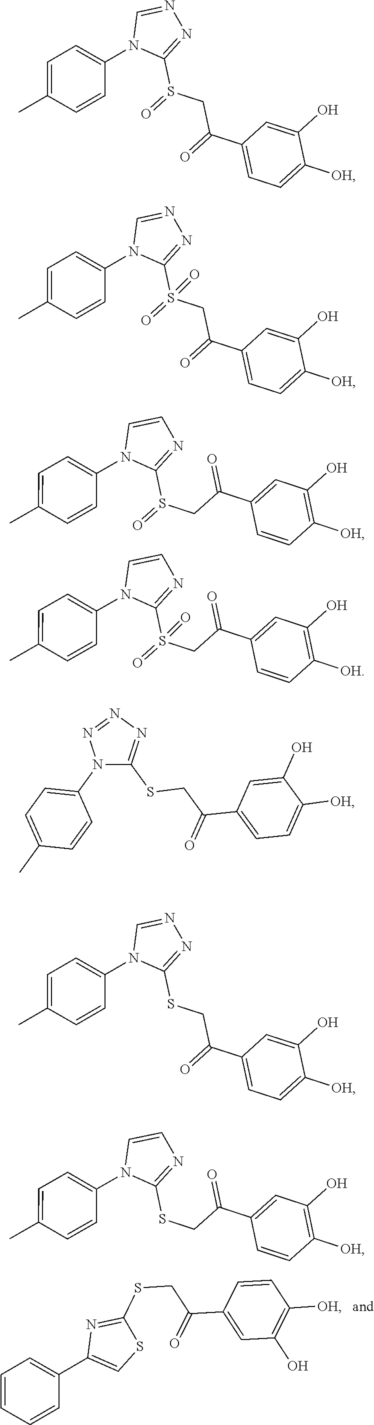

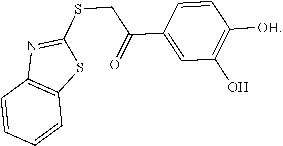 [SMILES: O=C(CSc1nc2ccccc2s1)c1ccc(O)c(O)c1]

83. A compound having a structure represented by a formula: 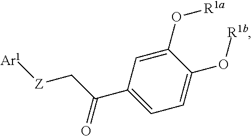
wherein Z is selected from —S(O)— and —SO₂—;
wherein each of R^{1a} and R is independently selected from hydrogen and C1-C4 alkyl,
or wherein each of R^{1a} and R^{1b} are covalently bonded, and, together with the intermediate atoms, comprise a 6-membered heterocycle;
or wherein each of R^{1a} and R^{1b} together comprise —CH₂—; and
wherein Ar¹ is a structure having a formula selected from: 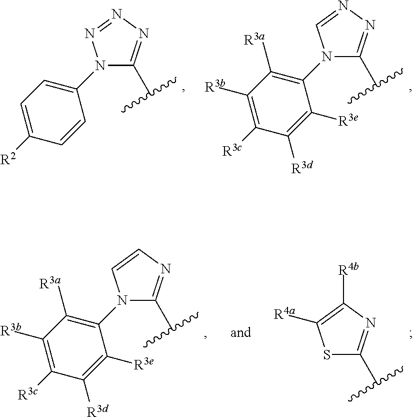
wherein R², when present, is C1-C4 alkyl;
wherein each of R^{3a}, R^{3b}, R^{3e}, R^{3d}, and R^{3e}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein each of R^{4a} and R^{4b}, when present, is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, and Ar², provided that at least one of R^{4a} and R^{4b}, when present, is not hydrogen; and
wherein Ar², when present, is selected from C6 aryl and C3-C5 heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
or wherein each of R^{4a} and R^{4b}, when present, are covalently bonded and, together with the intermediate atoms, comprise a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
or a pharmaceutically acceptable salt thereof.

84.-97. (canceled)

98. The compound of claim 83, wherein the compound is selected from:

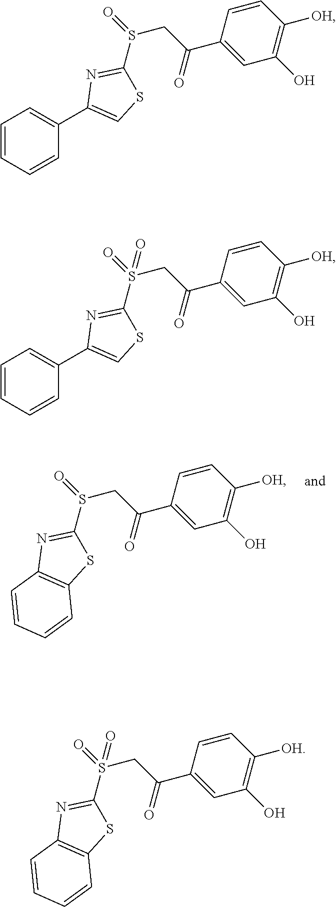 [SMILES: O=C(CS(=O)c1nc(-c2ccccc2)cs1)c1ccc(O)c(O)c1 ; O=C(CS(=O)(=O)c1nc(-c2ccccc2)cs1)c1ccc(O)c(O)c1 ; O=C(CS(=O)c1nc2ccccc2s1)c1ccc(O)c(O)c1 ; O=C(CS(=O)(=O)c1nc2ccccc2s1)c1ccc(O)c(O)c1]

99. The compound of claim 83, wherein the compound is selected from:

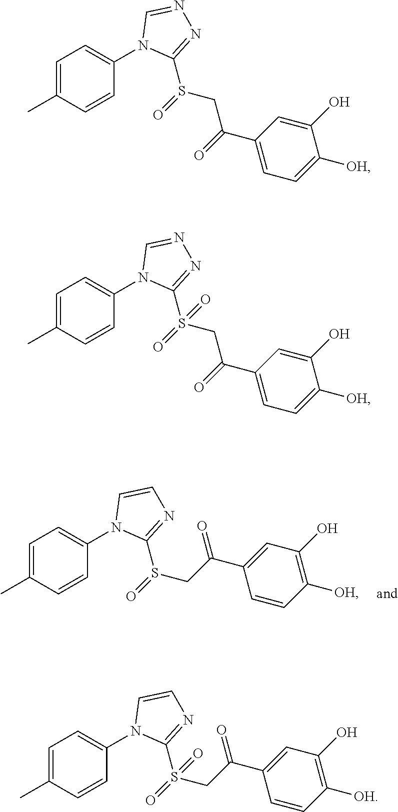 [SMILES: Cc1ccc(-n2cnnc2S(=O)CC(=O)c2ccc(O)c(O)c2)cc1 ; Cc1ccc(-n2cnnc2S(=O)(=O)CC(=O)c2ccc(O)c(O)c2)cc1 ; Cc1ccc(-n2ccnc2S(=O)CC(=O)c2ccc(O)c(O)c2)cc1 ; Cc1ccc(-n2ccnc2S(=O)(=O)CC(=O)c2ccc(O)c(O)c2)cc1]

100. (canceled)

101. (canceled)

102. A compound having a structure selected from: 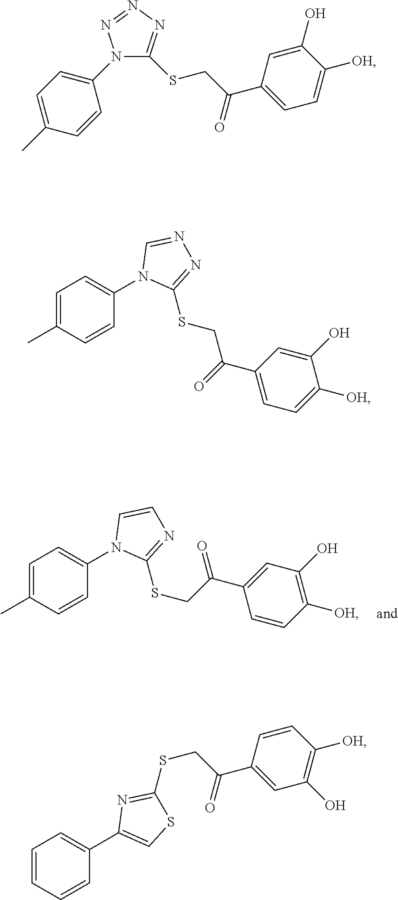 [SMILES: Cc1ccc(-n2nnnc2SCC(=O)c2ccc(O)c(O)c2)cc1 ; Cc1ccc(-n2cnnc2SCC(=O)c2ccc(O)c(O)c2)cc1 ; Cc1ccc(-n2ccnc2SCC(=O)c2ccc(O)c(O)c2)cc1 ; O=C(CSc1nc(-c2ccccc2)cs1)c1ccc(O)c(O)c1]
or a pharmaceutically acceptable salt thereof.

103. (canceled)

104. (canceled)
